(19) United States
(12) Patent Application Publication
(10) Pub. No.: US 2016/0221964 A1
(43) Pub. Date: Aug. 4, 2016

(54) FUNGICIDAL PYRIMIDINE COMPOUNDS

(71) Applicant: BASF SE, Ludwigshafen (DE)

(72) Inventors: Wassilios GRAMMENOS, Ludwigshafen (DE); Ian Robert CRAIG, Ludwigshafen (DE); Nadege BOUDET, Hirschberg (DE); Bernd MUELLER, Frankenthal (DE); Erica May Wilson LAUTERWASSER, Wachenheim (DE); Jan Klaas LOHMANN, Lambsheim (DE); Jurith MONTAG, Mannheim (DE); Thomas GROTE, Wachenheim (DE); Egon HADEN, Speyer (DE); Maria Angelica QUINTERO PALOMAR, Mannheim (DE); Ana ESCRIBANO CUESTA, Mannheim (DE)

(21) Appl. No.: 15/021,145

(22) PCT Filed: Sep. 16, 2013
(86) PCT No.: PCT/EP2013/069166

§ 371 Date: Mar. 10, 2016

(51) Int. Cl. C07D 239/42; A01N 43/54; C07D 471/04; A01N 43/90; C07D 513/04; C07D 401/12; C07D 487/04

(52) U.S. Cl. CPC C07D 239/42; C07D 401/12; A01N 43/54; C07D 487/04; A01N 43/90; C07D 513/04; C07D 471/04

(57) ABSTRACT

The present invention relates to fungicidal pyrimidine compounds I, to their use and to methods for combating phytopathogenic fungi. The present invention also relates to mixtures and compositions comprising compounds I and to seed treated with at least one compound I.

Furthermore the invention relates to processes for preparing compounds of formula I.

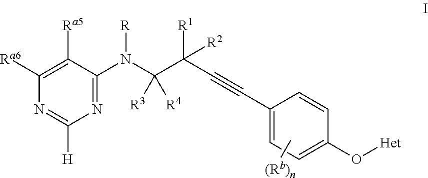

The present invention relates to fungicidal pyrimidine compounds I, to their use and to methods for combating phytopathogenic fungi. The present invention also relates to mixtures and compositions comprising compounds I and to seed treated with at least one compound I. Furthermore the invention relates to processes for preparing compounds of formula I.

WO 2011007839 A1 describes 4-(3-butynyl)aminopyrimidine derivatives, which are pest controlling agents for agricultural and horticultural use.

EP 264217 A2 discloses certain aralkylaminopyrimidine derivatives, which are useful as insecticides, acaricides and fungicides.

Prior right PCT/EP2013/054966 relates to fungicidal aminopyrimidine derivatives with a substituted alkynyl linker between the aminopyrimidine and the phenyl ring and discloses the compound 6-chloro-5-methoxy-N-[1-methyl-4-[4-[[4-(trifluoromethyl)-2-pyridyl]oxy]phenyl]but-3-ynyl]pyrimidin-4-amine.

The compounds according to the present invention differ from those described in the abovementioned publications in that the central phenyl ring is always substituted by a heteroaryloxy substituent situated in para-position with regard to the alkyne moiety, the pyrimidine ring is unsubstituted in position 2 and in that the linker between said phenyl ring and the aminopyrimidine moiety is a butynyl derived group as specifically described herein.

In many cases, in particular at low application rates, the fungicidal activity of known fungicidal compounds is unsatisfactory. Based on this, it was an object of the present invention to provide compounds having improved activity and/or a broader activity spectrum against phytopathogenic fungi. This objective is achieved by the use of substituted pyrimidine compounds of formula I having good fungicidal activity against phytopathogenic harmful fungi.

Accordingly, the present invention relates to compounds of the formula I

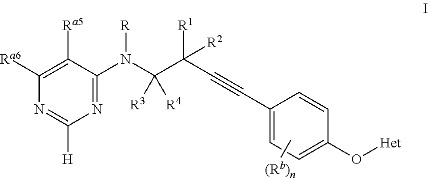

wherein:

R^{a5}, R^{a6} independently of each other are hydrogen, halogen, CN, NO₂, OH, SH, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₁-C₄-alkoxy-C₁-C₄-alkyl, C₁-C₄-alkoxy-C₁-C₄-alkoxy, C₂-C₄-alkenyl, C₂-C₄-alkynyl, C₂-C₄-haloalkenyl, C₂-C₄-haloalkynyl, C₃-C₈-cycloalkyl, C₃-C₈-cycloalkyloxy, C₃-C₈-cycloalkyl-C₁-C₄-alkyl, NR^{A}R^{B}, C(═O)R′, C(═NOR″)R′″ or —C(═NH)—O—R′″;
R^{A}, R^{B} independently of one another are hydrogen, C₁-C₄-alkyl, C₂-C₄-alkenyl, C₂-C₄-alkynyl, phenyl, benzyl, C₃-C₈-cycloalkyl, C₃-C₈-cycloalkenyl or —(C═O)—R′;
R′ is hydrogen, OH, NH₂, C₁-C₄-alkyl, C₁-C₄-haloalkyl, C₂-C₄-alkenyl, C₂-C₄-alkynyl, C₁-C₄-alkoxy, C₁-C₄-haloalkoxy, C₁-C₄-alkylamino or di(C₁-C₄-alkyl)amino;
R″ is hydrogen, C₁-C₄-alkyl, C₁-C₄-haloalkyl, C₂-C₄-alkenyl, C₂-C₄-alkynyl or C₁-C₄-alkoxy-C₁-C₄-alkyl;
R′″ is hydrogen or C₁-C₄-alkyl; or
R^{a5}, R^{a6} together with two ring member carbon atoms to which they are attached, form a fused 5-, 6- or 7-membered saturated, partially unsaturated or aromatic carbocycle or heterocycle, wherein the ring member atoms of the fused heterocycle include besides carbon atoms 1, 2, 3 or 4 heteroatoms selected from N, O and S, and wherein the fused carbocycle or heterocycle is unsubstituted or carries 1, 2, 3 or 4 identical or different radicals selected from halogen, CN, C₁-C₄-alkyl, C₁-C₄-alkoxy, C₁-C₄-haloalkyl and C₁-C₄-haloalkoxy;
R is hydrogen, C₁-C₄-alkyl, C₁-C₄-haloalkyl, C₁-C₄-alkoxy, C₁-C₄-haloalkoxy, C₁-C₄-alkoxy-C₁-C₄-alkyl, C₁-C₄-haloalkoxy-C₁-C₄-alkyl, C₂-C₄-alkenyl, C₂-C₄-haloalkenyl, C₂-C₄-alkynyl, C₂-C₄-haloalkynyl, CN, CH₂CN, NR^{A}R^{B} or CH₂—O—C(═O)R′;
R¹, R² independently of each other are hydrogen, halogen, CN, OH, C₁-C₄-alkyl, C₁-C₄-haloalkyl, C₁-C₄-alkoxy, C₁-C₄-haloalkoxy, C₁-C₄-alkoxy-C₁-C₄-alkyl, C₁-C₄-haloalkoxy-C₁-C₄-alkyl, C₂-C₄-alkenyl, C₂-C₄-haloalkenyl, C₂-C₄-alkynyl, C₂-C₄-haloalkynyl, C₃-C₈-cycloalkyl, C₃-C₈-cycloalkyl-C₁-C₄-alkyl, C₃-C₈-cycloalkyloxy, NR^{A}R^{B}, C(═O)R′, C(═NOR″)R′″, —C(═NH)—O—R′″ or benzyl wherein the phenyl moiety of benzyl is unsubstituted or carries 1, 2, 3, 4, or 5 substituents selected from CN, halogen, C₁-C₄-alkyl, C₁-C₄-haloalkyl, C₁-C₄-alkoxy, C₁-C₄-haloalkoxy, (C₁-C₄-alkoxy)carbonyl and di(C₁-C₄-alkyl)aminocarbonyl; or
   two radicals R¹ and R² that are bound to the same carbon atom form together with said carbon atom a saturated or partially unsaturated 3-, 4-, 5-, 6-, or 7-membered carbocycle or a saturated or partially unsaturated 3-, 4-, 5-, 6-, or 7-membered heterocycle, wherein the ring member atoms of the abovementioned heterocycle include beside carbon atoms 1, 2, 3 or 4 heteroatoms selected from N, O and S, and wherein the abovementioned cycle is unsubstituted or carries 1, 2, 3 or 4 substituents selected from halogen, CN, OH, SH, C₁-C₄-alkyl, C₁-C₄-alkoxy and C₁-C₄-alkylthio; and one or two CH₂ groups of the abovementioned cycles may respectively be replaced by one or two C(═O) or C(═S) groups;
R³, R⁴ independently of each other are hydrogen, CN, C₁-C₄-hydroxyalkyl, C₁-C₄-alkyl, C₁-C₄-haloalkyl, C₁-C₄-alkoxy, C₁-C₄-haloalkoxy, C₁-C₄-alkoxy-C₁-C₄-alkyl, C₁-C₄-halo-alkoxy-C₁-C₄-alkyl, C₂-C₄-alkenyl, C₂-C₄-haloalkenyl, C₂-C₄-alkynyl, C₂-C₄-haloalkynyl, C₃-C₈-cycloalkyl, C₃-C₈-cycloalkyl-C₁-C₄-alkyl, C₃-C₈-cycloalkyloxy, NR^{A}R^{B}, C(═O)R′, C(═NOR″)R′″, —C(═NH)—O—R′″ or benzyl wherein the phenyl moiety of benzyl is unsubstituted or carries 1, 2, 3, 4, or 5 substituents selected from the group consisting of CN, halogen, C₁-C₄-alkyl, C₁-C₄-haloalkyl, C₁-C₄-alkoxy, C₁-C₄-haloalkoxy, (C₁-C₄-alkoxy)carbonyl and di(C₁-C₄-alkyl)aminocarbonyl; or two radicals R³ and R⁴ that are bound to the same carbon atom form together with said carbon atom a saturated or partially unsaturated 3-, 4-, 5-, 6-, or 7-membered carbocycle or a saturated or partially unsaturated 3-, 4-, 5-,6-, or 7-membered heterocycle, wherein the ring member atoms of the abovementioned heterocycle include beside carbon atoms 1, 2, 3 or 4 heteroatoms selected from the group of N, O and S, and wherein the abovementioned cycle is unsubstituted or carries 1, 2, 3 or 4 substituents selected from halogen, CN, OH, SH, C₁-C₄-alkyl, C₁-C₄-alkoxy or C₁-C₄-alkylthio; and one or two CH₂ groups of the abovementioned cycles may be respectively replaced by one or two C(═O) or C(═S) groups;
n is 0, 1, 2, 3 or 4;
R^{b} is independently selected from halogen, CN, NO₂, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl, C₂-C₄-haloalkynyl, NR^{A}R^{B}, C(═O)R′, C(═NOR″)R′″ and —C(═NH)—O—R′″;
Het is a 5- or 6-membered heteroaryl, wherein the ring member atoms of the heteroaryl include besides carbon atoms 1, 2, 3 or 4 heteroatoms selected from N, O and S, and wherein the heteroaryl is unsubstituted or carries 1, 2, 3 or 4 identical or different groups R^{c}:
   R^{c} is halogen, CN, NO₂, NH₂, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C(═O)R′, C(═NOR″)R′″, C₃-C₈-cycloalkyl, C₃-C₈-cycloalkyl-C₁-C₄-alkyl, phenyl, phenoxy, phenoxy-C₁-C₄-alkyl or a 5- or 6-membered heteroaryl, wherein the ring member atoms of the heteroaryl include besides carbon atoms 1, 2, 3 or 4 heteroatoms selected from N, O and S, and wherein the aforementioned cyclic radicals are unsubstituted or carry 1, 2, 3 or 4 identical or different substituents R^{d}:
      R^{d} is halogen, CN, C₁-C₄-alkyl, C₁-C₄-haloalkyl, C₁-C₄-alkoxy or C₁-C₄-haloalkoxy;
   or two radicals R^{c} that are bound to adjacent ring member atoms of the Het group form together with said ring member atoms a fused
   5-, 6- or 7-membered saturated, partially unsaturated or aromatic carbocycle or heterocycle, wherein the ring member atoms of the fused heterocycle include besides carbon atoms 1, 2, 3 or 4 heteroatoms selected from the group of N, O and S, and wherein the fused carbocycle or heterocycle is unsubstituted or carries 1, 2, 3 or 4 identical or different radicals groups R^{e}:
   R^{e} is halogen, CN, C₁-C₄-alkyl, C₁-C₄-haloalkyl, C₁-C₄-alkoxy or C₁-C₄-haloalkoxy;

and the N-oxides and the agriculturally acceptable salts of the compounds of formula I, with the proviso that 6-chloro-5-methoxy-N-[1-methyl-4-[4-[[4-(trifluoromethyl)-2-pyridyl]oxy]phenyl]but-3-ynyl]pyrimidin-4-amine be excluded.

The compounds of the present invention are useful for combating harmful fungi. Therefore the present invention furthermore relates to a method for combating harmful fungi, which process comprises treating the fungi or the materials, plants, the soil or seeds to be protected against fungal attack, with an effective amount of at least one compound of formula I or of an N-oxide or an agriculturally acceptable salt thereof.

Furthermore, the present invention relates to plant propagation material, coated with a compound of formula I, or an N-oxide or an agriculturally acceptable salt thereof, or mixtures and compositions comprising compounds I. Furthermore, the present invention also relates to seed comprising or treated with a compound of formula I, or an N-oxide or an agriculturally acceptable salt thereof, or an agrochemical composition comprising a compound I, or an N-oxide or an agriculturally acceptable salt thereof, and seed in an amount of from 0.1 g to 10 kg per 100 kg of seed.

The present invention furthermore relates to processes for preparing compounds of formula I. The present invention furthermore relates to intermediates such as compounds of formulae III and to processes for preparing them.

Accordingly a 4-halopyrimidine compound II, wherein Hal is halogen, preferably Cl or F, can be reacted with a suitable amine compound III to obtain a compound I according to the present invention as shown in scheme 1.

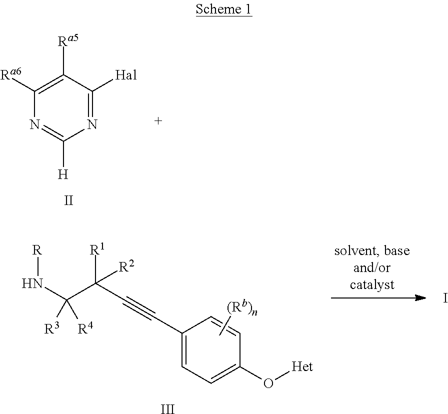

Generally, this reaction is carried out at temperatures of from 0 to 200° C., preferably from 50 to 170° C., preferably in an inert organic solvent and preferably in presence of a base or a catalyst or a combination of a base and a catalyst.

Suitable catalysts are e.g. metal halides such as NaF, KF, LiF, NaBr, KBr, LiBr, NaI, KI, LiI; ionic liquids, such as imidazolium catalysts; transition metal catalysts like palladium, rhodium, ruthenium, iron, copper in the form of halides, pseudohalides, alkoxides, carboxylates (preferred acetate), complexes with dibenzylidene acetone and ligands like phosphine, phosphites, phosphoramidate ligands. Preferred ligands are bidentate and sterically demanding phosphorous ligands, even more preferably the catalysts are selected from 2,2′ bis(diphenylphosphanyl)-1,1′-binaphthyl, 2,2′-Bis(diphenylphosphino)-1,1′-biphenyl, 2,4′,6′-diisopropyl-1,1′-biphenyl-2-yldicyclohexylphosphine, 2-(dicyclohexylphosphino)-2′,6′ dimethoxy-1,1′ bi-phenyl, 1,1-bis(diphenylphosphino)ferrocene, 9,9-dimethyl-4,5 bis(diphenylphosphino)xanthene, 1,2-bis(diphenylphosphino)ethane (dppe), 1,3-propanediylbis[diphenylphosphine], 1,4 butanediylbis[diphenylphosphine] and 1,1′-(1,2-ethanediyl)bis[1-(2-methoxyphenyl)-1 phenyl-diposphine.

Suitable solvents are aromatic hydrocarbons such as toluene, o-, m- and p-xylene; halogenated hydro-carbons chlorobenzene, dichlorobenzene; ethers such as dioxane, anisole and THF; nitriles such as acetonitrile and propionitrile; ketones such as acetone, methyl ethyl ketone, diethyl ketone and tert.-butyl methyl ketone; alcohols such as ethanol, n-propanol, isopropanol, n-butanol and tert.-butanol; and also DMSO, DMF, dimethyl acetamide, NMP, NEP and acetic acid ethyl ester, preferably THF, DMSO, DMF, dimethyl acetamide, NMP or NEP; even more preferably THF, DMF or NMP. It is also possible to use mixtures of the solvents mentioned.

Suitable bases are, in general, inorganic compounds, such as alkali metal and alkaline earth metal hydroxides such as lithium hydroxide, sodium hydroxide, potassium hydroxide and calcium hydroxide; alkali metal and alkaline earth metal oxides such as lithium oxide, sodium oxide, potassium oxide and calcium oxide; alkali metal and alkaline earth metal phosphates such as lithium phosphate, sodium phosphate, potassium phosphate and calcium phosphate; alkali metal amides such as lithium amide, sodium amide and potassium amide; alkali metal and alkaline earth metal hydrides lithium hydride, sodium hydride, potassium hydride and calcium hydride; alkali metal and alkaline earth metal carbonates such as lithium carbonate, potassium carbonate and calcium carbonate, caesium carbonate; moreover organic bases, for example tertiary amines such as trimethyl-amine (TMA), triethylamine (TEA), tributylamine (TBA), diisopropylethylamine (DIPEA) and N-methyl-2-pyrrolidone (NMP), pyridine, substituted pyridines such as collidine, lutidine and 4 dimethylaminopyridine (DMAP), and also bicyclic amines. Preference is given to sodium hydride, potassium hydride, lithium carbonate, potassium carbonate, caesium carbonate, TEA, TBA and DIPEA, in particular DIPEA. The bases are generally employed in equimolar amounts, in excess or, if appropriate, as solvent. The amount of base is typically 1.1 to 5.0 molar equivalents relative to 1 mole of compounds II.

The starting materials are generally reacted with one another in equimolar amounts. In terms of yields, it may be advantageous to employ an excess of compounds III, based on 1.1 to 2.5 equivalents, preferred 1.1 to 1.5 equivalents of compounds II.

The compounds II are known from the literature or are commercially available or they can be prepared for example in analogy to methods described in: Heterocycles (2009) 78(7), 1627-1665; New J. Chem. (1994) 18(6), 701-8; WO 2005/095357; Science of Synthesis (2004) 16, 379-572; WO 2008/156726; WO 2006/072831; Organic Reactions (Hoboken, N.J., United States) (2000), 56; or Targets in Heterocyclic Systems (2008) 12, 59-84.

The alkyne amine compounds III are known from the literature or are commercially available or they can be prepared for example in analogy to methods described in WO 2011007839.

The compounds III can also be prepared for example in analogy to methods described in scheme 2, wherein PG in compound AD-2 stands for a suitable protection group for an amine, for example tert-butoxycarbonyl, benzyloxy carbonyl, benzyl, 4-methoxy benzyl, acetyl or trichloro acetyl. According to scheme 2, butyne compounds can be synthesized via a palladium catalyzed crosscoupling of an aryl halide AD-1 with suitable alkynes AD-2 (US 20110105562 A1, Tetrahedron (1992), 48(15), 3239-50; WO 2004043458 A1); the heterocycle Het can be installed before or after the crosscoupling reaction.

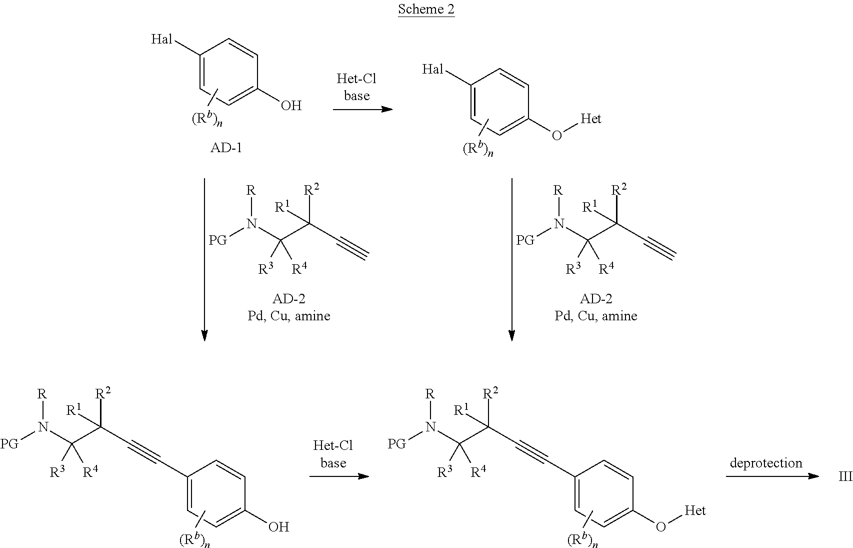

Alkynes AD-2 are commercially available or they can be synthesized according to scheme 3.

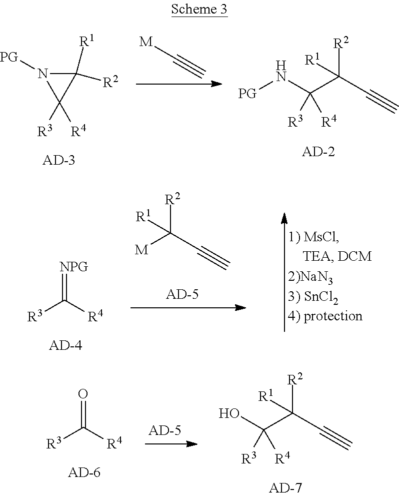

Ring opening of a substituted aziridine AD-3 with a metal acetylide, wherein M can be, for example, lithium, directly leads to the formation of AD-2 (Angewandte Chemie, International Edition (2011), 50(9), 2144-2147; Journal of the American Chemical Society (2010), 132(13), 4542-4543; Organic Letters (2007), 9(24), 5127-5130; WO 2006044412 A1). Nucleophilic addition of a propargyl metal AD-5, wherein M can be, for example, lithium, to an imine AD-4 is another way to prepare the amine compound AD-2 (European Journal of Organic Chemistry (2010), (8), 1587-1592; Synlett (2008), (4), 578-582; Journal of Organic Chemistry (1999), 64(7), 2406-2410; Synthetic Communications (1997), 27(15), 2601-2614).

Alkohols AD-7 can be used to synthesize amines AD-2. Alcohols AD-7 are commercially available or methods for their preparation are described in the literature.

Conversion of AD-7 to an amine can be achieved in a three step reaction sequence comprising a) mesylation with methyanesulfonic acid chloride (MsCI)) in the presence of a base such as triethylamine, b) treatment of the intermediate methylsulfonate with sodium azide, and c), subsequent reduction of the alkylazide with a suitable reductant (e.g. SnCl₂; as described in Journal of Medicinal Chemistry (2011), 54(20), 7363-7374; WO 2011098603 A1, Bioorganic & Medicinal Chemistry (2011), 19(10), 3274-3279) followed by protection of the amnio group. It is also possible to synthesize such compounds under Mitsunobu conditions as described in Journal of Organic Chemistry (2011), 76(14), 5661-5669 or Chemistry-A European Journal (2011), 17(6), 1764-1767 or by way of a Gabriel synthesis as described in European Journal of Medicinal Chemistry (2011), 46(8), 3227-3236, Chemistry-A European Journal (2010), 16(41), 12303-12306 or in WO 2010017047 A1.

Compounds II, wherein R^{a5} and R^{a6} in each case constitute together with two ring member carbon atoms of the pyrimidine ring one of the following heterocyclic groups as defined in line 1 to line 26 in table A.1, wherein #5 and #6 indicate the point of attachment to the pyrimidine ring, each respectively corresponding to the positions of either substituent R^{a5} or R^{a6}, can be prepared according to commonly known procedures such as those given below or in analogy to those cited references or are commercially available.

TABLE A.1

| line | R^{a5}/R^{a6} |
|---|---|
| A.1-1 | #5—CH═CH—CH═CH—#6 |
| A.1-2 | #5—CH₂—CH₂—CH₂—CH₂—#6 |
| A.1-3 | #5—CH═CH—CH═N—#6 |
| A.1-4 | #5—N═CH—CH═CH—#6 |
| A.1-5 | #5—CH═N—CH═N—#6 |
| A.1-6 | #5—N═CH—N═CH—#6 |
| A.1-7 | #5—CH₂—CH₂—CH₂—#6 |
| A.1-8 | #5—N═CH—CH═N—#6 |
| A.1-9 | #5—O—CH₂—O—#6 |
| A.1-10 | #5—NH—CH═N—#6 |
| A.1-11 | #5—S—CH═N—#6 |
| A.1-12 | #5—N═CH—S—#6 |
| A.1-13 | #5—O—CH═N—#6 |
| A.1-14 | #5—N═CH—O—#6 |
| A.1-15 | #5—O—CH═CH—#6 |
| A.1-16 | #5—S—CH═CH—#6 |
| A.1-17 | #5—O—N═CH—#6 |
| A.1-18 | #5—S—N═CH—#6 |
| A.1-19 | #5—CH═N—O—#6 |
| A.1-20 | #5—CH═N—S—#6 |
| A.1-21 | #5—N(CH₃)—CH═CH—#6 |
| A.1-22 | #5—CH═CH—N(CH₃)—#6 |
| A.1-23 | #5═CH—N(NH₂)—N═#6 |
| A.1-24 | #5—CH═N—N(CH₃)—#6 |
| A.1-25 | #5═N—N(CH₃)—CH═#6 |
| A.1-26 | #5—N(CH₃)—N═CH—#6 |

Compounds II wherein the meaning of R^{a5} and R^{a6} corresponds to line 1 of table A.2 can be prepared as described in EP 326329 A2, US 20050187231 A1, WO 2007071963 A2, Tetrahedron (2004), 60(25), 5373-5382, Bioorganic & Medicinal Chemistry Letters (2009), 19(6), 1715-1717 or in WO 2010025451 A2.

Compounds II wherein the meaning of R^{a5} and R^{a6} corresponds to line 3 of table A.2 can be prepared as described in European Journal of Medicinal Chemistry (2011), 46(9), 3887-3899, WO 2011104183 A1 or in Organic Process Research & Development (2011), 15(4), 918-924.

Compounds II wherein the meaning of R^{a5} and R^{a6} corresponds to line 4 of table A.2 can be prepared as described in WO 2011131741 A1, WO 2010101949 A1 or in Journal of Organic Chemistry (1979), 44(3), 435-40.

Compounds II wherein the meaning of R^{a5} and R^{a6} corresponds to line 5 or 20 of table A.2 can be prepared as described in Organic Process Research & Development (2011), 15(4), 918-924; or in Tetrahedron (1998), 54(33), 9903-9910.

Compounds II wherein the meaning of R^{a5} and R^{a6} corresponds to line 6 of table A.2 can be prepared as described in WO 2010026262 A1 or in WO 2007092681 A2.

Compounds II wherein the meaning of R^{a5} and R^{a6} corresponds to line 8 of table A.2 can be prepared as described in WO 2010038060 A1, Bioorganic & Medicinal Chemistry Letters (2010), 20(7), 2330-2334, CN 101544642 A or in Journal of the American Chemical Society (1956), 78, 225-8.

Compounds II wherein the meaning of R^{a5} and R^{a6} corresponds to line 11 of table A.2 can be prepared as described in US 20110028496 A1.

Compounds II wherein the meaning of R^{a5} and R^{a6} corresponds to line 12 of table A.2 can be prepared as described in WO 2010014930, US 20110028496 A1 or in WO 2008057402 A2.

Compounds II wherein the meaning of R^{a5} and R^{a6} corresponds to line 14 of table A.2 can be prepared as described in Australian Journal of Chemistry (1990), 43(1), 47-53 or in WO 2009013545 A2.

Compounds II wherein the meaning of R^{a5} and R^{a6} corresponds to line 24 of table A.2 can be prepared as described in US 20090005359 A1 or in US 20070281949 A1.

Compounds II wherein the meaning of R^{a5} and R^{a6} corresponds to line 25 of table A.2 can be prepared as described in WO 2007013964 A1.

Compounds II wherein the meaning of R^{a5} and R^{a6} corresponds to line 26 of table A.2 can be prepared as described in Journal of Medicinal Chemistry (1988), 31(2), 454-61 or in WO 2006046135 A2.

If individual compounds I cannot be obtained by the routes described above, they can be prepared by derivatization of other compounds I. The N-oxides may be prepared from the compounds I according to conventional oxidation methods, e. g. by treating compounds I with an organic peracid such as metachloroperbenzoic acid (cf. WO 03/64572 or J. Med. Chem. (1995), 38(11), 1892-1903,); or with inorganic oxidizing agents such as hydrogen peroxide (cf. J. Heterocyc. Chem. (1981), 18 (7), 1305-1308) or oxone (cf. J. Am. Chem. Soc. (2001), 123 (25), 5962-5973). The oxidation may lead to pure mono-N-oxides or to a mixture of different N-oxides, which can be separated by conventional methods such as chromatography.

If the synthesis yields mixtures of isomers, a separation is generally not necessarily required since in some cases the individual isomers can be interconverted during work-up for use or during application (e. g. under the action of light, acids or bases). Such conversions may also take place after use, e. g. in the treatment of plants in the treated plant, or in the harmful fungus to be controlled.

The reaction mixtures are worked up in a customary manner, for example by mixing with water, separating the phases and, if appropriate, chromatic purification of the crude products. In some cases, the intermediates and end products are obtained in the form of colorless or slightly viscous oils which can be freed from volatile components or purified under reduced pressure and at moderately elevated temperatures. If the intermediates and end products are obtained as solids, purification can also be carried out by recrystallization or digestion. Depending on the substitution pattern, the compounds of formula I and their N-oxides may have one or more centers of chirality, in which case they are present as pure enantiomers or pure diastereomers or as enantiomer or diastereomer mixtures. Both, the pure enantiomers or diastereomers and their mixtures are subject matter of the present invention.

Compounds I can be present in different crystal modifications whose biological activity may differ. They also form part of the subject matter of the present invention. The compounds of formula I can be present in atropisomers arising from restricted rotation about a single bond of asymmetric groups. They also form part of the subject matter of the present invention.

Agriculturally useful salts of the compounds I encompass especially the salts of those cations or the acid addition salts of those acids whose cations and anions, respectively, have no adverse effect on the fungicidal action of the compounds I. Suitable cations are thus in particular the ions of the alkali metals, preferably sodium and potassium, of the alkaline earth metals, preferably calcium, magnesium and barium, of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting a compound I with an acid of the corresponding anion, preferably of hydrochloric acid, hydrobromic acid, sulfuric acid, phosphoric acid or nitric acid.

In respect of the variables, the embodiments of the intermediates correspond to the embodiments of the compounds of formula I. The term “compounds I” refers to compounds of formula I. Likewise, the term “compounds II” refers to compounds of formula II.

In the definitions of the variables given above, collective terms are used which are generally representative for the substituents in question. The term “Cₙ-Cₘ” indicates the number of carbon atoms possible in each case in the substituent or substituent moiety in question.

The term “halogen” refers to fluorine, chlorine, bromine and iodine.

The term “C₁-C₄-alkyl” refers to a straight-chained or branched saturated hydrocarbon group having 1 to 4 carbon atoms, for example methyl, ethyl, propyl, 1-methylethyl, butyl, 1-methylpropyl, 2-methylpropyl, and 1,1-dimethylethyl. Likewise, the term “C₁-C₆-alkyl” refers to a straight-chained or branched saturated hydrocarbon group having 1 to 6 carbon atoms.

The term “C₁-C₄-haloalkyl” refers to a straight-chained or branched alkyl group having 1 to 4 carbon atoms (as defined above), wherein some or all of the hydrogen atoms in these groups may be replaced by halogen atoms as mentioned above, for example chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂—C₂F₅, CF₂—C₂F₅, CF(CF₃)₂, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl. Likewise, the term “C₁-C₆-haloalkyl” refers to a straight-chained or branched alkyl group having 1 to 6 carbon atoms.

The term “C₁-C₄-alkoxy” refers to a straight-chain or branched alkyl group having 1 to 4 carbon atoms (as defined above) which is bonded via an oxygen, at any position in the alkyl group, for example methoxy, ethoxy, n-propoxy, 1-methylethoxy, butoxy, 1-methyl [symbol] propoxy, 2-methylpropoxy or 1,1-dimethylethoxy. Likewise, the term “C₁-C₆-alkoxy” refers to a straight-chain or branched alkyl group having 1 to 6 carbon atoms.

The term “C₁-C₄-hydroxyalkyl” refers to a straight-chained or branched alkyl group having 2 to 4 carbon atoms (as defined above), wherein one hydrogen atom in these groups may be replaced by one hydroxy group, for example hydroxymethyl, 2-hydroxyethyl, 3-hydroxy-1-propyl, 4-hydroxy-butyl.

The term “C₁-C₄-haloalkoxy” refers to a C₁-C₄-alkoxy group as defined above, wherein some or all of the hydrogen atoms may be replaced by halogen atoms as mentioned above, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromo-ethoxy, 4-fluorobutoxy, 4-chlorobutoxy, 4-bromobutoxy or nonafluorobutoxy. Likewise, the term “C₁-C₆-haloalkoxy” refers to a C₁-C₆-alkoxy group as defined above, wherein some or all of the hydrogen atoms may be replaced by halogen atoms as mentioned above.

The term “C₁-C₄-alkoxy-C₁-C₄-alkyl” refers to alkyl having 1 to 4 carbon atoms (as defined above), wherein one hydrogen atom of the alkyl radical is replaced by a C₁-C₄-alkoxy group (as defined above). Likewise, the term “C₁-C₆-alkoxy-C₁-C₄-alkyl” refers to alkyl having 1 to 4 carbon atoms (as defined above), wherein one hydrogen atom of the alkyl radical is replaced by a C₁-C₆-alkoxy group (as defined above).

The term “C₁-C₄-haloalkoxy-C₁-C₄-alkyl” refers to alkyl having 1 to 4 carbon atoms (as defined above), wherein one hydrogen atom of the alkyl radical is replaced by a C₁-C₄-haloalkoxy group (as defined above). Likewise, the term “C₁-C₆-haloalkoxy-C₁-C₄-alkyl” refers to alkyl having 1 to 4 carbon atoms (as defined above), wherein one hydrogen atom of the alkyl radical is replaced by a C₁-C₆-alkoxy group (as defined above).

The term “C₁-C₄-alkylthio” as used herein refers to straight-chain or branched alkyl groups having 1 to 4 carbon atoms (as defined above) bonded via a sulfur atom, at any position in the alkyl group, for example methylthio, ethylthio, propylthio, isopropylthio, and n butylthio.

Likewise, the term “C₁-C₆-alkylthio” as used herein refers to straight-chain or branched alkyl groups having 1 to 6 carbon atoms (as defined above) bonded via a sulfur atom. Accordingly, the terms “C₁-C₄-haloalkylthio” and “C₁-C₆-haloalkylthio” as used herein refer to straight-chain or branched haloalkyl groups having 1 to 4 or 1 to 6 carbon atoms (as defined above) bonded through a sulfur atom, at any position in the haloalkyl group.

The terms “C₁-C₄-alkylsulfinyl” or “C₁-C₆-alkylsulfinyl” refer to straight-chain or branched alkyl groups having 1 to 4 or 1 to 6 carbon atoms (as defined above) bonded through a —S(═O)— moiety, at any position in the alkyl group, for example methylsulfinyl and ethylsulfinyl, and the like. Accordingly, the terms “C₁-C₄-haloalkylsulfinyl” and “C₁-C₆-haloalkylsulfinyl”, respectively, refer to straight-chain or branched haloalkyl groups having 1 to 4 and 1 to 6 carbon atoms (as defined above), respectively, bonded through a —S(═O)— moiety, at any position in the haloalkyl group.

The terms “C₁-C₄-alkylsulfonyl” and “C₁-C₆-alkylsulfonyl”, respectively, refer to straight-chain or branched alkyl groups having 1 to 4 and 1 to 6 carbon atoms (as defined above), respectively, bonded through a —S(═O)₂— moiety, at any position in the alkyl group, for example methylsulfonyl. Accordingly, the terms “C₁-C₄-haloalkylsulfonyl” and “C₁-C₆-haloalkylsulfonyl”, respectively, refer to straight-chain or branched haloalkyl groups having 1 to 4 and 1 to 6 carbon atoms (as defined above), respectively, bonded through a —S(═O)₂— moiety, at any position in the haloalkyl group.

The term “C₁-C₄-alkylamino” refers to an amino radical carrying one C₁-C₄-alkyl group (as defined above) as substituent, for example methylamino, ethylamino, propylamino, 1-methylethylamino, butylamino, 1-methylpropylamino, 2-methylpropylamino, 1,1-di-methylethylamino and the like. Likewise, the term “C₁-C₆-alkylamino” refers to an amino radical carrying one C₁-C₆-alkyl group (as defined above) as substituent.

The term “di(C₁-C₄-alkyl)amino” refers to an amino radical carrying two identical or different C₁-C₄-alkyl groups (as defined above) as substituents, for example dimethylamino, diethylamino, di-n-propylamino, diisopropylamino, N-ethyl-N-methylamino, N-(n-propyl)-N-methylamino, N-(isopropyl)-N methylamino, N-(n-butyl)-N-methylamino, N-(n-pentyl)-N-methylamino, N-(2-butyl)-N methylamino, N-(isobutyl)-N-methylamino, and the like. Likewise, the term “di(C₁-C₆-alkyl)amino” refers to an amino radical carrying two identical or different C₁-C₆-alkyl groups (as defined above) as substituents.

The term “(C₁-C₄-alkoxy)carbonyl” refers to a C₁-C₄-alkoxy radical (as defined above) which is attached via a carbonyl group.

The term “di(C₁-C₄-alkyl)aminocarbonyl” refers to a di(C₁-C₄)alkylamino radical as defined above which is attached via a carbonyl group.

The term “phenoxy” and refers to a phenyl radical which is attached via an oxygen atom.

Likewise, the term “phenoxy-C₁-C₄-alkyl” and refers to a phenoxy radical which is attached via a C₁-C₄-alkyl group (as defined above).

The term “C₂-C₄-alkenyl” refers to a straight-chain or branched unsaturated hydrocarbon radical having 2 to 4 carbon atoms and a double bond in any position, such as ethenyl, 1-propenyl, 2-propenyl (allyl), 1-methylethenyl, 1-butenyl, 2-butenyl, 3-butenyl, 1-methyl-1-propenyl, 2-methyl-1-propenyl, 1-methyl-2-propenyl, 2-methyl-2-propenyl. Likewise, the term “C₂-C₆-alkenyl” refers to a straight-chain or branched unsaturated hydrocarbon radical having 2 to 6 carbon atoms and a double bond in any position.

The term “C₂-C₄-alkynyl” refers to a straight-chain or branched unsaturated hydrocarbon radical having 2 to 4 carbon atoms and containing at least one triple bond, such as ethynyl, 1-propynyl, 2-propynyl, 1-butynyl, 2-butynyl, 3-butynyl, 1-methyl-2-propynyl. Likewise, the term “C₂-C₆-alkynyl” refers to a straight-chain or branched unsaturated hydrocarbon radical having 2 to 6 carbon atoms and at least one triple bond.

The term “C₃-C₈-cycloalkyl” refers to monocyclic saturated hydrocarbon radicals having 3 to 8 carbon ring members, such as cyclopropyl (C₃H₅), cyclobutyl, cyclopentyl, cyclohexyl, cycloheptyl or cyclooctyl.

The term “C₃-C₈-cycloalkyl-C₁-C₄-alkyl” refers to a cycloalkyl radical having 3 to 8 carbon atoms (as defined above), which is bonded via a C₁-C₄-alkyl group (as defined above).

The term “C₃-C₈-cycloalkyloxy” refers to a cycloalkyl radical having 3 to 8 carbon atoms (as defined above), which is bonded via an oxygen.

The term “saturated or partially unsaturated 3-, 4-5-, 6- or 7-membered carbocycle” is to be understood as meaning both saturated or partially unsaturated carbocycles having 3, 4, 5, 6 or 7 ring members. Examples include cyclopropyl, cyclopentyl, cyclopentenyl, cyclopentadienyl, cyclohexyl, cyclohexenyl, cyclohexadienyl, cycloheptyl, cycloheptenyl, cycloheptadienyl, and the like.

The term “saturated or partially unsaturated 3-, 4-, 5-, 6-, or 7-membered heterocycle, wherein the ring member atoms of the heterocycle include besides carbon atoms 1, 2, 3 or 4 heteroatoms selected from the group of N, O and S”, is to be understood as meaning both saturated and partially unsaturated heterocycles, for example:
a 3- or 4-membered saturated heterocycle which contains 1 or 2 heteroatoms from the group consisting of N, O and S as ring members such as oxirane, aziridine, thiirane, oxetane, azetidine, thiethane, [1,2]dioxetane, [1,2]dithietane, [1,2]diazetidine; and
a 5- or 6-membered saturated or partially unsaturated heterocycle which contains 1, 2 or 3 heteroatoms from the group consisting of N, O and S as ring members such as 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding -ylidene radicals; and

a 7-membered saturated or partially unsaturated heterocycle such as tetra- and hexahydroazepinyl, such as 2,3,4,5-tetrahydro[1H]azepin-1-,-2-,-3-,-4-,-5-,-6- or -7-yl, 3,4,5,6-tetrahydro[2H]azepin-2-,-3-,-4-,-5-,-6- or -7-yl, 2,3,4,7-tetrahydro[1H]azepin-1-, 2-,-3-,-4-,-5-,-6- or -7-yl, 2,3,6,7-tetrahydro[1H]azepin-1-,-2-,-3-,-4-,-5-,-6- or -7-yl, hexahydroazepin-1-,-2-,-3- or -4-yl, tetra- and hexahydrooxepinyl such as 2,3,4,5-tetrahydro[1H]oxepin-2-,-3-,-4-,-5-,-6- or -7-yl, 2,3,4,7-tetrahydro[1H]oxepin-2-,-3-,-4-,-5-,-6- or -7-yl, 2,3,6,7-tetrahydro[1H]oxepin-2-, -3-,-4-,-5-,-6- or -7-yl, hexahydroazepin-1-,-2-,-3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding -ylidene radicals; and

The term “5- or 6--membered heteroaryl, wherein the ring member atoms of the heteroaryl include besides carbon atoms 1, 2, 3 or 4 heteroatoms selected from the group of N, O and S”, refers to, for example,
a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl; or
a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

The term “two radicals R^{c} that are bound to adjacent ring member atoms form together with said ring member atoms a fused cycle” refers to a condensed bicyclic ring system, wherein 5- or 6-membered heteroaryl carries a fused-on 5-, 6- or 7-membered carbocyclic or heterocyclic ring it being possible that these rings are saturated or partially saturated or aromatic.

The term “one or two CH₂ groups of the abovementioned cycles may be respectively replaced by one or two C(═O) or C(═S) groups” refers to an exchange of carbon atoms from a saturated or partially unsaturated 3-, 4-, 5-, 6- or 7-membered carbocycle or a saturated or partially unsaturated 3-, 4-, 5-, 6- or 7-membered heterocycle, resulting in cycles such as cyclopropanone, cyclopentanone, cyclopropanethione, cyclopentanethione, 5-oxazolone, cyclohexane-1,4-dione, cyclohexane-1,4-dithione, cyclohex-2-ene-1,4-dione or cyclohex-2-ene-1,4-dithione.

As regards the fungicidal activity of the compounds I, preference is given to those compounds I wherein the substituents and variables (e.g. R^{a6}, R^{a6}, R, R¹, R², R³, R⁴, R^{b}, R^{c}, R′, R″, R′″, R^{A}, R^{B}, n and Het) have independently of each other or more preferably in combination the following meanings and the groups mentioned herein for a substituent or for a combination of substituents are furthermore, independently of the combination in which they are mentioned, a particularly preferred embodiment of the substituent or of the combination of substituents in question.

According to one embodiment of the present invention R^{a5} and R^{a6} independently of each other are selected from hydrogen, halogen, CN, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkoxy-C₁-C₄-alkyl, C₁-C₄-alkoxy-C₁-C₄-alkoxy, C₂-C₄-alkenyl, C₂-C₄-alkynyl, C₃-C₈-cycloalkyl, C₃-C₈-cycloalkyloxy and (C₁-C₄-alkoxy)carbonyl.

In another embodiment R^{a5} and R^{a6} independently of each other are selected from hydrogen, halogen, CN, C₁-C₄-alkyl, C₁-C₄-haloalkyl, C₁-C₄-alkoxy, C₁-C₄-haloalkoxy, C₁-C₄-alkoxy-C₁-C₄-alkyl, C₁-C₄-alkoxy-C₁-C₄-alkoxy and (C₁-C₄-alkoxy)carbonyl.

In a further embodiment R^{a5} and R^{a6} independently of each other are selected from halogen, CN, C₁-C₄-alkyl, C₁-C₄-haloalkyl, C₁-C₄-alkoxy, C₁-C₄-haloalkoxy, C₁-C₄-alkoxy-C₁-C₄-alkyl, C₁-C₄-alkoxy-C₁-C₄-alkoxy and (C₁-C₄-alkoxy)carbonyl.

In another aspect R^{a5} and R^{a6} independently of each other are selected from halogen, CN, C₁-C₄-alkyl, C₁-C₄-haloalkyl, C₁-C₄-alkoxy, C₁-C₄-haloalkoxy, C₁-C₄-alkoxy-C₁-C₄-alkyl, C₁-C₄-alkoxy-C₁-C₄-alkoxy or (C₁-C₄-alkoxy)carbonyl, and it being possible that one of R^{a5} or R^{a6} can in addition be hydrogen.

In a further embodiment R^{a5} and R^{a6} independently of each other are selected from hydrogen, Cl, F, CH₃, CH₂CH₃, OCH₃, OCF₃, CH₂OCH₃, CN, OCH₂OCH₃, CF₃, CHFCH₃, COOCH₃ and COOCH₂CH₃.

In another embodiment R^{a5} and R^{a6} independently of each other are selected from Cl, F, CH₃, CH₂CH₃, OCH₃, OCF₃, CH₂OCH₃, CN, OCH₂OCH₃, CF₃, CHFCH₃, COOCH₃ and COOCH₂CH₃, and it being possible that one of R^{a5} or R^{a6} can in addition be hydrogen.

In a further embodiment R^{a5} and R^{a6} independently of each other are selected from Cl, F, CH₃, OH₂CH₃, OCH₃, OCF₃, CH₂OCH₃, CN, OCH₂OCH₃, CF₃, CHFCH₃, COOCH₃ and COOCH₂CH₃.

In yet another embodiment R^{a5} and R^{a6} independently of each other are selected from hydrogen, Cl, CH₃, CH₂CH₃, CH₂OCH₃, OCH₃, CN, CHFCH₃, COOCH₂H₃ and COOCH₃.

In still a further embodiment R^{a5} and R^{a6} independently of each other are selected from Cl, CH₃, CH₂CH₃, CH₂OCH₃, OCH₃, CN, CHFCH₃, COOCH₂H₃ and COOCH₃, and it being possible that one of R^{a5} or R^{a6} can in addition be hydrogen.

In a preferred embodiment R^{a5} and R^{a6} independently of each other are selected from Cl, CH₃, CH₂CH₃, CH₂OCH₃, OCH₃, CHFCH₃, COOCH₂H₃ and COOCH₃.

In a further embodiment R^{a5} and R^{a6} independently of each other are selected from hydrogen, halogen, OH, CN, C₁-C₄-alkyl, C₂-C₄-alkenyl, C₁-C₄-haloalkyl, C₁-C₄-alkoxy and (C₁-C₄-alkoxy)carbonyl; or R^{a5} and R^{a6} together with two ring member carbon atoms to which they are attached, form a fused 5- or 6-membered saturated, partially unsaturated or aromatic carbocycle or heterocycle, wherein the ring member atoms of the fused heterocycle include besides carbon atoms 1, 2 or 3 heteroatoms selected from the group of N, O and S, and wherein the fused carbocycle or heterocycle is unsubstituted or carries 1, 2, 3 or 4 identical or different radicals selected from halogen, CN, C₁-C₄-alkyl, C₁-C₄-alkoxy, C₁-C₄-haloalkyl and C₁-C₄-haloalkoxy.

In still another embodiment R^{a5} and R^{a6} independently of each other are selected from hydrogen, halogen, OH, CN, C₁-C₄-alkyl, C₂-C₄-alkenyl, C₁-C₄-haloalkyl, C₁-C₄-alkoxy and (C₁-C₄-alkoxy)carbonyl; or R^{a5} and R^{a6} together with two ring member carbon atoms to which they are attached, form a fused 5- or 6-membered aromatic carbocycle or heterocycle, wherein the ring member atoms of the fused heterocycle include besides carbon atoms 1, 2 or 3 heteroatoms selected from the group of N, O and S, and wherein the fused carbocycle or heterocycle is unsubstituted or carries 1, 2, 3 or 4 identical or different radicals selected from halogen, CN, C₁-C₄-alkyl, C₁-C₄-alkoxy, C₁-C₄-haloalkyl and C₁-C₄-haloalkoxy.

In still another embodiment R^{a5} and R^{a6} independently of each other are selected from halogen, OH, CN, C₁-C₄-alkyl, C₂-C₄-alkenyl, C₁-C₄-haloalkyl, C₁-C₄-alkoxy and (C₁-C₄-alkoxy)carbonyl; and it being possible that one of R^{a5} or R^{a6} can in addition be hydrogen; or R^{a5} and R^{a6} together with two ring member carbon atoms to which they are attached, form a fused 5- or 6-membered aromatic carbocycle or heterocycle, wherein the ring member atoms of the fused heterocycle include besides carbon atoms 1, 2 or 3 heteroatoms selected from the group of N, O and S, and wherein the fused carbocycle or heterocycle is unsubstituted or carries 1, 2, 3 or 4 identical or different radicals selected from halogen, CN, C₁-C₄-alkyl, C₁-C₄-alkoxy, C₁-C₄-haloalkyl and C₁-C₄-haloalkoxy.

In one embodiment R^{a5} is hydrogen. In one embodiment R^{a5} is CI. In another embodiment R^{a5} is F. In a further embodiment R^{a5} is CH₃. In another embodiment R^{a5} is CH₂CH₃. In still another embodiment R^{a5} is CH₂OCH₃. In yet another embodiment R^{a5} is OCH₃. In still a further embodiment R^{a5} is CO₂CH₃. In one embodiment R^{a5} is CO₂CH₂CH₃.

In one embodiment R^{a6} is hydrogen. In one embodiment R^{a6} is CI. In another embodiment R^{a6} is F. In still another embodiment R^{a6} is CH₃. In another embodiment R^{a6} is CH₂CH₃. In yet another embodiment R^{a6} is CH₂CH₃. In a further embodiment R^{a6} is OCH₃. In one embodiment R^{a6} is CO₂CH₃. In another aspect R^{a6} is CO₂CH₂CH₃. In yet another aspect R^{a6} is CHFCH₃.

Further preferred embodiments relate to compounds I wherein R^{a5} and R^{a6} in each case constitute together with two ring member carbon atoms of the pyrimidine ring one of the following heterocyclic groups as defined in line A.1-1 to line A.1-26 in table A.1, wherein #5 and #6 indicate the point of attachment to the pyrimidine ring, each respectively corresponding to the positions of either substituent R^{a5} or R^{a6}.

TABLE A.1

| line | R^{a5}/R^{a6} |
|---|---|
| A.1-1 | #5—CH═CH—CH═CH—#6 |
| A.1-2 | #5—CH₂—CH₂—CH₂—CH₂—#6 |
| A.1-3 | #5—CH═CH—CH═N—#6 |
| A.1-4 | #5—N═CH—CH═CH—#6 |
| A.1-5 | #5—CH═N—CH═N—#6 |
| A.1-6 | #5—N═CH—N═CH—#6 |
| A.1-7 | #5—CH₂—CH₂—CH₂—#6 |
| A.1-8 | #5—N═CH—CH═N—#6 |
| A.1-9 | #5—O—CH₂—O—#6 |
| A.1-10 | #5—NH—CH═N—#6 |
| A.1-11 | #5—S—CH═N—#6 |
| A.1-12 | #5—N═CH—S—#6 |
| A.1-13 | #5—O—CH═N—#6 |
| A.1-14 | #5—N═CH—O—#6 |
| A.1-15 | #5—O—CH═CH—#6 |
| A.1-16 | #5—S—CH═CH—#6 |
| A.1-17 | #5—O—N═CH—#6 |
| A.1-18 | #5—S—N═CH—#6 |
| A.1-19 | #5—CH═N—O—#6 |
| A.1-20 | #5—CH═N—S—#6 |
| A.1-21 | #5—N(CH₃)—CH═CH—#6 |
| A.1-22 | #5—CH═CH—N(CH₃)—#6 |
| A.1-23 | #5═CH—N(NH₂)—N═#6 |
| A.1-24 | #5—CH═N—N(CH₃)—#6 |
| A.1-25 | #5═N—N(CH₃)—CH═#6 |
| A.1-26 | #5—N(CH₃)—N═CH—#6 |

Further preferred embodiments relate to compounds I wherein R^{a5} and R^{a6} in each case are one of the following combinations of R^{a5} and R^{a6} as defined in line A.2-1 to line A.2-16 in table A.2.

TABLE A.2

| Line | R^{a5} | R^{a6} |
|---|---|---|
| A.2-1 | CH₃ | Cl |
| A.2-2 | OCH₃ | Cl |
| A.2-3 | OCH₃ | COOCH₃ |
| A.2-4 | OCH₃ | CH₃ |
| A.2-5 | Cl | CH₃ |
| A.2-6 | Cl | CH₂CH₃ |
| A.2-7 | COOCH₂CH₃ | CH₂CH₃ |
| A.2-8 | CH₃ | COOCH₃ |
| A.2-9 | COOCH₃ | CH₂CH₃ |
| A.2-10 | OCH₃ | CH₂OCH₃ |
| A.2-11 | Cl | CHFCH₃ |
| A.2-12 | OCH₃ | CHFCH₃ |

| | |
|---|---|
| A.2-13 | as defined for A.1-3 |
| A.2-14 | as defined for A.1-4 |
| A.2-15 | as defined for A.1-8 |
| A.2-16 | as defined for A.1-12 |

In the compounds I according to the invention, R^{A}, R^{B} in radical R^{a5} preferably is hydrogen, C₁-C₄-alkyl.

In the compounds I according to the invention, R^{A}, R^{B} in radical R^{a6} preferably is hydrogen, C₁-C₄-alkyl.

In the compounds I according to the invention, R′ in radical R^{a5} preferably is hydrogen, NH₂, C₁-C₄-alkyl, C₁-C₄-alkoxy.

In the compounds I according to the invention, R′ in radical R^{a6} preferably is hydrogen, NH₂, C₁-C₄-alkyl, C₁-C₄-alkoxy.

In the compounds I according to the invention, R″ in radical R^{a5} preferably is hydrogen, C₁-C₄-alkyl.

In the compounds I according to the invention, R″ in radical R^{a6} preferably is hydrogen, C₁-C₄-alkyl.

In the compounds I according to the invention, R′″ in radical R^{a5} preferably is hydrogen.

In the compounds I according to the invention, R′″ in radical R^{a6} preferably is hydrogen.

In the compounds I according to the invention, R is hydrogen, C₁-C₄-alkyl, C₁-C₄-alkoxy, C₁-C₄-alkoxy-C₁-C₄-alkyl, C₂-C₄-alkenyl, C₂-C₄-alkynyl, CN, CH₂CN and CH₂—O—C(═O)R′, wherein R′ is hydrogen, C₁-C₄-alkyl, C₁-C₄-alkoxy; more preferably R is hydrogen, C₁-C₄-alkyl, C₁-C₄-alkoxy-C₁-C₄-alkyl, C₂-C₄-alkenyl and C₂-C₄-alkynyl; in another preferred embodiment R is hydrogen, C₁-C₄-alkyl, C₂-C₄-alkenyl or C₂-C₄-alkynyl; in another preferred embodiment R is hydrogen or C₁-C₄-alkyl; more preferably R is hydrogen; in another more preferred embodiment R is CH₃.

In the compounds I according to the invention, R³ and R⁴ independently of each other are selected from hydrogen, CN, C₁-C₄-hydroxyalkyl, C₁-C₄-alkyl, C₂-C₄-alkenyl, C₂-C₄-alkynyl and C₃-C₈-cycloalkyl; in another embodiment the group —CR³R⁴— is —CH₂—, —CH(CH₃)—, —CH(CH₂CH₃)—, —C(CH₃)₂—, —CHCN—, —CH(C(═O)—C₁-C₄-alkoxy)-, —CH(C(═O)NH₂)—, —C(═O)N(C₁-C₄-alkyl)₂- or —CH(C(═O)OH)—. In another preferred embodiment the group —CR³R⁴— is —CH(CH₃)—, —CH(CH₂CH₃)—, —C(CH₃)₂—, —CHCN— or —CH(C(═O)—C₁-C₄-alkoxy).

In still another embodiment the group —CR³R⁴— is —CH(CH₃)—, —C(CH₃)₂—, —CH(C₂H₅)— or —CHCN—. In one aspect the group —CR³R⁴— is —CH(CH₃)—. In a further aspect the group —CR³R⁴— is —C(CH₃)₂—. In one embodiment the group —CR³R⁴— is —CH(C₂H₅)—. In one embodiment the group —CR³R⁴— is —CHCN—. In another preferred embodiment the group —CR³R⁴— is —CH(C(═O)—C₁-C₄-alkoxy). In a further preferred embodiment the group —CR³R⁴— is —H(C(═O)—OCH₃). In still a further preferred embodiment the group —CR³R⁴— is —CH(C(═O)—OCH₂CH₃).

In one aspect of the invention two radicals R³ and R⁴ that are bound to the same carbon atom form together with said carbon atom a carbocycle or heterocycle selected from cyclopropyl, cyclobutyl, cyclopentyl, cyclopentenyl, cyclopentadienyl, cyclohexyl, cyclohexenyl, oxirane, aziridine, thiirane, oxetane, azetidine, thiethane, 2-tetrahydrofuranyl, 3-tetrahydrofuranyl, 2-tetrahydrothienyl, 3-tetrahydrothienyl, 2-pyrrolidinyl and 3-pyrrolidinyl; and wherein the abovementioned cycle is unsubstituted or carries 1, 2, 3 or 4 substituents selected from halogen, CN, OH, SH, C₁-C₄-alkyl, C₁-C₄-alkoxy or C₁-C₄-alkylthio; and one or two CH₂ groups of the abovementioned cycles may be respectively replaced by one or two C(═O) or C(═S) groups;

In another aspect of the invention two radicals R³ and R⁴ that are bound to the same carbon atom form together with said carbon atom a carbocycle or heterocycle selected from cyclopropyl, cyclobutyl, cyclopentyl, cyclohexyl, oxirane, aziridine, thiirane, oxetane, azetidine2-tetrahydrofuranyl and 3-tetrahydrofuranyl; and wherein the abovementioned cycle is unsubstituted or carries 1, 2, 3 or 4 substituents selected from halogen, CN, OH, SH, C₁-C₄-alkyl, C₁-C₄-alkoxy or C₁-C₄-alkylthio.

In the compounds I according to the invention, R¹ and R² independently of each other are preferably selected from hydrogen, CN, C₁-C₄-alkyl, C₁-C₄-haloalkyl, C₁-C₄-alkoxy, C₁-C₄-haloalkoxy and C₃-C₈-cycloalkyl; in one aspect R¹ and R² independently of each other are selected from hydrogen, CN, CH₃, CH₂CH₃, F, Cl or OCH₃; in another aspect R¹ and R² independently of each other are selected from hydrogen or C₁-C₄-alkyl; in another more preferred embodiment R¹ and R² independently of each other are hydrogen or CH₃; more preferably R¹ and R² are hydrogen.

Further preferred embodiments relate to compounds I wherein R³, R⁴, n, R^{b} in each case are one of the following combinations B-1 to B-12 in table B, and wherein the position of R^{b} on the phenyl ring is defined relative to the alkyne-moiety bound to the phenyl ring as being in ortho (o-) position; n=0 indicates that no substituent R^{b} is present on the phenyl ring:

TABLE B

| line | R³ | R⁴ | n | R^{b} |
|---|---|---|---|---|
| B-1 | H | H | 0 | |
| B-2 | H | CH₃ | 0 | |
| B-3 | CH₃ | CH₃ | 0 | |
| B-4 | H | COOCH₃ | 0 | |
| B-5 | H | COOCH₂CH₃ | 0 | |
| B-6 | H | CN | 0 | |
| B-7 | H | H | 1 | o-F |
| B-8 | H | CH₃ | 1 | o-F |
| B-9 | CH₃ | CH₃ | 1 | o-F |
| B-10 | H | COOCH₃ | 1 | o-F |
| B-11 | H | COOCH₂CH₃ | 1 | o-F |
| B-12 | H | CN | 1 | o-F |

In the compounds I according to the invention R^{b} are independently selected from halogen, CN, NO₂, C₁-C₄-alkyl, C₁-C₄-haloalkyl, C₁-C₄-alkoxy, C₁-C₄-haloalkoxy, C₁-C₄-alkoxy-C₁-C₄-alkyl and (C₁-C₄-alkoxy)carbonyl; more preferably R^{b} are independently selected from halogen, CN, C₁-C₄-alkyl, C₁-C₄-haloalkyl, C₁-C₄-alkoxy and (C₁-C₄-alkoxy)carbonyl; in another preferred embodiment R^{b} are independently selected from halogen and C₁-C₄-alkoxy; most preferably R^{b} are independently selected from halogen, CN, CH₃, CF₃ and OCH₃. In a particularly preferred embodiment R^{b} is F. In another particularly preferred embodiment R^{b} is CH₃. In a further particularly preferred embodiment R^{b} is CF₃. In yet another particularly preferred embodiment R^{b} is OCH₃. In still a further embodiment R^{b} is attached to the phenyl ring adjacent (in ortho-position) to the alkyne group. A further embodiment relates to compounds I wherein R^{b} is attached in meta-position to the alkyne group.

In the compounds I according to the invention, n is preferably 0.

A further embodiment relates to compounds I wherein n is preferably 1.

A further embodiment relates to compounds I wherein n is preferably 0 or 1.

A further embodiment relates to compounds I wherein n is preferably 2.

A further embodiment relates to compounds I wherein n is preferably 0, 1 or 2.

A further embodiment relates to compounds I wherein n is preferably 3.

A further embodiment relates to compounds I wherein n is preferably 0, 1, 2 or 3.

A further embodiment relates to compounds I wherein n is preferably 4.

In the compounds I according to the invention, Het is pyrimidin-2-yl, pyrimidin-3-yl, pyrimidin-4-yl, pyridin-2-yl, pyridin-3-yl, pyridin-4-yl, thiazol-2-yl, thiazol-4-yl, thiazol-5-yl, isothiazol-3-yl, isothiazol-4-yl, isothiazol-5-yl, pyrazin-2-yl, pyridazin-3-yl, 1,3,5-triazin-2-yl or 1,2,4-triazin-3-yl; more preferably Het is pyrimidin-2-yl, pyrimidin-3-yl, pyrimidin-4-yl, pyridin-2-yl, pyridin-3-yl, pyridin-4-yl, thiazol-2-yl, pyrazin-2-yl, pyridazin-3-yl, 1,3,5-triazin-2-yl or 1,2,4-triazin-3-yl; more preferably Het is pyrimidin-2-yl, pyrimidin-3-yl, pyrimidin-4-yl, pyridin-2-yl, pyridin-3-yl, pyridin-4-yl or thiazol-2-yl; in another aspect Het is pyrimidin-2-yl, pyridin-2-yl, pyridin-3-yl or pyridin-4-yl.

According to one embodiment Het is unsubstituted; in a further embodiment Het is unsubstituted or substituted by 1 radical R^{c} as defined or preferably defined herein. In a further embodiment Het is substituted by 1 radical R^{c} as defined or preferably defined herein.

In still a further embodiment Het is unsubstituted or substituted by 1 or 2 independently selected radicals R^{c} as defined or preferably defined herein. In another embodiment Het is substituted by 1 or 2 independently selected radicals R^{c} as defined or preferably defined herein. In yet another preferred Het is unsubstituted or substituted by 1, 2 or 3 independently selected radicals R^{c} as defined or preferably defined herein. In still another embodiment Het is unsubstituted or substituted by 1, 2, 3, or 4 independently selected radicals R^{c} as defined or preferably defined herein.

In a further preferred embodiment Het is a pyridinyl or pyrimidinyl ring wherein the pyridinyl or pyrimidinyl are unsubstituted or carry 1 or 2 groups R^{c} as defined or preferably defined herein. In one aspect of the invention Het is pyridinyl, more preferably pyridin-2-yl; in particular pyridine-2-yl, which is substituted with one substituent R^{c} as defined or preferably defined herein; in a particular embodiment thereof R^{c} is CF₃.

In the compounds I according to the invention, R^{c} are preferably independently selected from halogen, CN, C₁-C₆-alkyl, C₁-C₆-haloalkyl, C₁-C₆-alkoxy, C₁-C₆-haloalkoxy, C₂-C₆-alkenyl, C₂-C₆-alkynyl, C(═O)R′, C(═NOR″)R′″, C₃-C₈-cycloalkyl, phenyl and phenoxy. In another embodiment R^{c} are independently selected from halogen, C₁-C₆-alkyl, C₁-C₆-haloalkyl, C₁-C₄-alkoxy and (C₁-C₄-alkoxy)carbonyl. In a further embodiment R^{c} are independently selected from F, Cl, Br, CN, C₁-C₂-alkylsulfonyl, C₁-C₂-alkoxycarbonyl, aminocarbonyl, C₁-C₂-alkylaminocarbonyl, di(C₁-C₂-alkyl)aminocarbonyl, C₁-C₂-alkoxy, CF₃, CHF₂, OCF₃ and OCHF₂. In a further embodiment R^{c} are independently selected from F, Cl, CN, CH₃, OCH₃, CF₃, CHF₂, OCF₃, OCHF₂ and COOCH₃. Preferably R^{c} are independently selected from F, Cl, CN and CF₃; in particular R^{c} is CF₃.

Preferred embodiments of the invention relate to compounds I, in which the group Het is one of the following radicals H-1 to H-38 in table H:

TABLE H

| No. | Het |
|---|---|
| H-1 | 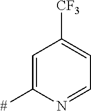 |
| H-2 | 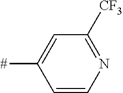 |
| H-3 | 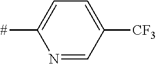 |
| H-4 | 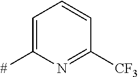 |
| H-5 | 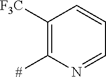 |
| H-6 | 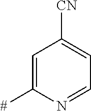 |
| H-7 | 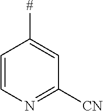 |
| H-8 | 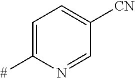 |
| H-9 | 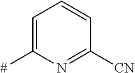 |
| H-10 | 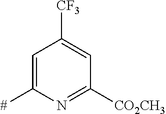 |
| H-11 | 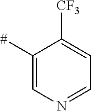 |
| H-12 | 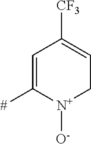 |
| H-13 | 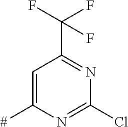 |
| H-14 | 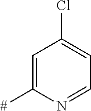 |
| H-15 | 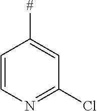 |
| H-16 | 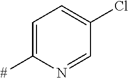 |
| H-17 | 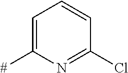 |
| H-18 | 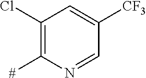 |
| H-19 | 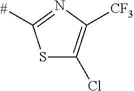 |
| H-20 | 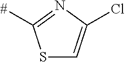 |
| H-21 | 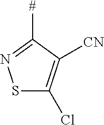 |
| H-22 | 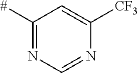 |
| H-23 | 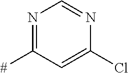 |
| H-24 | 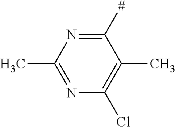 |
| H-25 | 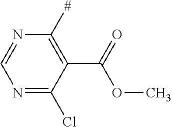 |
| H-26 | 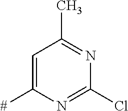 |
| H-27 | 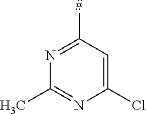 |
| H-28 | 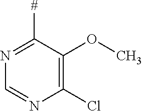 |
| H-29 | 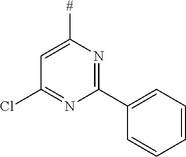 |
| H-30 | 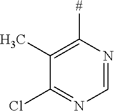 |
| H-31 | 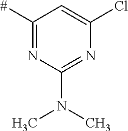 |
| H-32 | 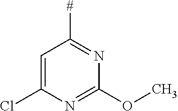 |
| H-33 | 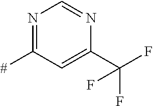 |
| H-34 | 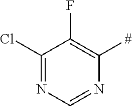 |
| H-35 | 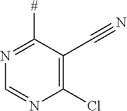 |
| H-36 | 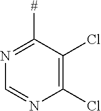 |
| H-37 | 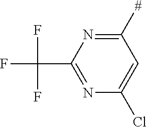 |
| H-38 | 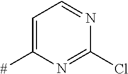 |

in which # indicates the point of attachment.

According to a further embodiment, the present invention relates to compounds of the formula I wherein:
R^{a5}, R^{a6} independently of each other are halogen, OH, CN, C₁-C₄-alkyl, C₂-C₄-alkenyl, C₁-C₄-haloalkyl, C₁-C₄-alkoxy, C₁-C₄-alkoxy-C₁-C₄-alkyl or (C₁-C₄-alkoxy)carbonyl; and it being possible that one of R^{a5} or R^{a6} can in addition be hydrogen; or
R^{a5} and R⁶ together with two ring member carbon atoms to which they are attached, form a fused 5- or 6-membered saturated, partially unsaturated or aromatic carbocycle or heterocycle, wherein the ring member atoms of the fused heterocycle include besides carbon atoms 1, 2 or 3 heteroatoms selected from N, O and S, and wherein the fused carbocycle or heterocycle is unsubstituted or carries 1, 2, 3 or 4 identical or different radicals selected from halogen, CN, C₁-C₄-alkyl, C₁-C₄-alkoxy, C₁-C₄-haloalkyl and C₁-C₄-haloalkoxy;
R is hydrogen, C₁-C₄-alkyl, C₂-C₄-alkenyl or C₂-C₄-alkynyl;
R³ and R⁴ independently of each other are hydrogen, CN, C₁-C₄-hydroxyalkyl, C₁-C₄-alkyl, C₂-C₄-alkenyl, C₂-C₄-alkynyl or C₃-C₈-cycloalkyl;
R¹, R² are independently selected from hydrogen and C₁-C₄-alkyl;
n is 0 or 1;
R^{b} is halogen or C₁-C₄-alkoxy;
Het is a pyridinyl or pyrimidinyl ring; and wherein the pyridinyl or pyrimidinyl are unsubstituted or carry 1 or 2 groups R^{c}; wherein
   R^{c} are independently selected from halogen, C₁-C₆-alkyl, C₁-C₆-haloalkyl, C₁-C₄-alkoxy and (C₁-C₄-alkoxy)carbonyl;

and the N-oxides and the agriculturally acceptable salts of the compounds of formula I.

According to a further embodiment, the present invention relates to compounds of the formula I wherein:
R^{a5}, R^{a6} independently of each other are halogen, OH, CN, C₁-C₄-alkyl, C₂-C₄-alkenyl, C₁-C₄-haloalkyl, C₁-C₄-alkoxy, C₁-C₄-alkoxy-C₁-C₄-alkyl or (C₁-C₄-alkoxy)carbonyl; and it being possible that one of R^{a5} or R^{a6} can in addition be hydrogen;
R is hydrogen, C₁-C₄-alkyl, C₂-C₄-alkenyl or C₂-C₄-alkynyl;
R³, R⁴ are independently selected from hydrogen and C₁-C₄-alkyl;
R¹, R² are independently selected from hydrogen and C₁-C₄-alkyl;
n is 0 or 1;
R^{b} is halogen;
Het is a pyridinyl or pyrimidinyl ring; and wherein the pyridinyl or pyrimidinyl are unsubstituted or carry 1 or 2 groups R^{c}; wherein
   R^{c} are independently selected from halogen, C₁-C₆-alkyl, C₁-C₆-haloalkyl, C₁-C₄-alkoxy and (C₁-C₄-alkoxy)carbonyl;

and the N-oxides and the agriculturally acceptable salts of the compounds of formula I.

With respect to their use, particular preference is given to the compounds I.A or I.B.

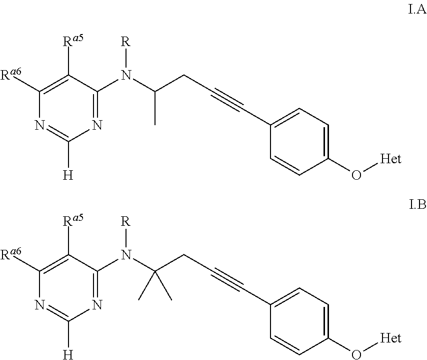

A skilled person will readily understand that the preferences given in connection with compounds of formula I also apply for formulae I.A or I.B as defined herein.

According to a further embodiment, the present invention relates to compounds of the formula I.A or I.B, wherein:
R^{a5}, R^{a6} independently of each other are halogen, OH, CN, C₁-C₄-alkyl, C₂-C₄-alkenyl, C₁-C₄-haloalkyl, C₁-C₄-alkoxy, C₁-C₄-alkoxy-C₁-C₄-alkyl or (C₁-C₄-alkoxy)carbonyl; and it being possible that one of R^{a5} or R^{a6} can in addition be hydrogen; or
R^{a5} and R⁶ together with two ring member carbon atoms to which they are attached, form a fused 5- or 6-membered saturated, partially unsaturated or aromatic carbocycle or heterocycle, wherein the ring member atoms of the fused heterocycle include besides carbon atoms 1, 2 or 3 heteroatoms selected from N, O and S, and wherein the fused carbocycle or heterocycle is unsubstituted or carries 1, 2, 3 or 4 identical or different radicals selected from halogen, CN, C₁-C₄-alkyl, C₁-C₄-alkoxy, C₁-C₄-haloalkyl and C₁-C₄-haloalkoxy;
R is hydrogen, C₁-C₄-alkyl, C₂-C₄-alkenyl or C₂-C₄-alkynyl;
Het is a pyridinyl or pyrimidinyl ring; and wherein the pyridinyl or pyrimidinyl are unsubstituted or carry 1 or 2 groups R^{c}; wherein
   R^{c} are independently selected from halogen, C₁-C₆-alkyl, C₁-C₆-haloalkyl, C₁-C₄-alkoxy and (C₁-C₄-alkoxy)carbonyl;

and the N-oxides and the agriculturally acceptable salts of the compounds of formula I.A or I.B.

According to a further embodiment, the present invention relates to compounds of the formula I.A or I.B, wherein:
R^{a5}, R^{a6} independently of each other are halogen, OH, CN, C₁-C₄-alkyl, C₂-C₄-alkenyl, C₁-C₄-haloalkyl, C₁-C₄-alkoxy, C₁-C₄-alkoxy-C₁-C₄-alkyl or (C₁-C₄-alkoxy)carbonyl; and it being possible that one of R^{a5} or R^{a6} can in addition be hydrogen;
R is hydrogen, C₁-C₄-alkyl, C₂-C₄-alkenyl or C₂-C₄-alkynyl;
Het is a pyridinyl or pyrimidinyl ring; and wherein the pyridinyl or pyrimidinyl are unsubstituted or carry 1 or 2 groups R^{c}; wherein
   R^{c} are independently selected from halogen, C₁-C₆-alkyl, C₁-C₆-haloalkyl, C₁-C₄-alkoxy and (C₁-C₄-alkoxy)carbonyl;

and the N-oxides and the agriculturally acceptable salts of the compounds of formula I.A or I.B.

Further preferred embodiments relate to compounds I as defined in Table C below wherein R, R¹ and R² in each case are hydrogen and the meaning of the radicals R^{a5}, R^{a6}, R³, R⁴, n, R^{b} and Het in each case are one of the combinations as defined in lines C-1 to C-960 in Table C (compound I.c-1 to compound I.c-960); and wherein the meaning of the combination of substituents R^{a5} and R^{a6} in each case are one of the combinations selected from one line A.2-1 to line A.2-16 in table A.2, and wherein the meaning of R³, R⁴, n and R^{b} in each case are one of the combinations selected from one line B-1 to B-12 in table B and wherein the group Het in each case is one of the radicals selected from H-1, H-3, H-14, H-16 and H-23 as defined in table H.

TABLE C

| line | R^{a5}, R^{a6} | R³, R⁴, n, R^{b} | Het |
|---|---|---|---|
| C-1 | A.2-1 | B-1 | H-1 |
| C-2 | A.2-2 | B-1 | H-1 |
| C-3 | A.2-3 | B-1 | H-1 |
| C-4 | A.2-4 | B-1 | H-1 |
| C-5 | A.2-5 | B-1 | H-1 |
| C-6 | A.2-6 | B-1 | H-1 |
| C-7 | A.2-7 | B-1 | H-1 |
| C-8 | A.2-8 | B-1 | H-1 |
| C-9 | A.2-9 | B-1 | H-1 |
| C-10 | A.2-10 | B-1 | H-1 |
| C-11 | A.2-11 | B-1 | H-1 |
| C-12 | A.2-12 | B-1 | H-1 |
| C-13 | A.2-13 | B-1 | H-1 |
| C-14 | A.2-14 | B-1 | H-1 |
| C-15 | A.2-15 | B-1 | H-1 |
| C-16 | A.2-16 | B-1 | H-1 |
| C-17 | A.2-1 | B-2 | H-1 |
| C-18 | A.2-2 | B-2 | H-1 |
| C-19 | A.2-3 | B-2 | H-1 |
| C-20 | A.2-4 | B-2 | H-1 |
| C-21 | A.2-5 | B-2 | H-1 |
| C-22 | A.2-6 | B-2 | H-1 |
| C-23 | A.2-7 | B-2 | H-1 |
| C-24 | A.2-8 | B-2 | H-1 |
| C-25 | A.2-9 | B-2 | H-1 |
| C-26 | A.2-10 | B-2 | H-1 |
| C-27 | A.2-11 | B-2 | H-1 |
| C-28 | A.2-12 | B-2 | H-1 |
| C-29 | A.2-13 | B-2 | H-1 |
| C-30 | A.2-14 | B-2 | H-1 |
| C-31 | A.2-15 | B-2 | H-1 |
| C-32 | A.2-16 | B-2 | H-1 |
| C-33 | A.2-1 | B-3 | H-1 |
| C-34 | A.2-2 | B-3 | H-1 |
| C-35 | A.2-3 | B-3 | H-1 |
| C-36 | A.2-4 | B-3 | H-1 |
| C-37 | A.2-5 | B-3 | H-1 |
| C-38 | A.2-6 | B-3 | H-1 |
| C-39 | A.2-7 | B-3 | H-1 |
| C-40 | A.2-8 | B-3 | H-1 |
| C-41 | A.2-9 | B-3 | H-1 |
| C-42 | A.2-10 | B-3 | H-1 |
| C-43 | A.2-11 | B-3 | H-1 |
| C-44 | A.2-12 | B-3 | H-1 |
| C-45 | A.2-13 | B-3 | H-1 |
| C-46 | A.2-14 | B-3 | H-1 |
| C-47 | A.2-15 | B-3 | H-1 |
| C-48 | A.2-16 | B-3 | H-1 |
| C-49 | A.2-1 | B-4 | H-1 |
| C-50 | A.2-2 | B-4 | H-1 |
| C-51 | A.2-3 | B-4 | H-1 |
| C-52 | A.2-4 | B-4 | H-1 |
| C-53 | A.2-5 | B-4 | H-1 |
| C-54 | A.2-6 | B-4 | H-1 |
| C-55 | A.2-7 | B-4 | H-1 |
| C-56 | A.2-8 | B-4 | H-1 |
| C-57 | A.2-9 | B-4 | H-1 |
| C-58 | A.2-10 | B-4 | H-1 |
| C-59 | A.2-11 | B-4 | H-1 |
| C-60 | A.2-12 | B-4 | H-1 |
| C-61 | A.2-13 | B-4 | H-1 |
| C-62 | A.2-14 | B-4 | H-1 |
| C-63 | A.2-15 | B-4 | H-1 |
| C-64 | A.2-16 | B-4 | H-1 |
| C-65 | A.2-1 | B-5 | H-1 |
| C-66 | A.2-2 | B-5 | H-1 |
| C-67 | A.2-3 | B-5 | H-1 |
| C-68 | A.2-4 | B-5 | H-1 |
| C-69 | A.2-5 | B-5 | H-1 |
| C-70 | A.2-6 | B-5 | H-1 |
| C-71 | A.2-7 | B-5 | H-1 |
| C-72 | A.2-8 | B-5 | H-1 |
| C-73 | A.2-9 | B-5 | H-1 |
| C-74 | A.2-10 | B-5 | H-1 |
| C-75 | A.2-11 | B-5 | H-1 |
| C-76 | A.2-12 | B-5 | H-1 |
| C-77 | A.2-13 | B-5 | H-1 |
| C-78 | A.2-14 | B-5 | H-1 |
| C-79 | A.2-15 | B-5 | H-1 |
| C-80 | A.2-16 | B-5 | H-1 |
| C-81 | A.2-1 | B-6 | H-1 |
| C-82 | A.2-2 | B-6 | H-1 |
| C-83 | A.2-3 | B-6 | H-1 |
| C-84 | A.2-4 | B-6 | H-1 |
| C-85 | A.2-5 | B-6 | H-1 |
| C-86 | A.2-6 | B-6 | H-1 |
| C-87 | A.2-7 | B-6 | H-1 |
| C-88 | A.2-8 | B-6 | H-1 |
| C-89 | A.2-9 | B-6 | H-1 |
| C-90 | A.2-10 | B-6 | H-1 |
| C-91 | A.2-11 | B-6 | H-1 |
| C-92 | A.2-12 | B-6 | H-1 |
| C-93 | A.2-13 | B-6 | H-1 |
| C-94 | A.2-14 | B-6 | H-1 |
| C-95 | A.2-15 | B-6 | H-1 |
| C-96 | A.2-16 | B-6 | H-1 |
| C-97 | A.2-1 | B-7 | H-1 |
| C-98 | A.2-2 | B-7 | H-1 |
| C-99 | A.2-3 | B-7 | H-1 |
| C-100 | A.2-4 | B-7 | H-1 |
| C-101 | A.2-5 | B-7 | H-1 |
| C-102 | A.2-6 | B-7 | H-1 |
| C-103 | A.2-7 | B-7 | H-1 |
| C-104 | A.2-8 | B-7 | H-1 |
| C-105 | A.2-9 | B-7 | H-1 |
| C-106 | A.2-10 | B-7 | H-1 |
| C-107 | A.2-11 | B-7 | H-1 |
| C-108 | A.2-12 | B-7 | H-1 |
| C-109 | A.2-13 | B-7 | H-1 |
| C-110 | A.2-14 | B-7 | H-1 |
| C-111 | A.2-15 | B-7 | H-1 |
| C-112 | A.2-16 | B-7 | H-1 |
| C-113 | A.2-1 | B-8 | H-1 |
| C-114 | A.2-2 | B-8 | H-1 |
| C-115 | A.2-3 | B-8 | H-1 |
| C-116 | A.2-4 | B-8 | H-1 |
| C-117 | A.2-5 | B-8 | H-1 |
| C-118 | A.2-6 | B-8 | H-1 |
| C-119 | A.2-7 | B-8 | H-1 |
| C-120 | A.2-8 | B-8 | H-1 |
| C-121 | A.2-9 | B-8 | H-1 |
| C-122 | A.2-10 | B-8 | H-1 |
| C-123 | A.2-11 | B-8 | H-1 |
| C-124 | A.2-12 | B-8 | H-1 |
| C-125 | A.2-13 | B-8 | H-1 |
| C-126 | A.2-14 | B-8 | H-1 |
| C-127 | A.2-15 | B-8 | H-1 |
| C-128 | A.2-16 | B-8 | H-1 |
| C-129 | A.2-1 | B-9 | H-1 |
| C-130 | A.2-2 | B-9 | H-1 |
| C-131 | A.2-3 | B-9 | H-1 |
| C-132 | A.2-4 | B-9 | H-1 |
| C-133 | A.2-5 | B-9 | H-1 |
| C-134 | A.2-6 | B-9 | H-1 |
| C-135 | A.2-7 | B-9 | H-1 |
| C-136 | A.2-8 | B-9 | H-1 |
| C-137 | A.2-9 | B-9 | H-1 |
| C-138 | A.2-10 | B-9 | H-1 |
| C-139 | A.2-11 | B-9 | H-1 |
| C-140 | A.2-12 | B-9 | H-1 |
| C-141 | A.2-13 | B-9 | H-1 |
| C-142 | A.2-14 | B-9 | H-1 |
| C-143 | A.2-15 | B-9 | H-1 |
| C-144 | A.2-16 | B-9 | H-1 |
| C-145 | A.2-1 | B-10 | H-1 |
| C-146 | A.2-2 | B-10 | H-1 |
| C-147 | A.2-3 | B-10 | H-1 |
| C-148 | A.2-4 | B-10 | H-1 |
| C-149 | A.2-5 | B-10 | H-1 |
| C-150 | A.2-6 | B-10 | H-1 |
| C-151 | A.2-7 | B-10 | H-1 |
| C-152 | A.2-8 | B-10 | H-1 |
| C-153 | A.2-9 | B-10 | H-1 |
| C-154 | A.2-10 | B-10 | H-1 |
| C-155 | A.2-11 | B-10 | H-1 |
| C-156 | A.2-12 | B-10 | H-1 |
| C-157 | A.2-13 | B-10 | H-1 |
| C-158 | A.2-14 | B-10 | H-1 |
| C-159 | A.2-15 | B-10 | H-1 |
| C-160 | A.2-16 | B-10 | H-1 |
| C-161 | A.2-1 | B-11 | H-1 |
| C-162 | A.2-2 | B-11 | H-1 |
| C-163 | A.2-3 | B-11 | H-1 |
| C-164 | A.2-4 | B-11 | H-1 |
| C-165 | A.2-5 | B-11 | H-1 |
| C-166 | A.2-6 | B-11 | H-1 |
| C-167 | A.2-7 | B-11 | H-1 |
| C-168 | A.2-8 | B-11 | H-1 |
| C-169 | A.2-9 | B-11 | H-1 |
| C-170 | A.2-10 | B-11 | H-1 |
| C-171 | A.2-11 | B-11 | H-1 |
| C-172 | A.2-12 | B-11 | H-1 |
| C-173 | A.2-13 | B-11 | H-1 |
| C-174 | A.2-14 | B-11 | H-1 |
| C-175 | A.2-15 | B-11 | H-1 |
| C-176 | A.2-16 | B-11 | H-1 |
| C-177 | A.2-1 | B-12 | H-1 |
| C-178 | A.2-2 | B-12 | H-1 |
| C-179 | A.2-3 | B-12 | H-1 |
| C-180 | A.2-4 | B-12 | H-1 |
| C-181 | A.2-5 | B-12 | H-1 |
| C-182 | A.2-6 | B-12 | H-1 |
| C-183 | A.2-7 | B-12 | H-1 |
| C-184 | A.2-8 | B-12 | H-1 |
| C-185 | A.2-9 | B-12 | H-1 |
| C-186 | A.2-10 | B-12 | H-1 |
| C-187 | A.2-11 | B-12 | H-1 |
| C-188 | A.2-12 | B-12 | H-1 |
| C-189 | A.2-13 | B-12 | H-1 |
| C-190 | A.2-14 | B-12 | H-1 |
| C-191 | A.2-15 | B-12 | H-1 |
| C-192 | A.2-16 | B-12 | H-1 |
| C-193 | A.2-1 | B-1 | H-3 |
| C-194 | A.2-2 | B-1 | H-3 |
| C-195 | A.2-3 | B-1 | H-3 |
| C-196 | A.2-4 | B-1 | H-3 |
| C-197 | A.2-5 | B-1 | H-3 |
| C-198 | A.2-6 | B-1 | H-3 |
| C-199 | A.2-7 | B-1 | H-3 |
| C-200 | A.2-8 | B-1 | H-3 |
| C-201 | A.2-9 | B-1 | H-3 |
| C-202 | A.2-10 | B-1 | H-3 |
| C-203 | A.2-11 | B-1 | H-3 |
| C-204 | A.2-12 | B-1 | H-3 |
| C-205 | A.2-13 | B-1 | H-3 |
| C-206 | A.2-14 | B-1 | H-3 |
| C-207 | A.2-15 | B-1 | H-3 |
| C-208 | A.2-16 | B-1 | H-3 |
| C-209 | A.2-1 | B-2 | H-3 |
| C-210 | A.2-2 | B-2 | H-3 |
| C-211 | A.2-3 | B-2 | H-3 |
| C-212 | A.2-4 | B-2 | H-3 |
| C-213 | A.2-5 | B-2 | H-3 |
| C-214 | A.2-6 | B-2 | H-3 |
| C-215 | A.2-7 | B-2 | H-3 |
| C-216 | A.2-8 | B-2 | H-3 |
| C-217 | A.2-9 | B-2 | H-3 |
| C-218 | A.2-10 | B-2 | H-3 |
| C-219 | A.2-11 | B-2 | H-3 |
| C-220 | A.2-12 | B-2 | H-3 |
| C-221 | A.2-13 | B-2 | H-3 |
| C-222 | A.2-14 | B-2 | H-3 |
| C-223 | A.2-15 | B-2 | H-3 |
| C-224 | A.2-16 | B-2 | H-3 |
| C-225 | A.2-1 | B-3 | H-3 |
| C-226 | A.2-2 | B-3 | H-3 |
| C-227 | A.2-3 | B-3 | H-3 |
| C-228 | A.2-4 | B-3 | H-3 |
| C-229 | A.2-5 | B-3 | H-3 |
| C-230 | A.2-6 | B-3 | H-3 |
| C-231 | A.2-7 | B-3 | H-3 |
| C-232 | A.2-8 | B-3 | H-3 |
| C-233 | A.2-9 | B-3 | H-3 |
| C-234 | A.2-10 | B-3 | H-3 |
| C-235 | A.2-11 | B-3 | H-3 |
| C-236 | A.2-12 | B-3 | H-3 |
| C-237 | A.2-13 | B-3 | H-3 |
| C-238 | A.2-14 | B-3 | H-3 |
| C-239 | A.2-15 | B-3 | H-3 |
| C-240 | A.2-16 | B-3 | H-3 |
| C-241 | A.2-1 | B-4 | H-3 |
| C-242 | A.2-2 | B-4 | H-3 |
| C-243 | A.2-3 | B-4 | H-3 |
| C-244 | A.2-4 | B-4 | H-3 |
| C-245 | A.2-5 | B-4 | H-3 |
| C-246 | A.2-6 | B-4 | H-3 |
| C-247 | A.2-7 | B-4 | H-3 |
| C-248 | A.2-8 | B-4 | H-3 |
| C-249 | A.2-9 | B-4 | H-3 |
| C-250 | A.2-10 | B-4 | H-3 |
| C-251 | A.2-11 | B-4 | H-3 |
| C-252 | A.2-12 | B-4 | H-3 |
| C-253 | A.2-13 | B-4 | H-3 |
| C-254 | A.2-14 | B-4 | H-3 |
| C-255 | A.2-15 | B-4 | H-3 |
| C-256 | A.2-16 | B-4 | H-3 |
| C-257 | A.2-1 | B-5 | H-3 |
| C-258 | A.2-2 | B-5 | H-3 |
| C-259 | A.2-3 | B-5 | H-3 |
| C-260 | A.2-4 | B-5 | H-3 |
| C-261 | A.2-5 | B-5 | H-3 |
| C-262 | A.2-6 | B-5 | H-3 |
| C-263 | A.2-7 | B-5 | H-3 |
| C-264 | A.2-8 | B-5 | H-3 |
| C-265 | A.2-9 | B-5 | H-3 |
| C-266 | A.2-10 | B-5 | H-3 |
| C-267 | A.2-11 | B-5 | H-3 |
| C-268 | A.2-12 | B-5 | H-3 |
| C-269 | A.2-13 | B-5 | H-3 |
| C-270 | A.2-14 | B-5 | H-3 |
| C-271 | A.2-15 | B-5 | H-3 |
| C-272 | A.2-16 | B-5 | H-3 |
| C-273 | A.2-1 | B-6 | H-3 |
| C-274 | A.2-2 | B-6 | H-3 |
| C-275 | A.2-3 | B-6 | H-3 |
| C-276 | A.2-4 | B-6 | H-3 |
| C-277 | A.2-5 | B-6 | H-3 |
| C-278 | A.2-6 | B-6 | H-3 |
| C-279 | A.2-7 | B-6 | H-3 |
| C-280 | A.2-8 | B-6 | H-3 |
| C-281 | A.2-9 | B-6 | H-3 |
| C-282 | A.2-10 | B-6 | H-3 |
| C-283 | A.2-11 | B-6 | H-3 |
| C-284 | A.2-12 | B-6 | H-3 |
| C-285 | A.2-13 | B-6 | H-3 |
| C-286 | A.2-14 | B-6 | H-3 |
| C-287 | A.2-15 | B-6 | H-3 |
| C-288 | A.2-16 | B-6 | H-3 |
| C-289 | A.2-1 | B-7 | H-3 |
| C-290 | A.2-2 | B-7 | H-3 |
| C-291 | A.2-3 | B-7 | H-3 |
| C-292 | A.2-4 | B-7 | H-3 |
| C-293 | A.2-5 | B-7 | H-3 |
| C-294 | A.2-6 | B-7 | H-3 |
| C-295 | A.2-7 | B-7 | H-3 |
| C-296 | A.2-8 | B-7 | H-3 |
| C-297 | A.2-9 | B-7 | H-3 |
| C-298 | A.2-10 | B-7 | H-3 |
| C-299 | A.2-11 | B-7 | H-3 |
| C-300 | A.2-12 | B-7 | H-3 |
| C-301 | A.2-13 | B-7 | H-3 |
| C-302 | A.2-14 | B-7 | H-3 |
| C-303 | A.2-15 | B-7 | H-3 |
| C-304 | A.2-16 | B-7 | H-3 |
| C-305 | A.2-1 | B-8 | H-3 |
| C-306 | A.2-2 | B-8 | H-3 |
| C-307 | A.2-3 | B-8 | H-3 |
| C-308 | A.2-4 | B-8 | H-3 |
| C-309 | A.2-5 | B-8 | H-3 |
| C-310 | A.2-6 | B-8 | H-3 |
| C-311 | A.2-7 | B-8 | H-3 |
| C-312 | A.2-8 | B-8 | H-3 |
| C-313 | A.2-9 | B-8 | H-3 |
| C-314 | A.2-10 | B-8 | H-3 |
| C-315 | A.2-11 | B-8 | H-3 |
| C-316 | A.2-12 | B-8 | H-3 |
| C-317 | A.2-13 | B-8 | H-3 |
| C-318 | A.2-14 | B-8 | H-3 |
| C-319 | A.2-15 | B-8 | H-3 |
| C-320 | A.2-16 | B-8 | H-3 |
| C-321 | A.2-1 | B-9 | H-3 |
| C-322 | A.2-2 | B-9 | H-3 |
| C-323 | A.2-3 | B-9 | H-3 |
| C-324 | A.2-4 | B-9 | H-3 |
| C-325 | A.2-5 | B-9 | H-3 |
| C-326 | A.2-6 | B-9 | H-3 |
| C-327 | A.2-7 | B-9 | H-3 |
| C-328 | A.2-8 | B-9 | H-3 |
| C-329 | A.2-9 | B-9 | H-3 |
| C-330 | A.2-10 | B-9 | H-3 |
| C-331 | A.2-11 | B-9 | H-3 |
| C-332 | A.2-12 | B-9 | H-3 |
| C-333 | A.2-13 | B-9 | H-3 |
| C-334 | A.2-14 | B-9 | H-3 |
| C-335 | A.2-15 | B-9 | H-3 |
| C-336 | A.2-16 | B-9 | H-3 |
| C-337 | A.2-1 | B-10 | H-3 |
| C-338 | A.2-2 | B-10 | H-3 |
| C-339 | A.2-3 | B-10 | H-3 |
| C-340 | A.2-4 | B-10 | H-3 |
| C-341 | A.2-5 | B-10 | H-3 |
| C-342 | A.2-6 | B-10 | H-3 |
| C-343 | A.2-7 | B-10 | H-3 |
| C-344 | A.2-8 | B-10 | H-3 |
| C-345 | A.2-9 | B-10 | H-3 |
| C-346 | A.2-10 | B-10 | H-3 |
| C-347 | A.2-11 | B-10 | H-3 |
| C-348 | A.2-12 | B-10 | H-3 |
| C-349 | A.2-13 | B-10 | H-3 |
| C-350 | A.2-14 | B-10 | H-3 |
| C-351 | A.2-15 | B-10 | H-3 |
| C-352 | A.2-16 | B-10 | H-3 |
| C-353 | A.2-1 | B-11 | H-3 |
| C-354 | A.2-2 | B-11 | H-3 |
| C-355 | A.2-3 | B-11 | H-3 |
| C-356 | A.2-4 | B-11 | H-3 |
| C-357 | A.2-5 | B-11 | H-3 |
| C-358 | A.2-6 | B-11 | H-3 |
| C-359 | A.2-7 | B-11 | H-3 |
| C-360 | A.2-8 | B-11 | H-3 |
| C-361 | A.2-9 | B-11 | H-3 |
| C-362 | A.2-10 | B-11 | H-3 |
| C-363 | A.2-11 | B-11 | H-3 |
| C-364 | A.2-12 | B-11 | H-3 |
| C-365 | A.2-13 | B-11 | H-3 |
| C-366 | A.2-14 | B-11 | H-3 |
| C-367 | A.2-15 | B-11 | H-3 |
| C-368 | A.2-16 | B-11 | H-3 |
| C-369 | A.2-1 | B-12 | H-3 |
| C-370 | A.2-2 | B-12 | H-3 |
| C-371 | A.2-3 | B-12 | H-3 |
| C-372 | A.2-4 | B-12 | H-3 |
| C-373 | A.2-5 | B-12 | H-3 |
| C-374 | A.2-6 | B-12 | H-3 |
| C-375 | A.2-7 | B-12 | H-3 |
| C-376 | A.2-8 | B-12 | H-3 |
| C-377 | A.2-9 | B-12 | H-3 |
| C-378 | A.2-10 | B-12 | H-3 |
| C-379 | A.2-11 | B-12 | H-3 |
| C-380 | A.2-12 | B-12 | H-3 |
| C-381 | A.2-13 | B-12 | H-3 |
| C-382 | A.2-14 | B-12 | H-3 |
| C-383 | A.2-15 | B-12 | H-3 |
| C-384 | A.2-16 | B-12 | H-3 |
| C-385 | A.2-1 | B-1 | H-14 |
| C-386 | A.2-2 | B-1 | H-14 |
| C-387 | A.2-3 | B-1 | H-14 |
| C-388 | A.2-4 | B-1 | H-14 |
| C-389 | A.2-5 | B-1 | H-14 |
| C-390 | A.2-6 | B-1 | H-14 |
| C-391 | A.2-7 | B-1 | H-14 |
| C-392 | A.2-8 | B-1 | H-14 |
| C-393 | A.2-9 | B-1 | H-14 |
| C-394 | A.2-10 | B-1 | H-14 |
| C-395 | A.2-11 | B-1 | H-14 |
| C-396 | A.2-12 | B-1 | H-14 |
| C-397 | A.2-13 | B-1 | H-14 |
| C-398 | A.2-14 | B-1 | H-14 |
| C-399 | A.2-15 | B-1 | H-14 |
| C-400 | A.2-16 | B-1 | H-14 |
| C-401 | A.2-1 | B-2 | H-14 |
| C-402 | A.2-2 | B-2 | H-14 |
| C-403 | A.2-3 | B-2 | H-14 |
| C-404 | A.2-4 | B-2 | H-14 |
| C-405 | A.2-5 | B-2 | H-14 |
| C-406 | A.2-6 | B-2 | H-14 |
| C-407 | A.2-7 | B-2 | H-14 |
| C-408 | A.2-8 | B-2 | H-14 |
| C-409 | A.2-9 | B-2 | H-14 |
| C-410 | A.2-10 | B-2 | H-14 |
| C-411 | A.2-11 | B-2 | H-14 |
| C-412 | A.2-12 | B-2 | H-14 |
| C-413 | A.2-13 | B-2 | H-14 |
| C-414 | A.2-14 | B-2 | H-14 |
| C-415 | A.2-15 | B-2 | H-14 |
| C-416 | A.2-16 | B-2 | H-14 |
| C-417 | A.2-1 | B-3 | H-14 |
| C-418 | A.2-2 | B-3 | H-14 |
| C-419 | A.2-3 | B-3 | H-14 |
| C-420 | A.2-4 | B-3 | H-14 |
| C-421 | A.2-5 | B-3 | H-14 |
| C-422 | A.2-6 | B-3 | H-14 |
| C-423 | A.2-7 | B-3 | H-14 |
| C-424 | A.2-8 | B-3 | H-14 |
| C-425 | A.2-9 | B-3 | H-14 |
| C-426 | A.2-10 | B-3 | H-14 |
| C-427 | A.2-11 | B-3 | H-14 |
| C-428 | A.2-12 | B-3 | H-14 |
| C-429 | A.2-13 | B-3 | H-14 |
| C-430 | A.2-14 | B-3 | H-14 |
| C-431 | A.2-15 | B-3 | H-14 |
| C-432 | A.2-16 | B-3 | H-14 |
| C-433 | A.2-1 | B-4 | H-14 |
| C-434 | A.2-2 | B-4 | H-14 |
| C-435 | A.2-3 | B-4 | H-14 |
| C-436 | A.2-4 | B-4 | H-14 |
| C-437 | A.2-5 | B-4 | H-14 |
| C-438 | A.2-6 | B-4 | H-14 |
| C-439 | A.2-7 | B-4 | H-14 |
| C-440 | A.2-8 | B-4 | H-14 |
| C-441 | A.2-9 | B-4 | H-14 |
| C-442 | A.2-10 | B-4 | H-14 |
| C-443 | A.2-11 | B-4 | H-14 |
| C-444 | A.2-12 | B-4 | H-14 |
| C-445 | A.2-13 | B-4 | H-14 |
| C-446 | A.2-14 | B-4 | H-14 |
| C-447 | A.2-15 | B-4 | H-14 |
| C-448 | A.2-16 | B-4 | H-14 |
| C-449 | A.2-1 | B-5 | H-14 |
| C-450 | A.2-2 | B-5 | H-14 |
| C-451 | A.2-3 | B-5 | H-14 |
| C-452 | A.2-4 | B-5 | H-14 |
| C-453 | A.2-5 | B-5 | H-14 |
| C-454 | A.2-6 | B-5 | H-14 |
| C-455 | A.2-7 | B-5 | H-14 |
| C-456 | A.2-8 | B-5 | H-14 |
| C-457 | A.2-9 | B-5 | H-14 |
| C-458 | A.2-10 | B-5 | H-14 |
| C-459 | A.2-11 | B-5 | H-14 |
| C-460 | A.2-12 | B-5 | H-14 |
| C-461 | A.2-13 | B-5 | H-14 |
| C-462 | A.2-14 | B-5 | H-14 |
| C-463 | A.2-15 | B-5 | H-14 |
| C-464 | A.2-16 | B-5 | H-14 |
| C-465 | A.2-1 | B-6 | H-14 |
| C-466 | A.2-2 | B-6 | H-14 |
| C-467 | A.2-3 | B-6 | H-14 |
| C-468 | A.2-4 | B-6 | H-14 |
| C-469 | A.2-5 | B-6 | H-14 |
| C-470 | A.2-6 | B-6 | H-14 |
| C-471 | A.2-7 | B-6 | H-14 |
| C-472 | A.2-8 | B-6 | H-14 |
| C-473 | A.2-9 | B-6 | H-14 |
| C-474 | A.2-10 | B-6 | H-14 |
| C-475 | A.2-11 | B-6 | H-14 |
| C-476 | A.2-12 | B-6 | H-14 |
| C-477 | A.2-13 | B-6 | H-14 |
| C-478 | A.2-14 | B-6 | H-14 |
| C-479 | A.2-15 | B-6 | H-14 |
| C-480 | A.2-16 | B-6 | H-14 |
| C-481 | A.2-1 | B-7 | H-14 |
| C-482 | A.2-2 | B-7 | H-14 |
| C-483 | A.2-3 | B-7 | H-14 |
| C-484 | A.2-4 | B-7 | H-14 |
| C-485 | A.2-5 | B-7 | H-14 |
| C-486 | A.2-6 | B-7 | H-14 |
| C-487 | A.2-7 | B-7 | H-14 |
| C-488 | A.2-8 | B-7 | H-14 |
| C-489 | A.2-9 | B-7 | H-14 |
| C-490 | A.2-10 | B-7 | H-14 |
| C-491 | A.2-11 | B-7 | H-14 |
| C-492 | A.2-12 | B-7 | H-14 |
| C-493 | A.2-13 | B-7 | H-14 |
| C-494 | A.2-14 | B-7 | H-14 |
| C-495 | A.2-15 | B-7 | H-14 |
| C-496 | A.2-16 | B-7 | H-14 |
| C-497 | A.2-1 | B-8 | H-14 |
| C-498 | A.2-2 | B-8 | H-14 |
| C-499 | A.2-3 | B-8 | H-14 |
| C-500 | A.2-4 | B-8 | H-14 |
| C-501 | A.2-5 | B-8 | H-14 |
| C-502 | A.2-6 | B-8 | H-14 |
| C-503 | A.2-7 | B-8 | H-14 |
| C-504 | A.2-8 | B-8 | H-14 |
| C-505 | A.2-9 | B-8 | H-14 |
| C-506 | A.2-10 | B-8 | H-14 |
| C-507 | A.2-11 | B-8 | H-14 |
| C-508 | A.2-12 | B-8 | H-14 |
| C-509 | A.2-13 | B-8 | H-14 |
| C-510 | A.2-14 | B-8 | H-14 |
| C-511 | A.2-15 | B-8 | H-14 |
| C-512 | A.2-16 | B-8 | H-14 |
| C-513 | A.2-1 | B-9 | H-14 |
| C-514 | A.2-2 | B-9 | H-14 |
| C-515 | A.2-3 | B-9 | H-14 |
| C-516 | A.2-4 | B-9 | H-14 |
| C-517 | A.2-5 | B-9 | H-14 |
| C-518 | A.2-6 | B-9 | H-14 |
| C-519 | A.2-7 | B-9 | H-14 |
| C-520 | A.2-8 | B-9 | H-14 |
| C-521 | A.2-9 | B-9 | H-14 |
| C-522 | A.2-10 | B-9 | H-14 |
| C-523 | A.2-11 | B-9 | H-14 |
| C-524 | A.2-12 | B-9 | H-14 |
| C-525 | A.2-13 | B-9 | H-14 |
| C-526 | A.2-14 | B-9 | H-14 |
| C-527 | A.2-15 | B-9 | H-14 |
| C-528 | A.2-16 | B-9 | H-14 |
| C-529 | A.2-1 | B-10 | H-14 |
| C-530 | A.2-2 | B-10 | H-14 |
| C-531 | A.2-3 | B-10 | H-14 |
| C-532 | A.2-4 | B-10 | H-14 |
| C-533 | A.2-5 | B-10 | H-14 |
| C-534 | A.2-6 | B-10 | H-14 |
| C-535 | A.2-7 | B-10 | H-14 |
| C-536 | A.2-8 | B-10 | H-14 |
| C-537 | A.2-9 | B-10 | H-14 |
| C-538 | A.2-10 | B-10 | H-14 |
| C-539 | A.2-11 | B-10 | H-14 |
| C-540 | A.2-12 | B-10 | H-14 |
| C-541 | A.2-13 | B-10 | H-14 |
| C-542 | A.2-14 | B-10 | H-14 |
| C-543 | A.2-15 | B-10 | H-14 |
| C-544 | A.2-16 | B-10 | H-14 |
| C-545 | A.2-1 | B-11 | H-14 |
| C-546 | A.2-2 | B-11 | H-14 |
| C-547 | A.2-3 | B-11 | H-14 |
| C-548 | A.2-4 | B-11 | H-14 |
| C-549 | A.2-5 | B-11 | H-14 |
| C-550 | A.2-6 | B-11 | H-14 |
| C-551 | A.2-7 | B-11 | H-14 |
| C-552 | A.2-8 | B-11 | H-14 |
| C-553 | A.2-9 | B-11 | H-14 |
| C-554 | A.2-10 | B-11 | H-14 |
| C-555 | A.2-11 | B-11 | H-14 |
| C-556 | A.2-12 | B-11 | H-14 |
| C-557 | A.2-13 | B-11 | H-14 |
| C-558 | A.2-14 | B-11 | H-14 |
| C-559 | A.2-15 | B-11 | H-14 |
| C-560 | A.2-16 | B-11 | H-14 |
| C-561 | A.2-1 | B-12 | H-14 |
| C-562 | A.2-2 | B-12 | H-14 |
| C-563 | A.2-3 | B-12 | H-14 |
| C-564 | A.2-4 | B-12 | H-14 |
| C-565 | A.2-5 | B-12 | H-14 |
| C-566 | A.2-6 | B-12 | H-14 |
| C-567 | A.2-7 | B-12 | H-14 |
| C-568 | A.2-8 | B-12 | H-14 |
| C-569 | A.2-9 | B-12 | H-14 |
| C-570 | A.2-10 | B-12 | H-14 |
| C-571 | A.2-11 | B-12 | H-14 |
| C-572 | A.2-12 | B-12 | H-14 |
| C-573 | A.2-13 | B-12 | H-14 |
| C-574 | A.2-14 | B-12 | H-14 |
| C-575 | A.2-15 | B-12 | H-14 |
| C-576 | A.2-16 | B-12 | H-14 |
| C-577 | A.2-1 | B-1 | H-16 |
| C-578 | A.2-2 | B-1 | H-16 |
| C-579 | A.2-3 | B-1 | H-16 |
| C-580 | A.2-4 | B-1 | H-16 |
| C-581 | A.2-5 | B-1 | H-16 |
| C-582 | A.2-6 | B-1 | H-16 |
| C-583 | A.2-7 | B-1 | H-16 |
| C-584 | A.2-8 | B-1 | H-16 |
| C-585 | A.2-9 | B-1 | H-16 |
| C-586 | A.2-10 | B-1 | H-16 |
| C-587 | A.2-11 | B-1 | H-16 |
| C-588 | A.2-12 | B-1 | H-16 |
| C-589 | A.2-13 | B-1 | H-16 |
| C-590 | A.2-14 | B-1 | H-16 |
| C-591 | A.2-15 | B-1 | H-16 |
| C-592 | A.2-16 | B-1 | H-16 |
| C-593 | A.2-1 | B-2 | H-16 |
| C-594 | A.2-2 | B-2 | H-16 |
| C-595 | A.2-3 | B-2 | H-16 |
| C-596 | A.2-4 | B-2 | H-16 |
| C-597 | A.2-5 | B-2 | H-16 |
| C-598 | A.2-6 | B-2 | H-16 |
| C-599 | A.2-7 | B-2 | H-16 |
| C-600 | A.2-8 | B-2 | H-16 |
| C-601 | A.2-9 | B-2 | H-16 |
| C-602 | A.2-10 | B-2 | H-16 |
| C-603 | A.2-11 | B-2 | H-16 |
| C-604 | A.2-12 | B-2 | H-16 |
| C-605 | A.2-13 | B-2 | H-16 |
| C-606 | A.2-14 | B-2 | H-16 |
| C-607 | A.2-15 | B-2 | H-16 |
| C-608 | A.2-16 | B-2 | H-16 |
| C-609 | A.2-1 | B-3 | H-16 |
| C-610 | A.2-2 | B-3 | H-16 |
| C-611 | A.2-3 | B-3 | H-16 |
| C-612 | A.2-4 | B-3 | H-16 |
| C-613 | A.2-5 | B-3 | H-16 |
| C-614 | A.2-6 | B-3 | H-16 |
| C-615 | A.2-7 | B-3 | H-16 |
| C-616 | A.2-8 | B-3 | H-16 |
| C-617 | A.2-9 | B-3 | H-16 |
| C-618 | A.2-10 | B-3 | H-16 |
| C-619 | A.2-11 | B-3 | H-16 |
| C-620 | A.2-12 | B-3 | H-16 |
| C-621 | A.2-13 | B-3 | H-16 |
| C-622 | A.2-14 | B-3 | H-16 |
| C-623 | A.2-15 | B-3 | H-16 |
| C-624 | A.2-16 | B-3 | H-16 |
| C-625 | A.2-1 | B-4 | H-16 |
| C-626 | A.2-2 | B-4 | H-16 |
| C-627 | A.2-3 | B-4 | H-16 |
| C-628 | A.2-4 | B-4 | H-16 |
| C-629 | A.2-5 | B-4 | H-16 |
| C-630 | A.2-6 | B-4 | H-16 |
| C-631 | A.2-7 | B-4 | H-16 |
| C-632 | A.2-8 | B-4 | H-16 |
| C-633 | A.2-9 | B-4 | H-16 |
| C-634 | A.2-10 | B-4 | H-16 |
| C-635 | A.2-11 | B-4 | H-16 |
| C-636 | A.2-12 | B-4 | H-16 |
| C-637 | A.2-13 | B-4 | H-16 |
| C-638 | A.2-14 | B-4 | H-16 |
| C-639 | A.2-15 | B-4 | H-16 |
| C-640 | A.2-16 | B-4 | H-16 |
| C-641 | A.2-1 | B-5 | H-16 |
| C-642 | A.2-2 | B-5 | H-16 |
| C-643 | A.2-3 | B-5 | H-16 |
| C-644 | A.2-4 | B-5 | H-16 |
| C-645 | A.2-5 | B-5 | H-16 |
| C-646 | A.2-6 | B-5 | H-16 |
| C-647 | A.2-7 | B-5 | H-16 |
| C-648 | A.2-8 | B-5 | H-16 |
| C-649 | A.2-9 | B-5 | H-16 |
| C-650 | A.2-10 | B-5 | H-16 |
| C-651 | A.2-11 | B-5 | H-16 |
| C-652 | A.2-12 | B-5 | H-16 |
| C-653 | A.2-13 | B-5 | H-16 |
| C-654 | A.2-14 | B-5 | H-16 |
| C-655 | A.2-15 | B-5 | H-16 |
| C-656 | A.2-16 | B-5 | H-16 |
| C-657 | A.2-1 | B-6 | H-16 |
| C-658 | A.2-2 | B-6 | H-16 |
| C-659 | A.2-3 | B-6 | H-16 |
| C-660 | A.2-4 | B-6 | H-16 |
| C-661 | A.2-5 | B-6 | H-16 |
| C-662 | A.2-6 | B-6 | H-16 |
| C-663 | A.2-7 | B-6 | H-16 |
| C-664 | A.2-8 | B-6 | H-16 |
| C-665 | A.2-9 | B-6 | H-16 |
| C-666 | A.2-10 | B-6 | H-16 |
| C-667 | A.2-11 | B-6 | H-16 |
| C-668 | A.2-12 | B-6 | H-16 |
| C-669 | A.2-13 | B-6 | H-16 |
| C-670 | A.2-14 | B-6 | H-16 |
| C-671 | A.2-15 | B-6 | H-16 |
| C-672 | A.2-16 | B-6 | H-16 |
| C-673 | A.2-1 | B-7 | H-16 |
| C-674 | A.2-2 | B-7 | H-16 |
| C-675 | A.2-3 | B-7 | H-16 |
| C-676 | A.2-4 | B-7 | H-16 |
| C-677 | A.2-5 | B-7 | H-16 |
| C-678 | A.2-6 | B-7 | H-16 |
| C-679 | A.2-7 | B-7 | H-16 |
| C-680 | A.2-8 | B-7 | H-16 |
| C-681 | A.2-9 | B-7 | H-16 |
| C-682 | A.2-10 | B-7 | H-16 |
| C-683 | A.2-11 | B-7 | H-16 |
| C-684 | A.2-12 | B-7 | H-16 |
| C-685 | A.2-13 | B-7 | H-16 |
| C-686 | A.2-14 | B-7 | H-16 |
| C-687 | A.2-15 | B-7 | H-16 |
| C-688 | A.2-16 | B-7 | H-16 |
| C-689 | A.2-1 | B-8 | H-16 |
| C-690 | A.2-2 | B-8 | H-16 |
| C-691 | A.2-3 | B-8 | H-16 |
| C-692 | A.2-4 | B-8 | H-16 |
| C-693 | A.2-5 | B-8 | H-16 |
| C-694 | A.2-6 | B-8 | H-16 |
| C-695 | A.2-7 | B-8 | H-16 |
| C-696 | A.2-8 | B-8 | H-16 |
| C-697 | A.2-9 | B-8 | H-16 |
| C-698 | A.2-10 | B-8 | H-16 |
| C-699 | A.2-11 | B-8 | H-16 |
| C-700 | A.2-12 | B-8 | H-16 |
| C-701 | A.2-13 | B-8 | H-16 |
| C-702 | A.2-14 | B-8 | H-16 |
| C-703 | A.2-15 | B-8 | H-16 |
| C-704 | A.2-16 | B-8 | H-16 |
| C-705 | A.2-1 | B-9 | H-16 |
| C-706 | A.2-2 | B-9 | H-16 |
| C-707 | A.2-3 | B-9 | H-16 |
| C-708 | A.2-4 | B-9 | H-16 |
| C-709 | A.2-5 | B-9 | H-16 |
| C-710 | A.2-6 | B-9 | H-16 |
| C-711 | A.2-7 | B-9 | H-16 |
| C-712 | A.2-8 | B-9 | H-16 |
| C-713 | A.2-9 | B-9 | H-16 |
| C-714 | A.2-10 | B-9 | H-16 |
| C-715 | A.2-11 | B-9 | H-16 |
| C-716 | A.2-12 | B-9 | H-16 |
| C-717 | A.2-13 | B-9 | H-16 |
| C-718 | A.2-14 | B-9 | H-16 |
| C-719 | A.2-15 | B-9 | H-16 |
| C-720 | A.2-16 | B-9 | H-16 |
| C-721 | A.2-1 | B-10 | H-16 |
| C-722 | A.2-2 | B-10 | H-16 |
| C-723 | A.2-3 | B-10 | H-16 |
| C-724 | A.2-4 | B-10 | H-16 |
| C-725 | A.2-5 | B-10 | H-16 |
| C-726 | A.2-6 | B-10 | H-16 |
| C-727 | A.2-7 | B-10 | H-16 |
| C-728 | A.2-8 | B-10 | H-16 |
| C-729 | A.2-9 | B-10 | H-16 |
| C-730 | A.2-10 | B-10 | H-16 |
| C-731 | A.2-11 | B-10 | H-16 |
| C-732 | A.2-12 | B-10 | H-16 |
| C-733 | A.2-13 | B-10 | H-16 |
| C-734 | A.2-14 | B-10 | H-16 |
| C-735 | A.2-15 | B-10 | H-16 |
| C-736 | A.2-16 | B-10 | H-16 |
| C-737 | A.2-1 | B-11 | H-16 |
| C-738 | A.2-2 | B-11 | H-16 |
| C-739 | A.2-3 | B-11 | H-16 |
| C-740 | A.2-4 | B-11 | H-16 |
| C-741 | A.2-5 | B-11 | H-16 |
| C-742 | A.2-6 | B-11 | H-16 |
| C-743 | A.2-7 | B-11 | H-16 |
| C-744 | A.2-8 | B-11 | H-16 |
| C-745 | A.2-9 | B-11 | H-16 |
| C-746 | A.2-10 | B-11 | H-16 |
| C-747 | A.2-11 | B-11 | H-16 |
| C-748 | A.2-12 | B-11 | H-16 |
| C-749 | A.2-13 | B-11 | H-16 |
| C-750 | A.2-14 | B-11 | H-16 |
| C-751 | A.2-15 | B-11 | H-16 |
| C-752 | A.2-16 | B-11 | H-16 |
| C-753 | A.2-1 | B-12 | H-16 |
| C-754 | A.2-2 | B-12 | H-16 |
| C-755 | A.2-3 | B-12 | H-16 |
| C-756 | A.2-4 | B-12 | H-16 |
| C-757 | A.2-5 | B-12 | H-16 |
| C-758 | A.2-6 | B-12 | H-16 |
| C-759 | A.2-7 | B-12 | H-16 |
| C-760 | A.2-8 | B-12 | H-16 |
| C-761 | A.2-9 | B-12 | H-16 |
| C-762 | A.2-10 | B-12 | H-16 |
| C-763 | A.2-11 | B-12 | H-16 |
| C-764 | A.2-12 | B-12 | H-16 |
| C-765 | A.2-13 | B-12 | H-16 |
| C-766 | A.2-14 | B-12 | H-16 |
| C-767 | A.2-15 | B-12 | H-16 |
| C-768 | A.2-16 | B-12 | H-16 |
| C-769 | A.2-1 | B-1 | H-23 |
| C-770 | A.2-2 | B-1 | H-23 |
| C-771 | A.2-3 | B-1 | H-23 |
| C-772 | A.2-4 | B-1 | H-23 |
| C-773 | A.2-5 | B-1 | H-23 |
| C-774 | A.2-6 | B-1 | H-23 |
| C-775 | A.2-7 | B-1 | H-23 |
| C-776 | A.2-8 | B-1 | H-23 |
| C-777 | A.2-9 | B-1 | H-23 |
| C-778 | A.2-10 | B-1 | H-23 |
| C-779 | A.2-11 | B-1 | H-23 |
| C-780 | A.2-12 | B-1 | H-23 |
| C-781 | A.2-13 | B-1 | H-23 |
| C-782 | A.2-14 | B-1 | H-23 |
| C-783 | A.2-15 | B-1 | H-23 |
| C-784 | A.2-16 | B-1 | H-23 |
| C-785 | A.2-1 | B-2 | H-23 |
| C-786 | A.2-2 | B-2 | H-23 |
| C-787 | A.2-3 | B-2 | H-23 |
| C-788 | A.2-4 | B-2 | H-23 |
| C-789 | A.2-5 | B-2 | H-23 |
| C-790 | A.2-6 | B-2 | H-23 |
| C-791 | A.2-7 | B-2 | H-23 |
| C-792 | A.2-8 | B-2 | H-23 |
| C-793 | A.2-9 | B-2 | H-23 |
| C-794 | A.2-10 | B-2 | H-23 |
| C-795 | A.2-11 | B-2 | H-23 |
| C-796 | A.2-12 | B-2 | H-23 |
| C-797 | A.2-13 | B-2 | H-23 |
| C-798 | A.2-14 | B-2 | H-23 |
| C-799 | A.2-15 | B-2 | H-23 |
| C-800 | A.2-16 | B-2 | H-23 |
| C-801 | A.2-1 | B-3 | H-23 |
| C-802 | A.2-2 | B-3 | H-23 |
| C-803 | A.2-3 | B-3 | H-23 |
| C-804 | A.2-4 | B-3 | H-23 |
| C-805 | A.2-5 | B-3 | H-23 |
| C-806 | A.2-6 | B-3 | H-23 |
| C-807 | A.2-7 | B-3 | H-23 |
| C-808 | A.2-8 | B-3 | H-23 |
| C-809 | A.2-9 | B-3 | H-23 |
| C-810 | A.2-10 | B-3 | H-23 |
| C-811 | A.2-11 | B-3 | H-23 |
| C-812 | A.2-12 | B-3 | H-23 |
| C-813 | A.2-13 | B-3 | H-23 |
| C-814 | A.2-14 | B-3 | H-23 |
| C-815 | A.2-15 | B-3 | H-23 |
| C-816 | A.2-16 | B-3 | H-23 |
| C-817 | A.2-1 | B-4 | H-23 |
| C-818 | A.2-2 | B-4 | H-23 |
| C-819 | A.2-3 | B-4 | H-23 |
| C-820 | A.2-4 | B-4 | H-23 |
| C-821 | A.2-5 | B-4 | H-23 |
| C-822 | A.2-6 | B-4 | H-23 |
| C-823 | A.2-7 | B-4 | H-23 |
| C-824 | A.2-8 | B-4 | H-23 |
| C-825 | A.2-9 | B-4 | H-23 |
| C-826 | A.2-10 | B-4 | H-23 |
| C-827 | A.2-11 | B-4 | H-23 |
| C-828 | A.2-12 | B-4 | H-23 |
| C-829 | A.2-13 | B-4 | H-23 |
| C-830 | A.2-14 | B-4 | H-23 |
| C-831 | A.2-15 | B-4 | H-23 |
| C-832 | A.2-16 | B-4 | H-23 |
| C-833 | A.2-1 | B-5 | H-23 |
| C-834 | A.2-2 | B-5 | H-23 |
| C-835 | A.2-3 | B-5 | H-23 |
| C-836 | A.2-4 | B-5 | H-23 |
| C-837 | A.2-5 | B-5 | H-23 |
| C-838 | A.2-6 | B-5 | H-23 |
| C-839 | A.2-7 | B-5 | H-23 |
| C-840 | A.2-8 | B-5 | H-23 |
| C-841 | A.2-9 | B-5 | H-23 |
| C-842 | A.2-10 | B-5 | H-23 |
| C-843 | A.2-11 | B-5 | H-23 |
| C-844 | A.2-12 | B-5 | H-23 |
| C-845 | A.2-13 | B-5 | H-23 |
| C-846 | A.2-14 | B-5 | H-23 |
| C-847 | A.2-15 | B-5 | H-23 |
| C-848 | A.2-16 | B-5 | H-23 |
| C-849 | A.2-1 | B-6 | H-23 |
| C-850 | A.2-2 | B-6 | H-23 |
| C-851 | A.2-3 | B-6 | H-23 |
| C-852 | A.2-4 | B-6 | H-23 |
| C-853 | A.2-5 | B-6 | H-23 |
| C-854 | A.2-6 | B-6 | H-23 |
| C-855 | A.2-7 | B-6 | H-23 |
| C-856 | A.2-8 | B-6 | H-23 |
| C-857 | A.2-9 | B-6 | H-23 |
| C-858 | A.2-10 | B-6 | H-23 |
| C-859 | A.2-11 | B-6 | H-23 |
| C-860 | A.2-12 | B-6 | H-23 |
| C-861 | A.2-13 | B-6 | H-23 |
| C-862 | A.2-14 | B-6 | H-23 |
| C-863 | A.2-15 | B-6 | H-23 |
| C-864 | A.2-16 | B-6 | H-23 |
| C-865 | A.2-1 | B-7 | H-23 |
| C-866 | A.2-2 | B-7 | H-23 |
| C-867 | A.2-3 | B-7 | H-23 |
| C-868 | A.2-4 | B-7 | H-23 |
| C-869 | A.2-5 | B-7 | H-23 |
| C-870 | A.2-6 | B-7 | H-23 |
| C-871 | A.2-7 | B-7 | H-23 |
| C-872 | A.2-8 | B-7 | H-23 |
| C-873 | A.2-9 | B-7 | H-23 |
| C-874 | A.2-10 | B-7 | H-23 |
| C-875 | A.2-11 | B-7 | H-23 |
| C-876 | A.2-12 | B-7 | H-23 |
| C-877 | A.2-13 | B-7 | H-23 |
| C-878 | A.2-14 | B-7 | H-23 |
| C-879 | A.2-15 | B-7 | H-23 |
| C-880 | A.2-16 | B-7 | H-23 |
| C-881 | A.2-1 | B-8 | H-23 |
| C-882 | A.2-2 | B-8 | H-23 |
| C-883 | A.2-3 | B-8 | H-23 |
| C-884 | A.2-4 | B-8 | H-23 |
| C-885 | A.2-5 | B-8 | H-23 |
| C-886 | A.2-6 | B-8 | H-23 |
| C-887 | A.2-7 | B-8 | H-23 |
| C-888 | A.2-8 | B-8 | H-23 |
| C-889 | A.2-9 | B-8 | H-23 |
| C-890 | A.2-10 | B-8 | H-23 |
| C-891 | A.2-11 | B-8 | H-23 |
| C-892 | A.2-12 | B-8 | H-23 |
| C-893 | A.2-13 | B-8 | H-23 |
| C-894 | A.2-14 | B-8 | H-23 |
| C-895 | A.2-15 | B-8 | H-23 |
| C-896 | A.2-16 | B-8 | H-23 |
| C-897 | A.2-1 | B-9 | H-23 |
| C-898 | A.2-2 | B-9 | H-23 |
| C-899 | A.2-3 | B-9 | H-23 |
| C-900 | A.2-4 | B-9 | H-23 |
| C-901 | A.2-5 | B-9 | H-23 |
| C-902 | A.2-6 | B-9 | H-23 |
| C-903 | A.2-7 | B-9 | H-23 |
| C-904 | A.2-8 | B-9 | H-23 |
| C-905 | A.2-9 | B-9 | H-23 |
| C-906 | A.2-10 | B-9 | H-23 |
| C-907 | A.2-11 | B-9 | H-23 |
| C-908 | A.2-12 | B-9 | H-23 |
| C-909 | A.2-13 | B-9 | H-23 |
| C-910 | A.2-14 | B-9 | H-23 |
| C-911 | A.2-15 | B-9 | H-23 |
| C-912 | A.2-16 | B-9 | H-23 |
| C-913 | A.2-1 | B-10 | H-23 |
| C-914 | A.2-2 | B-10 | H-23 |
| C-915 | A.2-3 | B-10 | H-23 |
| C-916 | A.2-4 | B-10 | H-23 |
| C-917 | A.2-5 | B-10 | H-23 |
| C-918 | A.2-6 | B-10 | H-23 |
| C-919 | A.2-7 | B-10 | H-23 |
| C-920 | A.2-8 | B-10 | H-23 |
| C-921 | A.2-9 | B-10 | H-23 |
| C-922 | A.2-10 | B-10 | H-23 |
| C-923 | A.2-11 | B-10 | H-23 |
| C-924 | A.2-12 | B-10 | H-23 |
| C-925 | A.2-13 | B-10 | H-23 |
| C-926 | A.2-14 | B-10 | H-23 |
| C-927 | A.2-15 | B-10 | H-23 |
| C-928 | A.2-16 | B-10 | H-23 |
| C-929 | A.2-1 | B-11 | H-23 |
| C-930 | A.2-2 | B-11 | H-23 |
| C-931 | A.2-3 | B-11 | H-23 |
| C-932 | A.2-4 | B-11 | H-23 |
| C-933 | A.2-5 | B-11 | H-23 |
| C-934 | A.2-6 | B-11 | H-23 |
| C-935 | A.2-7 | B-11 | H-23 |
| C-936 | A.2-8 | B-11 | H-23 |
| C-937 | A.2-9 | B-11 | H-23 |
| C-938 | A.2-10 | B-11 | H-23 |
| C-939 | A.2-11 | B-11 | H-23 |
| C-940 | A.2-12 | B-11 | H-23 |
| C-941 | A.2-13 | B-11 | H-23 |
| C-942 | A.2-14 | B-11 | H-23 |
| C-943 | A.2-15 | B-11 | H-23 |
| C-944 | A.2-16 | B-11 | H-23 |
| C-945 | A.2-1 | B-12 | H-23 |
| C-946 | A.2-2 | B-12 | H-23 |
| C-947 | A.2-3 | B-12 | H-23 |
| C-948 | A.2-4 | B-12 | H-23 |
| C-949 | A.2-5 | B-12 | H-23 |
| C-950 | A.2-6 | B-12 | H-23 |
| C-951 | A.2-7 | B-12 | H-23 |
| C-952 | A.2-8 | B-12 | H-23 |
| C-953 | A.2-9 | B-12 | H-23 |
| C-954 | A.2-10 | B-12 | H-23 |
| C-955 | A.2-11 | B-12 | H-23 |
| C-956 | A.2-12 | B-12 | H-23 |
| C-957 | A.2-13 | B-12 | H-23 |
| C-958 | A.2-14 | B-12 | H-23 |
| C-959 | A.2-15 | B-12 | H-23 |
| C-960 | A.2-16 | B-12 | H-23 |

With respect to their use, preference is also given to the compounds I compiled in tables 1 to 33 below, wherein R, R¹ and R² in each case are hydrogen and the meaning of R^{a5}, R^{a6}, R³, R⁴, n and R^{b} in each case are one of the combinations selected from lines C-1 to C-192 in table C and wherein the meaning of Het is selected from H-2 to H-38 as described in table H. Herein, for example, compound I.192.23 derives its name from the combination of the radicals R^{a5}, R^{a6} with the radicals R³, R⁴, n and R^{b} as defined in line C-192 in table C and the meaning of Het being H-23 as defined in table H, which compound is denominated in line C-960 in table C).

Table 1: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-2 as described in table H (compounds I.1.2 to compound I.192.2).

Table 2: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-4 as described in table H (compounds I.1.4 to compound I.192.4).

Table 3: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-5 as described in table H (compounds I.1.5 to compound I.192.5).

Table 4: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-6 as described in table H (compounds I.1.6 to compound I.192.6).

Table 5: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-7 as described in table H (compounds I.1.7 to compound I.192.7).

Table 6: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-8 as described in table H (compounds I.1.8 to compound I.192.8).

Table 7: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-9 as described in table H (compounds I.1.9 to compound I.192.9).

Table 8: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-10 as described in table H (compounds I.1.10 to compound I.192.10).

Table 9: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-11 as described in table H (compounds I.1.11 to compound I.192.11).

Table 10: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-12 as described in table H (compounds I.1.12 to compound I.192.12).

Table 11: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-13 as described in table H (compounds I.1.13 to compound I.192.13).

Table 12: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-15 as described in table H (compounds I.1.15 to compound I.192.15).

Table 13: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-17 as described in table H (compounds I.1.17 to compound I.192.17).

Table 14: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-18 as described in table H (compounds I.1.18 to compound I.192.18).

Table 15: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-19 as described in table H (compounds I.1.19 to compound I.192.19).

Table 16: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-20 as described in table H (compounds I.1.20 to compound I.192.20).

Table 17: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-21 as described in table H (compounds I.1.21 to compound I.192.21).

Table 18: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-22 as described in table H (compounds I.1.22 to compound I.192.22).

Table 19: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-24 as described in table H (compounds I.1.24 to compound I.192.24).

Table 20: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-25 as described in table H (compounds I.1.25 to compound I.192.25).

Table 21: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-26 as described in table H (compounds I.1.26 to compound I.192.26).

Table 22: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-27 as described in table H (compounds I.1.27 to compound I.192.27).

Table 23: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-28 as described in table H (compounds I.1.28 to compound I.192.28).

Table 24: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-29 as described in table H (compounds I.1.29 to compound I.192.29).

Table 25: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-30 as described in table H (compounds I.1.30 to compound I.192.30).

Table 26: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-31 as described in table H (compounds I.1.31 to compound I.192.31).

Table 27: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-32 as described in table H (compounds I.1.32 to compound I.192.32).

Table 28: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-33 as described in table H (compounds I.1.33 to compound I.192.33).

Table 29: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-34 as described in table H (compounds I.1.34 to compound I.192.34).

Table 30: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-35 as described in table H (compounds I.1.35 to compound I.192.35).

Table 31: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-36 as described in table H (compounds I.1.36 to compound I.192.36).

Table 32: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-37 as described in table H (compounds I.1.37 to compound I.192.37).

Table 33: Compounds I wherein R, R¹ and R² are hydrogen and the meaning of R^{a5}, R^{a6}, R³, R⁴, n and R^{b} in each case have the meaning of one of the combinations selected from lines C-1 to C-192 in table C and wherein the meaning of Het is H-38 as described in table H (compounds I.1.38 to compound I.192.38).

The compounds I and the compositions according to the invention, respectively, are suitable as fungicides. They are distinguished by an outstanding effectiveness against a broad spectrum of phytopathogenic fungi, including soil-borne fungi, which derive especially from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Some are systemically effective and they can be used in crop protection as foliar fungicides, fungicides for seed dressing and soil fungicides. Moreover, they are suitable for controlling harmful fungi, which inter alia occur in wood or roots of plants.

The compounds I and the compositions according to the invention are particularly important in the control of a multitude of phytopathogenic fungi on various cultivated plants, such as cereals, e. g. wheat, rye, barley, triticale, oats or rice; beet, e. g. sugar beet or fodder beet; fruits, such as pomes, stone fruits or soft fruits, e. g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; sweet leaf (also called Stevia); natural rubber plants or ornamental and forestry plants, such as flowers, shrubs, broad-leaved trees or evergreens, e. g. conifers; and on the plant propagation material, such as seeds, and the crop material of these plants.

Preferably, compounds I and compositions thereof, respectively are used for controlling a multitude of fungi on field crops, such as potatoes sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. These young plants may also be protected before transplantation by a total or partial treatment by immersion or pouring.

Preferably, treatment of plant propagation materials with compounds I and compositions thereof, respectively, is used for controlling a multitude of fungi on cereals, such as wheat, rye, barley and oats; rice, corn, cotton and soybeans.

The term “cultivated plants” is to be understood as including plants which have been modified by breeding, mutagenesis or genetic engineering including but not limiting to agricultural biotech products on the market or in development (cf. http://cera-gmc.org/, see GM crop database therein). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e. g. by glycosylation or polymer additions such as prenylated, acetylated or farnesylated moieties or PEG moieties.

Plants that have been modified by breeding, mutagenesis or genetic engineering, e. g. have been rendered tolerant to applications of specific classes of herbicides, such as auxin herbicides such as dicamba or 2,4-D; bleacher herbicides such as hydroxylphenylpyruvate dioxygenase (HPPD) inhibitors or phytoene desaturase (PDS) inhibittors; acetolactate synthase (ALS) inhibitors such as sulfonyl ureas or imidazolinones; enolpyruvylshikimate-3-phosphate synthase (EPSPS) inhibitors, such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i. e. bromoxynil or ioxynil) herbicides as a result of conventional methods of breeding or genetic engineering. Furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are e. g. described in Pest Managem. Sci. 61, 2005, 246; 61, 2005, 258; 61, 2005, 277; 61, 2005, 269; 61, 2005, 286; 64, 2008, 326; 64, 2008, 332; Weed Sci. 57, 2009, 108; Austral. J. Agricult. Res. 58, 2007, 708; Science 316, 2007, 1185; and references quoted therein. Several cultivated plants have been rendered tolerant to herbicides by conventional methods of breeding (mutagenesis), e. g. Clearfield® summer rape (Canola, BASF SE, Germany) being tolerant to imidazolinones, e. g. imazamox, or ExpressSun® sunflowers (DuPont, USA) being tolerant to sulfonyl ureas, e. g. tribenuron. Genetic engineering methods have been used to render cultivated plants such as soybean, cotton, corn, beets and rape, tolerant to herbicides such as glyphosate and glufosinate, some of which are commercially available under the trade names RoundupReady® (glyphosate-tolerant, Monsanto, U.S.A.), Cultivance® (imidazolinone tolerant, BASF SE, Germany) and LibertyLink® (glufosinate-tolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as δ-endotoxins, e. g. CryIA(b), CryIA(c), CryIF, CryIF(a2), CryIIA(b), CryIIIA, CryIIIB(b1) or Cry9c; vegetative insecticidal proteins (VIP), e. g. VIP1, VIP2, VIP3 or VIP3A; insecticidal proteins of bacteria colonizing nematodes, e. g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e. g. WO 02/015701). Further examples of such toxins or genetically modified plants capable of synthesizing such toxins are disclosed, e. g., in EP-A 374 753, WO 93/007278, WO 95/34656, EP-A 427 529, EP-A 451 878, WO 03/18810 und WO 03/52073. The methods for producing such genetically modified plants are generally known to the person skilled in the art and are described, e. g. in the publications mentioned above. These insecticidal proteins contained in the genetically modified plants impart to the plants producing these proteins tolerance to harmful pests from all taxonomic groups of athropods, especially to beetles (Coeloptera), two-winged insects (Diptera), and moths (Lepidoptera) and to nematodes (Nematoda). Genetically modified plants capable to synthesize one or more insecticidal proteins are, e. g., described in the publications mentioned above, and some of which are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme Phosphinothricin-N-Acetyltransferase [PAT]); NuCOTN® 33B (cotton cultivars producing the Cry1Ac toxin), Bollgard® I (cotton cultivars producing the Cry1Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Btl 1 (e. g. Agrisure® CB) and Bt176 from Syngenta Seeds SAS, France, (corn cultivars producing the Cry1Ab toxin and PAT enyzme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bbl toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1F toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e. g. EP-A 392 225), plant disease resistance genes (e. g. potato cultivars, which express resistance genes acting against Phytophthora infestans derived from the mexican wild potato Solanum bulbocastanum) or T4-lysozym (e. g. potato cultivars capable of synthesizing these proteins with increased resistance against bacteria such as Erwinia amylvora). The methods for producing such genetically modified plants are generally known to the person skilled in the art and are described, e. g. in the publications mentioned above.

Furthermore, plants are also covered that are by the use of recombinant DNA techniques capable to synthesize one or more proteins to increase the productivity (e. g. bio mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of substances of content or new substances of content, specifically to improve human or animal nutrition, e. g. oil crops that produce health-promoting long-chain omega-3 fatty acids or unsaturated omega-9 fatty acids (e. g. Nexera® rape, DOW Agro Sciences, Canada).

Furthermore, plants are also covered that contain by the use of recombinant DNA techniques a modified amount of substances of content or new substances of content, specifically to improve raw material production, e. g. potatoes that produce increased amounts of amylopectin (e. g. Amflora® potato, BASF SE, Germany).

The compounds I and compositions thereof, respectively, are particularly suitable for controlling the following plant diseases:

Albugo spp. (white rust) on ornamentals, vegetables (e. g. A. candida) and sunflowers (e. g. A. tragopogonis); Alternaria spp. (Alternaria leaf spot) on vegetables, rape (A. brassicola or brassicae), sugar beets (A. tenuis), fruits, rice, soybeans, potatoes (e. g. A. solani or A. alternata), tomatoes (e. g. A. solani or A. alternata) and wheat; Aphanomyces spp. on sugar beets and vegetables; Ascochyta spp. on cereals and vegetables, e. g. A. tritici (anthracnose) on wheat and A. hordei on barley; Bipolaris and Drechslera spp. (teleomorph: Cochliobolus spp.), e. g. Southern leaf blight (D. maydis) or Northern leaf blight (B. zeicola) on corn, e. g. spot blotch (B. sorokiniana) on cereals and e.g. B. oryzae on rice and turfs; Blumeria (formerly Erysiphe) graminis (powdery mildew) on cereals (e. g. on wheat or barley); Botrytis cinerea (teleomorph: Botryotinia fuckeliana: grey mold) on fruits and berries (e. g. strawberries), vegetables (e. g. lettuce, carrots, celery and cabbages), rape, flowers, vines, forestry plants and wheat; Bremia lactucae (downy mildew) on lettuce; Ceratocystis(syn. Ophiostoma) spp. (rot or wilt) on broad-leaved trees and evergreens, e. g. C. ulmi (Dutch elm disease) on elms; Cercospora spp. (Cercospora leaf spots) on corn (e.g. Gray leaf spot: C. zeae-maydis), rice, sugar beets (e. g. C. beticola), sugar cane, vegetables, coffee, soybeans (e. g. C. sojina or C. kikuchi) and rice; Cladosporium spp. on tomatoes (e. g. C. fulvum: leaf mold) and cereals, e. g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e. g. C. sativus, anamorph: B. sorokiniana) and rice (e. g. C. miyabeanus, anamorph: H. oryzae); Colletotrichum (teleomorph: Glomerella) spp. (anthracnose) on cotton (e. g. C. gossypii), corn (e. g. C. graminicola: Anthracnose stalk rot), soft fruits, potatoes (e. g. C. coccodes, black dot), beans (e. g. C. lindemuthianum) and soybeans (e. g. C. truncatum or C. gloeosporioides); Corticium spp., e. g. C. sasakii (sheath blight) on rice; Corynespora cassiicola (leaf spots) on soybeans and ornamentals; Cycloconium spp., e. g. C. oleaginum on olive trees; Cylindrocarpon spp. (e. g. fruit tree canker or young vine decline, teleomorph: Nectria or Neonectria spp.) on fruit trees, vines (e. g. C. liriodendri teleomorph: Neonectria liriodendr Black Foot Disease) and ornamentals; Dematophora (teleomorph: Rosellinia) necatrix (root and stem rot) on soybeans; Diaporthe spp., e. g. D. phaseolorum (damping off) on soybeans; Drechslera (syn. Helminthosporium, teleomorph: Pyrenophora) spp. on corn, cereals, such as barley (e. g. D. teres, net blotch) and wheat (e. g. D. tritici-repentis: tan spot), rice and turf; Esca (dieback, apoplexy) on vines, caused by Formitiporia (syn. Phellinus) punctata, F. mediterranea, Phaeomoniella chlamydospora (earlier Phaeoacremonium chlamydosporum), Phaeoacremonium aleophilum and/or Botryosphaeria obtusa; E/sinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e. g. E. pisi), such as cucurbits (e. g. E. cichoracearum), cabbages, rape (e. g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e. g. E. turcicum); Fusarium (teleomorph: Gibberella) spp. (wilt, root or stem rot) on various plants, such as F. graminearum or F. culmorum (root rot, scab or head blight) on cereals (e. g. wheat or barley), F. oxysporum on tomatoes, F. solani(f. sp. glycines now syn. F. virguliforme) and F. tucumaniae and F. brasiliense each causing sudden death syndrome on soybeans, and F. verticillioides on corn; Gaeumannomyces graminis (take-all) on cereals (e. g. wheat or barley) and corn; Gibberella spp. on cereals (e. g. G. zeae) and rice (e. g. G. fujikuroi. Bakanae disease); Glomerella cingulata on vines, pome fruits and other plants and G. gossypiion cotton; Grainstaining complex on rice; Guignardia bidwellii (black rot) on vines; Gymnosporangium spp. on rosaceous plants and junipers, e. g. G. sabinae (rust) on pears; Helminthosporium spp. (syn. Drechslera, teleomorph: Cochliobolus) on corn, cereals and rice; Hemlileia spp., e. g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e. g. wheat or barley); Microsphaera diffusa (powdery mildew) on soybeans; Monilinia spp., e. g. M. laxa, M. fructicola and M. fructigena (bloom and twig blight, brown rot) on stone fruits and other rosaceous plants; Mycosphaerella spp. on cereals, bananas, soft fruits and ground nuts, such as e. g. M. gramlinicola (anamorph: Septoria tritici Septoria blotch) on wheat or M. fijiensis (black Sigatoka disease) on bananas; Peronospora spp. (downy mildew) on cabbage (e. g. P. brassicae), rape (e. g. P. parasitica), onions (e. g. P. destructor), tobacco (P. tabacina) and soybeans (e. g. P. manshurica); Phakopsora pachyrhizi and P. meibomiae (soybean rust) on soybeans; Phialophora spp. e. g. on vines (e. g. P. tracheiphila and P. tetraspora) and soybeans (e. g. P. gregata: stem rot); Phoma lingam (root and stem rot) on rape and cabbage and P. betae (root rot, leaf spot and damping-off) on sugar beets; Phomopsis spp. on sunflowers, vines (e. g. P. viticola: can and leaf spot) and soybeans (e. g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e. g. P. capsici), soybeans (e. g. P. megasperma, syn. P. sojae), potatoes and tomatoes (e. g. P. infestans, late blight) and broad-leaved trees (e. g. P. ramorum: sudden oak death); Plasmodiophora brassicae (club root) on cabbage, rape, radish and other plants; Plasmopara spp., e. g. P. viticola (grapevine downy mildew) on vines and P. halstedii on sunflowers; Podosphaera spp. (powdery mildew) on rosaceous plants, hop, pome and soft fruits, e. g. P. leucotricha on apples; Polymyxa spp., e. g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e. g. wheat or barley; Pseudoperonospora (downy mildew) on various plants, e. g. P. cubensis on cucurbits or P. humili on hop; Pseudopezicula tracheiphila (red fire disease or ‘rotbrenner’, anamorph: Phialophora) on vines; Puccinia spp. (rusts) on various plants, e. g. P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye, P. kuehnii(orange rust) on sugar cane and P. asparagion asparagus; Pyrenophora (anamorph: Drechslera) tritici-repentis (tan spot) on wheat or P. teres (net blotch) on barley; Pyricularia spp., e. g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e. g. P. ultimum or P. aphanidermatum); Ramularia spp., e. g. R. collo-cygni (Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e. g. R. solani (root and stem rot) on soybeans, R. solani (sheath blight) on rice or R. cereal/s (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotinia spp. (stem rot or white mold) on vegetables and field crops, such as rape, sunflowers (e. g. S. sclerotiorum) and soybeans (e. g. S. rolfsii or S. sclerotiorum); Septoria spp. on various plants, e. g. S. glycines (brown spot) on soybeans, S. tritici(Septoria blotch) on wheat and S. (syn. Stagonospora) nodorum(Stagonospora blotch) on cereals; Uncinula (syn. Erysiphe) necator (powdery mildew, anamorph: Oldium tuckeri) on vines; Setospaeria spp. (leaf blight) on corn (e. g. S. turcilcum, syn. Helminthosporium turcicum) and turf; Sphacelotheca spp. (smut) on corn, (e. g. S. reiliana: head smut), sorghum und sugar cane; Sphaerotheca fuliginea (powdery mildew) on cucurbits; Spongospora subterranea (powdery scab) on potatoes and thereby transmitted viral diseases; Stagonospora spp. on cereals, e. g. S. nodorum (Stagonospora blotch, teleomorph: Leptosphaeria [syn. Phaeosphaeria]nodorum) on wheat; Synchytrium endobioticum on potatoes (potato wart disease); Taphrina spp., e. g. T. deformans (leaf curl disease) on peaches and T. pruni(plum pocket) on plums; Thielaviopsis spp. (black root rot) on tobacco, pome fruits, vegetables, soybeans and cotton, e. g. T. basicola (syn. Chalara elegans); Tilletia spp. (common bunt or stinking smut) on cereals, such as e. g. T. tritici(syn. T. caries, wheat bunt) and T. controversa (dwarf bunt) on wheat; Typhula incarnata (grey snow mold) on barley or wheat; Urocystis spp., e. g. U. occulta (stem smut) on rye; Uromyces spp. (rust) on vegetables, such as beans (e. g. U. appendiculatus, syn. U. phaseoli) and sugar beets (e. g. U. betae); Ustilago spp. (loose smut) on cereals (e. g. U. nuda and U. avaenae), corn (e. g. U. maydis: corn smut) and sugar cane; Venturia spp. (scab) on apples (e. g. V. inaequalis) and pears; and Verticillium spp. (wilt) on various plants, such as fruits and ornamentals, vines, soft fruits, vegetables and field crops, e. g. V. dahliae on strawberries, rape, potatoes and tomatoes.

The compounds I and compositions thereof, respectively, are also suitable for controlling harmful fungi in the protection of stored products or harvest and in the protection of materials.

The term “protection of materials” is to be understood to denote the protection of technical and non-living materials, such as adhesives, glues, wood, paper and paperboard, textiles, leather, paint dispersions, plastics, coiling lubricants, fiber or fabrics, against the infestation and destruction by harmful microorganisms, such as fungi and bacteria. As to the protection of wood and other materials, the particular attention is paid to the following harmful fungi: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichorma spp., Alternaria spp., Paecilomyces spp. and Zygomycetes such as Mucor spp., and in addition in the protection of stored products and harvest the following yeast fungi are worthy of note: Candida spp. and Saccharomyces cerevisae.

The method of treatment according to the invention can also be used in the field of protecting stored products or harvest against attack of fungi and microorganisms. According to the present invention, the term “stored products” is understood to denote natural substances of plant or animal origin and their processed forms, which have been taken from the natural life cycle and for which long-term protection is desired. Stored products of crop plant origin, such as plants or parts thereof, for example stalks, leafs, tubers, seeds, fruits or grains, can be protected in the freshly harvested state or in processed form, such as pre-dried, moistened, comminuted, ground, pressed or roasted, which process is also known as post-harvest treatment. Also falling under the definition of stored products is timber, whether in the form of crude timber, such as construction timber, electricity pylons and barriers, or in the form of finished articles, such as furniture or objects made from wood. Stored products of animal origin are hides, leather, furs, hairs and the like. The combinations according the present invention can prevent disadvantageous effects such as decay, discoloration or mold.

Preferably “stored products” is understood to denote natural substances of plant origin and their processed forms, more preferably fruits and their processed forms, such as pomes, stone fruits, soft fruits and citrus fruits and their processed forms.

The compounds I and compositions thereof, resepectively, may be used for improving the health of a plant. The invention also relates to a method for improving plant health by treating a plant, its propagation material and/or the locus where the plant is growing or is to grow with an effective amount of compounds I and compositions thereof, respectively.

The term “plant health” is to be understood to denote a condition of the plant and/or its products which is determined by several indicators alone or in combination with each other such as yield (e. g. increased biomass and/or increased content of valuable ingredients), plant vigor (e. g. improved plant growth and/or greener leaves (“greening effect”)), quality (e. g. improved content or composition of certain ingredients) and tolerance to abiotic and/or biotic stress. The above identified indicators for the health condition of a plant may be interdependent or may result from each other.

The compounds of formula I can be present in different crystal modifications whose biological activity may differ. They are likewise subject matter of the present invention.

The compounds I are employed as such or in form of compositions by treating the fungi or the plants, plant propagation materials, such as seeds, soil, surfaces, materials or rooms to be protected from fungal attack with a fungicidally effective amount of the active substances.

The application can be carried out both before and after the infection of the plants, plant propagation materials, such as seeds, soil, surfaces, materials or rooms by the fungi.

Plant propagation materials may be treated with compounds I as such or a composition comprising at least one compound I prophylactically either at or before planting or transplanting.

The invention also relates to agrochemical compositions comprising an auxiliary and at least one compound I according to the invention.

An agrochemical composition comprises a fungicidally effective amount of a compound I.

The term “effective amount” denotes an amount of the composition or of the compounds I, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific compound I used.

The compounds I, their N-oxides and salts can be converted into customary types of agrochemical compositions, e. g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Monograph No. 2, 6^{th} Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e.g. ethanol, propanol, butanol, benzylalcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emusifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines.

Suitable amphoteric surfactants are alkylbetains and imidazolines. Suitable block polymers are block polymers of the A-B or A-B-A type comprising blocks of polyethylene oxide and polypropylene oxide, or of the A-B—C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compound I on the target. Examples are surfactants, mineral or vegetable oils, and other auxilaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), anorganic clays (organically modified or unmodified), polycarboxylates, and silicates.

Suitable bactericides are bronopol and isothiazolinone derivatives such as alkyliso-thiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

Examples for composition types and their preparation are:

i) Water-soluble concentrates (SL, LS)

10-60 wt % of a compound I or a mixture comprising a compound I and 5-15 wt % wetting agent (e.g. alcohol alkoxylates) are dissolved in water and/or in a water-soluble solvent (e.g. alcohols) ad 100 wt %. The active substance dissolves upon dilution with water.

ii) Dispersible concentrates (DC)

5-25 wt % of a compound I and 1-10 wt % dispersant (e. g. polyvinylpyrrolidone) are dissolved in organic solvent (e.g. cyclohexanone) ad 100 wt %. Dilution with water gives a dispersion.

iii) Emulsifiable concentrates (EC)

15-70 wt % of a compound I or a mixture comprising a compound I and 5-10 wt % emulsifiers (e.g. calcium dodecylbenzenesulfonate and castor oil ethoxylate) are dissolved in water-insoluble organic solvent (e.g. aromatic hydrocarbon) ad 100 wt %. Dilution with water gives an emulsion.

iv) Emulsions (EW, EO, ES)

5-40 wt % of a compound I or a mixture comprising a compound I and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into water ad 100 wt % by means of an emulsifying machine and made into a homogeneous emulsion. Dilution with water gives an emulsion.

v) Suspensions (SC, OD, FS)

In an agitated ball mill, 20-60 wt % of a compound I or a mixture comprising a compound I are comminuted with addition of 2-10 wt % dispersants and wetting agents (e.g. sodium lignosulfonate and alcohol ethoxylate), 0.1-2 wt % thickener (e.g. xanthan gum) and water ad 100 wt % to give a fine active substance suspension. Dilution with water gives a stable suspension of the active substance. For FS type composition up to 40 wt % binder (e.g. polyvinylalcohol) is added.

vi) Water-dispersible granules and water-soluble granules (WG, SG)

50-80 wt % of a compound I or a mixture comprising a compound I are ground finely with addition of dispersants and wetting agents (e.g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance.

vii) Water-dispersible powders and water-soluble powders (WP, SP, WS)

50-80 wt % of a compound I or a mixture comprising a compound I are ground in a rotor-stator mill with addition of 1-5 wt % dispersants (e.g. sodium lignosulfonate), 1-3 wt % wetting agents (e.g. alcohol ethoxylate) and solid carrier (e.g. silica gel) ad 100 wt %. Dilution with water gives a stable dispersion or solution of the active substance.

viii) Gel (GW, GF)

In an agitated ball mill, 5-25 wt % of a compound I or a mixture comprising a compound I are comminuted with addition of 3-10 wt % dispersants (e.g. sodium lignosulfonate), 1-5 wt % thickener (e.g. carboxymethylcellulose) and water ad 100 wt % to give a fine suspension of the active substance. Dilution with water gives a stable suspension of the active substance.

ix) Microemulsion (ME)

5-20 wt % of a compound I or a mixture comprising a compound I are added to 5-30 wt % organic solvent blend (e.g. fatty acid dimethylamide and cyclohexanone), 10-25 wt % surfactant blend (e.g. alcohol ethoxylate and arylphenol ethoxylate), and water ad 100%. This mixture is stirred for 1 h to produce spontaneously a thermodynamically stable microemulsion.

x) Microcapsules (CS)

An oil phase comprising 5-50 wt % of a compound I or a mixture comprising a compound I, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radical initiator results in the formation of poly(meth)acrylate microcapsules. Alternatively, an oil phase comprising 5-50 wt % of a compound I or a mixture comprising a compound I according to the invention, 0-40 wt % water insoluble organic solvent (e.g. aromatic hydrocarbon), and an isocyanate monomer (e.g. diphenylmethene-4,4′-diisocyanatae) are dispersed into an aqueous solution of a protective colloid (e.g. polyvinyl alcohol). The addition of a polyamine (e.g. hexamethylenediamine) results in the formation of polyurea microcapsules. The monomers amount to 1-10 wt %. The wt % relate to the total CS composition.

xi) Dustable powders (DP, DS)

1-10 wt % of a compound I or a mixture comprising a compound I are ground finely and mixed intimately with solid carrier (e.g. finely divided kaolin) ad 100 wt %.

xii) Granules (GR, FG)

0.5-30 wt % of a compound I or a mixture comprising a compound I is ground finely and associated with solid carrier (e.g. silicate) ad 100 wt %. Granulation is achieved by extrusion, spray-drying or fluidized bed.

xiii) Ultra-low volume liquids (UL)

1-50 wt % of a compound I or a mixture comprising a compound I are dissolved in organic solvent (e.g. aromatic hydrocarbon) ad 100 wt %.

The compositions types i) to xiii) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, and in particular between 0.5 and 75%, by weight of active substance. The active substances are employed in a purity of from 90% to 100%, preferably from 95% to 100% (according to NMR spectrum).

Solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of treatment of plant propagation materials, particularly seeds. The compositions in question give, after two-to-tenfold dilution, active substance concentrations of from 0.01 to 60% by weight, preferably from 0.1 to 40%, in the ready-to-use preparations. Application can be carried out before or during sowing. Methods for applying compound I and compositions thereof, respectively, on to plant propagation material, especially seeds include dressing, coating, pelleting, dusting, soaking and in-furrow application methods of the propagation material. Preferably, compound I or the compositions thereof, respectively, are applied on to the plant propagation material by a method such that germination is not induced, e. g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha.

In treatment of plant propagation materials such as seeds, e. g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e.g. herbicides, insecticides, fungicides, growth regulators, safeners, biopesticides) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

A pesticide is generally a chemical or biological agent (such as a virus, bacterium, antimicrobial or disinfectant) that through its effect deters, incapacitates, kills or otherwise discourages pests. Target pests can include insects, plant pathogens, weeds, mollusks, birds, mammals, fish, nematodes (roundworms), and microbes that destroy property, cause nuisance, spread disease or are vectors for disease. The term pesticides includes also plant growth regulators that alter the expected growth, flowering, or reproduction rate of plants; defoliants that cause leaves or other foliage to drop from a plant, usually to facilitate harvest; desiccants that promote drying of living tissues, such as unwanted plant tops; plant activators that activate plant physiology for defense of against certain pests; safeners that reduce unwanted herbicidal action of pesticides on crop plants; and plant growth promoters that affect plant physiology to increase plant growth, biomass, yield or any other quality parameter of the harvestable goods of a crop plant.

Biopesticides are typically created by growing and concentrating naturally occurring organisms and/or their metabolites including bacteria and other microbes, fungi, viruses, nematodes, proteins, etc. They are often considered to be important components of integrated pest management (IPM) programmes.

Biopesticides fall into two major classes, microbial and biochemical pesticides:
(1) Microbial pesticides consist of bacteria, fungi or viruses (and often include the metabolites that bacteria and fungi produce). Entomopathogenic nematodes are also classed as microbial pesticides, even though they are multi-cellular.
(2) Biochemical pesticides are naturally occurring substances that control pests or provide other crop protection uses as defined below, but are relatively non-toxic to mammals.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank or any other kind of vessel used for applications (e.g seed treater drums, seed pelleting machinery, knapsack sprayer) and further auxiliaries may be added, if appropriate.

When living microorganisms, such as pesticides from groups L1), L3) and L5), form part of such kit, it must be taken care that choice and amounts of the components (e.g. chemical pesticidal agents) and of the further auxiliaries should not influence the viability of the microbial pesticides in the composition mixed by the user. Especially for bactericides and solvents, compatibility with the respective microbial pesticide has to be taken into account. Consequently, one embodiment of the invention is a kit for preparing a usable pesticidal composition, the kit comprising a) a composition comprising component 1) as defined herein and at least one auxiliary; and b) a composition comprising component 2) as defined herein and at least one auxiliary; and optionally c) a composition comprising at least one auxiliary and optionally a further active component 3) as defined herein.

Mixing the compounds I or the compositions comprising them in the use form as fungicides with other fungicides results in many cases in an expansion of the fungicidal spectrum of activity being obtained or in a prevention of fungicide resistance development. Furthermore, in many cases, synergistic effects are obtained.

The following list of pesticides II (e.g. pesticidally-active substances and biopesticides), in conjunction with which the compounds I can be used, is intended to illustrate the possible combinations but does not limit them:

A) Respiration Inhibitors
Inhibitors of complex III at Qₒ site (e.g. strobilurins): azoxystrobin (A.1.1), coumethoxy-strobin (A.1.2), coumoxystrobin (A.1.3), dimoxystrobin (A.1.4), enestroburin (A.1.5), fenaminstrobin (A.1.6), fenoxystrobin/flufenoxystrobin (A.1.7), fluoxastrobin (A.1.8), kresoxim-methyl (A.1.9), mandestrobin (A.1.10), metominostrobin (A.1.11), orysastrobin (A.1.12), picoxystrobin (A.1.13), pyraclostrobin (A.1.14), pyrametostrobin (A.1.15), pyraoxystrobin (A.1.16), trifloxystrobin (A.1.17) and 2-(2-(3-(2,6-dichlorophenyl)-1-methyl-allylideneaminooxymethyl)-phenyl)-2-methoxyimino-N-methyl-acetamide (A.1.18), pyribencarb (A.1.19), triclopyricarb/chlorodincarb (A.1.20), famoxadone (A.1.21), fenamidone (A.1.22), (Z,2E)-5-[1-(4-chlorophenyl)pyrazol-3-yl]oxy-2-methoxyimino-N,3-dimethyl-pent-3-enamide (A.1.23), (Z,2E)-5-[1-(2,4-difluorophenyl)pyrazol-3-yl]oxy-2-methoxyimino-N,3-dimethyl-pent-3-enamide (A.1.24), (Z,2E)-5-[1-(2,4-dichlorophenyl)pyrazol-3-yl]oxy-2-methoxyimino-N,3-dimethyl-pent-3-enamide (A.1.25), (Z,2E)-5-[1-(2-chloro-4-methyl-phenyl)pyrazol-3-yl]oxy-2-methoxyimino-N,3-dimethyl-pent-3-enamide (A.1.26), (Z,2E)-2-methoxyimino-N,3-dimethyl-5-[1-(p-tolyl)pyrazol-3-yl]oxy-pent-3-enamide (A.1.27), (Z,2E)-5-[1-(2-methyl-4-fluoro-phenyl)pyrazol-3-yl]oxy-2-methoxyimino-N,3-dimethyl-pent-3-enamide (A.1.28), (Z,2E)-2-methoxyimino-N,3-dimethyl-5-[1-[4-(trifluoromethyl)-phenyl]pyrazol-3-yl]oxy-pent-3-enamide (A.1.29), (Z,2E)-5-[1-(3,4-dichlorophenyl)pyrazol-3-yl]oxy-2-methoxyimino-N,3-dimethyl-pent-3-enamide (A.1.30), (Z,2E)-5-[1-(3,4-dimeth-ylphenyl)pyrazol-3-yl]oxy-2-methoxyimino-N,3-dimethyl-pent-3-enamide (A.1.31), (Z,2E)-5-[1-(4-fluoro-3-methyl-phenyl)pyrazol-3-yl]oxy-2-methoxyimino-N,3-d imethyl-pent-3-enamide (A.1.32), (Z,2E)-5-[1-(3-chloro-4-fluoro-phenyl)pyrazol-3-yl]oxy-2-methoxyimino-N,3-dimethyl-pent-3-enamide (A.1.33), (Z,2E)-5-[1-(3-fluoro-4-chloro-phenyl)pyrazol-3-yl]oxy-2-methoxyimino-N,3-dimethyl-pent-3-enamide (A.1.34), (Z,2E)-5-[1-(4-chloro-2-fluoro-phenyl)pyrazol-3-yl]oxy-2-methoxyimino-N,3-dimethyl-pent-3-enamide (A.1.35), (Z,2E)-5-[1-[4-(difluoromethoxy)phenyl]pyrazol-3-yl]oxy-2-methoxyimino-N,3-dimethyl-pent-3-enamide (A.1.36), (Z,2E)-5-[1-(3-cyclopropylphenyl)pyrazol-3-yl]oxy-2-methoxyimino-N,3-dimethyl-pent-3-enamide (A.1.37), (Z,2E)-5-[1-[4-chloro-3-(trifluoromethyl)phenyl]pyrazol-3-yl]oxy-2-methoxyimino-N,3-dimethyl-pent-3-enamide (A.1.38), (Z,2E)-2-methoxyimino-N,3-dimethyl-5-[1-(3,4,5-trifluorophenyl)pyrazol-3-yl]oxy-pent-3-enamide (A.1.39) and (Z,2E)-2-methoxyimino-N,3-dimethyl-5-[1-[4-(trifluoromethylsulfanyl)phenyl]pyrazol-3-yl]oxy-pent-3-enamide (A.1.40);
inhibitors of complex III at Qᵢ site: cyazofamid (A.2.1), amisulbrom (A.2.2), [(3S,6S,7R,8R)-8-benzyl-3-[(3-acetoxy-4-methoxy-pyridine-2-carbonyl)amino]-6-methyl-4,9-dioxo-1,5-dioxonan-7-yl]2-methylpropanoate (A.2.3), [(3S,6S,7R,8R)-8-benzyl-3-[[3-(acet-oxymethoxy)-4-methoxy-pyridine-2-carbonyl]amino]-6-methyl-4,9-dioxo-1,5-dioxonan-7-yl]2-methylpropanoate (A.2.4), [(3S,6S,7R,8R)-8-benzyl-3-[(3-isobutoxycarbonyloxy-4-meth-oxy-pyridine-2-carbonyl)amino]-6-methyl-4,9-dioxo-1,5-dioxonan-7-yl]2-methylpropanoate (A.2.5), [(3S,6S,7R,8R)-8-benzyl-3-[[3-(1,3-benzodioxol-5-ylmethoxy)-4-methoxy-pyridine-2-carbonyl]amino]-6-methyl-4,9-dioxo-1,5-dioxonan-7-yl]2-methylpropanoate (A.2.6); (3S,6S,7R,8R)-3-[[(3-hydroxy-4-methoxy-2-pyridinyl)carbonyl]amino]-6-methyl-4,9-dioxo-8-(phenylmethyl)-1,5-dioxonan-7-yl 2-methylpropanoate (A.2.7);
inhibitors of complex II (e. g. carboxamides): benodanil (A.3.1), benzovindiflupyr (A.3.2), bixafen (A.3.3), boscalid (A.3.4), carboxin (A.3.5), fenfuram (A.3.6), fluopyram (A.3.7), flutolanil (A.3.8), fluxapyroxad (A.3.9), furametpyr (A.3.10), isofetamid (A.3.11), iso-pyrazam (A.3.12), mepronil (A.3.13), oxycarboxin (A.3.14), penflufen (A.3.14), penthiopyrad (A.3.15), sedaxane (A.3.16), tecloftalam (A.3.17), thifluzamide (A.3.18), N-(4′-trifluoromethylthiobiphenyl-2-yl)-3-difluoromethyl-1-methyl-1H-pyrazole-4-carboxamide (A.3.19), N-(2-(1,3,3-trimethyl-butyl)-phenyl)-1,3-dimethyl-5-fluoro-1H-pyrazole-4-carboxamide (A.3.20), 3-(difluoromethyl)-1-methyl-N-(1,1,3-trimethylindan-4-yl)pyrazole-4-carboxamide (A.3.21), 3-(trifluoromethyl)-1-methyl-N-(1,1,3-trimethylindan-4-yl)pyrazole-4-carboxamide (A.3.22), 1,3-dimethyl-N-(1,1,3-trimethylindan-4-yl)pyrazole-4-carboxamide (A.3.23), 3-(trifluoromethyl)-1,5-dimethyl-N-(1,1,3-trimethylindan-4-yl)pyrazole-4-carboxamide (A.3.24), 1,3,5-trimethyl-N-(1,1,3-trimethylindan-4-yl)pyrazole-4-carboxamide (A.3.25), N-(7-fluoro-1,1,3-trimethyl-indan-4-yl)-1,3-dimethyl-pyrazole-4-carboxamide (A.3.26), N-[2-(2,4-dichlorophenyl)-2-methoxy-1-methyl-ethyl]-3-(difluoromethyl)-1-methyl-pyrazole-4-carboxamide (A.3.27);
other respiration inhibitors (e.g. complex I, uncouplers): diflumetorim (A.4.1), (5,8-difluoroquinazolin-4-yl)-{2-[2-fluoro-4-(4-trifluoromethylpyridin-2-yloxy)-phenyl]-ethyl}-amine (A.4.2); nitrophenyl derivates: binapacryl (A.4.3), dinobuton (A.4.4), dinocap (A.4.5), fluazinam (A.4.6); ferimzone (A.4.7); organometal compounds: fentin salts, such as fentin-acetate (A.4.8), fentin chloride (A.4.9) or fentin hydroxide (A.4.10); ametoctradin (A.4.11); and silthiofam (A.4.12);

B) Sterol Biosynthesis Inhibitors (SBI Fungicides)
C14 demethylase inhibitors (DMI fungicides): triazoles: azaconazole (B.1.1), bitertanol (B.1.2), bromuconazole (B.1.3), cyproconazole (B.1.4), difenoconazole (B.1.5), diniconazole (B.1.6), diniconazole-M (B.1.7), epoxiconazole (B.1.8), fenbuconazole (B.1.9), fluquinconazole (B.1.10), flusilazole (B.1.11), flutriafol (B.1.12), hexaconazole (B.1.13), imibenconazole (B.1.14), ipconazole (B.1.15), metconazole (B.1.17), myclobutanil (B.1.18), oxpoconazole (B.1.19), paclobutrazole (B.1.20), penconazole (B.1.21), propiconazole (B.1.22), prothioconazole (B.1.23), simeconazole (B.1.24), tebuconazole (B.1.25), tetraconazole (B.1.26), triadimefon (B.1.27), triadimenol (B.1.28), triticonazole (B.1.29), uniconazole (B.1.30), 1-[rel-(2S;3R)-3-(2-chlorophenyl)-2-(2,4-difluorophenyl)-oxiranylmethyl]-5-thiocyanato-1H-[1,2,4]triazolo (B.1.31), 2-[reb(2S;3R)-3-(2-chlorophenyl)-2-(2,4-difluorophenyl)-oxiranylmethyl]-2H-[1,2,4]triazole-3-thiol (B.1.32), 2-[2-chloro-4-(4-chlorophenoxy)phenyl]-1-(1,2,4-triazol-1-yl)pentan-2-ol (B.1.33), 1-[4-(4-chlorophenoxy)-2-(trifluoromethyl)phenyl]-1-cyclopropyl-2-(1,2,4-triazol-1-yl)ethanol (B.1.34), 2-[4-(4-chlorophenoxy)-2-(trifluoromethyl)phenyl]-1-(1,2,4-triazol-1-yl)butan-2-ol (B.1.35), 2-[2-chloro-4-(4-chlorophenoxy)phenyl]-1-(1,2,4-triazol-1-yl)butan-2-ol (B.1.36), 2-[4-(4-chlorophenoxy)-2-(trifluoromethyl)phenyl]-3-methyl-1-(1,2,4-triazol-1-yl)butan-2-ol (B.1.37), 2-[4-(4-chlorophenoxy)-2-(trifluoromethyl)phenyl]-1-(1,2,4-triazol-1-yl)propan-2-ol (B.1.38), 2-[2-chloro-4-(4-chlorophenoxy)phenyl]-3-methyl-1-(1,2,4-triazol-1-yl)butan-2-ol (B.1.39), 2-[4-(4-chlorophenoxy)-2-(trifluoromethyl)phenyl]-1-(1,2,4-triazol-1-yl)pentan-2-ol (B.1.40), 2-[4-(4-fluorophenoxy)-2-(trifluoromethyl)phenyl]-1-(1,2,4-triazol-1-yl)propan-2-ol (B.1.41); imidazoles: imazalil (B.1.42), pefurazoate (B.1.43), prochloraz (B.1.44), triflumizol (B.1.45); pyrimidines, pyridines and piperazines: fenarimol (B.1.46), nuarimol (B.1.47), pyrifenox (B.1.48), triforine (B.1.49), [3-(4-chloro-2-fluoro-phenyl)-5-(2,4-difluorophenyl)isoxazol-4-yl]-(3-pyridyl)methanol (B.1.50);
Delta14-reductase inhibitors: aldimorph (B.2.1), dodemorph (B.2.2), dodemorph-acetate (B.2.3), fenpropimorph (B.2.4), tridemorph (B.2.5), fenpropidin (B.2.6), piperalin (B.2.7), spiroxamine (B.2.8);
Inhibitors of 3-keto reductase: fenhexamid (B.3.1);

C) Nucleic Acid Synthesis Inhibitors
phenylamides or acyl amino acid fungicides: benalaxyl (C.1.1), benalaxyl-M (C.1.2), kiralaxyl (C.1.3), metalaxyl (C.1.4), metalaxyl-M (mefenoxam, C.1.5), ofurace (C.1.6), oxadixyl (C.1.7);
others: hymexazole (C.2.1), octhilinone (C.2.2), oxolinic acid (C.2.3), bupirimate (C.2.4), 5-fluorocytosine (C.2.5), 5-fluoro-2-(p-tolylmethoxy)pyrimidin-4-amine (C.2.6), 5-fluoro-2-(4-fluorophenylmethoxy)pyrimidin-4-amine (C.2.7);

D) Inhibitors of Cell Division and Cytoskeleton
tubulin inhibitors, such as benzimidazoles, thiophanates: benomyl (D1.1), carbendazim (D1.2), fuberidazole (D1.3), thiabendazole (D1.4), thiophanate-methyl (D1.5); triazolopyrimidines: 5-chloro-7-(4-methylpiperidin-1-yl)-6-(2,4,6-trifluorophenyl)-[1,2,4]triazolo[1,5-a]pyrimidine (D1.6);
other cell division inhibitors: diethofencarb (D2.1), ethaboxam (D2.2), pencycuron (D2.3), fluopicolide (D2.4), zoxamide (D2.5), metrafenone (D2.6), pyriofenone (D2.7);

E) Inhibitors of Amino Acid and Protein Synthesis
methionine synthesis inhibitors (anilino-pyrimidines): cyprodinil (E.1.1), mepanipyrim (E.1.2), pyrimethanil (E.1.3);
protein synthesis inhibitors: blasticidin-S(E.2.1), kasugamycin (E.2.2), kasugamycin hydrochloride-hydrate (E.2.3), mildiomycin (E.2.4), streptomycin (E.2.5), oxytetracyclin (E.2.6), polyoxine (E.2.7), validamycin A (E.2.8);

F) Signal Transduction Inhibitors
MAP/histidine kinase inhibitors: fluoroimid (F.1.1), iprodione (F.1.2), procymidone (F.1.3), vinclozolin (F.1.4), fenpiclonil (F.1.5), fludioxonil (F.1.6);
G protein inhibitors: quinoxyfen (F.2.1);

G) Lipid and Membrane Synthesis Inhibitors
Phospholipid biosynthesis inhibitors: edifenphos (G.1.1), iprobenfos (G.1.2), pyrazo-phos (G.1.3), isoprothiolane (G.1.4);
lipid peroxidation: dicloran (G.2.1), quintozene (G.2.2), tecnazene (G.2.3), tolclofos-methyl (G.2.4), biphenyl (G.2.5), chloroneb (G.2.6), etridiazole (G.2.7);
phospholipid biosynthesis and cell wall deposition: dimethomorph (G.3.1), flumorph (G.3.2), mandipropamid (G.3.3), pyrimorph (G.3.4), benthiavalicarb (G.3.5), iprovalicarb (G.3.6), valifenalate (G.3.7) and N-(1-(1-(4-cyano-phenyl)ethanesulfonyl)-but-2-yl) carbamic acid-(4-fluorophenyl) ester (G.3.8);
compounds affecting cell membrane permeability and fatty acides: propamocarb (G.4.1);
fatty acid amide hydrolase inhibitors: oxathiapiprolin (G.5.1);

H) Inhibitors with Multi Site Action
inorganic active substances: Bordeaux mixture (H.1.1), copper acetate (H.1.2), copper hydroxide (H.1.3), copper oxychloride (H.1.4), basic copper sulfate (H.1.5), sulfur (H.1.6);
thio- and dithiocarbamates: ferbam (H.2.1), mancozeb (H.2.2), maneb (H.2.3), metam (H.2.4), metiram (H.2.5), propineb (H.2.6), thiram (H.2.7), zineb (H.2.8), ziram (H.2.9);
organochlorine compounds (e.g. phthalimides, sulfamides, chloronitriles): anilazine (H.3.1), chlorothalonil (H.3.2), captafol (H.3.3), captan (H.3.4), folpet (H.3.5), dichlofluanid (H.3.6), dichlorophen (H.3.7), hexachlorobenzene (H.3.8), pentachlorphenole (H.3.9) and its salts, phthalide (H.3.10), tolylfluanid (H.3.11), N-(4-chloro-2-nitro-phenyl)-N-ethyl-4-methyl-benzenesulfonamide (H.3.12);
guanidines and others: guanidine (H.4.1), dodine (H.4.2), dodine free base (H.4.3), guazatine (H.4.4), guazatine-acetate (H.4.5), iminoctadine (H.4.6), iminoctadine-triacetate (H.4.7), iminoctadine-tris(albesilate) (H.4.8), dithianon (H.4.9), 2,6-dimethyl-1H,5H-[1,4]dithiino[2,3-c:5,6-c′]dipyrrole-1,3,5,7(2H,6H)-tetraone (H.4.10);

I) Cell Wall Synthesis Inhibitors
inhibitors of glucan synthesis: validamycin (1.1.1), polyoxin B (1.1.2);
melanin synthesis inhibitors: pyroquilon (1.2.1), tricyclazole (1.2.2), carpropamid (1.2.3), dicyclomet (1.2.4), fenoxanil (1.2.5);

J) Plant Defence Inducers
acibenzolar-S-methyl (J.1.1), probenazole (J.1.2), isotianil (J.1.3), tiadinil (J.1.4), prohexadione-calcium (J.1.5); phosphonates: fosetyl (J.1.6), fosetyl-aluminum (J.1.7), phosphorous acid and its salts (J.1.8), potassium or sodium bicarbonate (J.1.9);

K) Unknown Mode of Action
bronopol (K.1.1), chinomethionat (K.1.2), cyflufenamid (K.1.3), cymoxanil (K.1.4), dazomet (K.1.5), debacarb (K.1.6), diclomezine (K.1.7), difenzoquat (K.1.8), difenzoquat-methylsulfate (K.1.9), diphenylamin (K.1.10), fenpyrazamine (K.1.11), flumetover (K.1.12), flusulfamide (K.1.13), flutianil (K.1.14), methasulfocarb (K.1.15), nitrapyrin (K.1.16), nitrothal-isopropyl (K.1.18), oxathiapiprolin (K.1.19), tolprocarb (K.1.20), oxin-copper (K.1.21), proquinazid (K.1.22), tebufloquin (K.1.23), tecloftalam (K.1.24), triazoxide (K.1.25), 2-butoxy-6-iodo-3-propylchromen-4-one (K. 1.26), 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone (K.1.27), 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone (K.1.28), 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone (K.1.29), N-(cyclopropylmethoxyimino-(6-difluoro-methoxy-2,3-difluoro-phenyl)-methyl)-2-phenyl acetamide (K.1.30), N′-(4-(4-chloro-3-trifluoromethyl-phenoxy)-2,5-dimethyl-phenyl)-N-ethyl-N-methyl formamidine (K.1.31), N′-(4-(4-fluoro-3-trifluoromethyl-phenoxy)-2,5-dimethyl-phenyl)-N-ethyl-N-methyl formamidine (K.1.32), N′-(2-methyl-5-trifluoromethyl-4-(3-trimethylsilanyl-prop-oxy)-phenyl)-N-ethyl-N-methyl formamidine (K.1.33), N′-(5-difluoromethyl-2-methyl-4-(3-tri-methylsilanyl-propoxy)-phenyl)-N-ethyl-N-methyl formamidine (K.1.34), methoxy-acetic acid 6-tert-butyl-8-fluoro-2,3-dimethyl-quinolin-4-yl ester (K.1.35), 3-[5-(4-methylphenyl)-2,3-dimethyl-isoxazolidin-3-yl]-pyridine (K.1.36), 3-[5-(4-chloro-phenyl)-2,3-dimethyl-isoxazolidin-3-yl]-pyridine (pyrisoxazole) (K.1.37), N-(6-methoxy-pyridin-3-yl) cyclopropanecarboxylic acid amide (K.1.38), 5-chloro-1-(4,6-dimethoxy-pyrimidin-2-yl)-2-methyl-1H-benzoimidazole (K.1.39), 2-(4-chloro-phenyl)-N-[4-(3,4-dimethoxy-phenyl)-isoxazol-5-yl]-2-prop-2-ynyloxy-acetamide, ethyl (Z)-3-amino-2-cyano-3-phenyl-prop-2-enoate (K.1.40), picarbutrazox (K.1.41), pentyl N-[6-[[(Z)-[(1-methyltetrazol-5-yl)-phenyl-methylene]amino]oxymethyl]-2-pyridyl]carbamate (K.1.42), 2-[2-[(7,8-difluoro-2-methyl-3-quinolyl)oxy]-6-fluoro-phenyl]propan-2-ol (K.1.43), 2-[2-fluoro-6-[(8-fluoro-2-methyl-3-quinolyl)oxy]phen-yl]propan-2-ol (K.1.44), 3-(5-fluoro-3,3,4,4-tetramethyl-3,4-dihydroisoquinolin-1-yl)quinoline (K.1.45), 3-(4,4-difluoro-3,3-dimethyl-3,4-dihydroisoquinolin-1-yl)quinoline (K.1.46), 3-(4,4,5-trifluoro-3,3-dimethyl-3,4-dihydroisoquinolin-1-yl)quinoline (K.1.47);

L) Biopesticides

L1) Microbial pesticides with fungicidal, bactericidal, viricidal and/or plant defense activator activity: Ampelomyces quisqualis, Aspergillus flavus, Aureobasidium pullulans, Bacillus amyloliquefaciens, B. mojavensis, B. pumilus, B. simplex, B. solisalsi, B. subtilis, B. subtilis var. amyloliquefaciens, Candida oleophila, C. saitoana, Clavibacter michiganensis (bacteriophages), Coniothyrium minitans, Cryphonectria parasitica, Cryptococcus albidus, Dilophosphora alopecuri, Fusarium oxysporum, Clonostachys rosea f. catenulate (also named Gliocladium catenulatum), Gliocladium roseum, Lysobacter antibioticus, L. enzymogenes, Metschnikowia fructicola, Microdochium dimerum, Microsphaeropsis ochracea, Muscodor albus, Paenibacillus polymyxa, Pantoea vagans, Phlebiopsis gigantea, Pseudomonas sp., Pseudomonas chloraphis, Pseudozyma flocculosa, Pichia anomala, Pythium oligandrum, Sphaerodes mycoparasitica, Streptomyces griseoviridis, S. lydicus, S. violaceusniger, Talaromyces flavus, Trichoderma asperellum, T. atroviride, T. fertile, T. gamsii, T. harmatum; mixture of T. harzianum and T. viride; mixture of T. polysporum and T. harzianum; T. stromaticum, T. virens (also named Gliocladium virens), T. viride, Typhula phacorrhiza, Ulocladium oudemansii, Verticillium dahlia, zucchini yellow mosaic virus (avirulent strain);

L2) Biochemical pesticides with fungicidal, bactericidal, viricidal and/or plant defense activator activity: chitosan (hydrolysate), harpin protein, laminarin, Menhaden fish oil, natamycin, Plum pox virus coat protein, potassium or sodium bicarbonate, Reynoutria sachlinensis extract, salicylic acid, tea tree oil;

L3) Microbial pesticides with insecticidal, acaricidal, molluscidal and/or nematicidal activity: Agrobacterium radiobacter, Bacillus cereus, B. firmus, B. thuringiensis, B. thuringiensis ssp. aizawai, B. t. ssp. israelensis, B. t. ssp. galleriae, B. t. ssp. kurstaki, B. t. ssp. tenebrionis, Beauveria bassiana, B. brongniartii, Burkholderia sp., Chromobacterium subtsugae, Cydia pomonella granulosis virus, Cryptophlebia leucotreta granulovirus (CrleGV), Isaria fumosorosea, Heterorhabditis bacteriophora, Lecanicillium longisporum, L. muscarium (formerly Verticillium lecanii), Metarhizium anisopliae, M. anisopliae var. acridum, Nomuraea rileyi, Paecilomyces fumosoroseus, P. lilacinus, Paenibacillus popilliae, Pasteuria spp., P. nishizawae, P. penetrans, P. ramose, P. reneformis, P. thornea, P. usgae, Pseudomonas fluorescens, Steinernema carpocapsae, S. feltiae, S. kraussei;

L4) Biochemical pesticides with insecticidal, acaricidal, molluscidal, pheromone and/or nematicidal activity: L-carvone, citral, (E,Z)-7,9-dodecadien-1-yl acetate, ethyl formate, (E,Z)-2,4-ethyl decadienoate (pear ester), (Z,Z,E)-7,11,13-hexadecatrienal, heptyl butyrate, isopropyl myristate, lavanulyl senecioate, cis-jasmone, 2-methyl 1-butanol, methyl eugenol, methyl jasmonate, (E,Z)-2,13-octadecadien-1-ol, (E,Z)-2,13-octadecadien-1-ol acetate, (E,Z)-3,13-octadecadien-1-ol, R-1-octen-3-ol, pentatermanone, potassium silicate, sorbitol actanoate, (E,Z,Z)-3,8,11-tetradecatrienyl acetate, (Z,E)-9,12-tetradecadien-1-yl acetate, Z-7-tetradecen-2-one, Z-9-tetradecen-1-yl acetate, Z-11-tetradecenal, Z-11-tetradecen-1-ol, Acacia negra extract, extract of grapefruit seeds and pulp, extract of Chenopodium ambrosiodae, Catnip oil, Neem oil, Quillay extract, Tagetes oil;

L5) Microbial pesticides with plant stress reducing, plant growth regulator, plant growth promoting and/or yield enhancing activity: Azospirillum amazonense A. brasilense, A. lipoferum, A. irakense, A. halopraeferens, Bradyrhizobium sp., B. elkanii, B. japonicum, B. liaoningense, B. lupini, Delftia acidovorans, Glomus intraradices, Mesorhizobium sp., Paenibacillus alvei, Penicillium bilaiae, Rhizobium leguminosarum bv. phaseolii, R. I. trifolii, R. I. bv. viciae, R. tropici, Sinorhizobium meliloti;

L6) Biochemical pesticides with plant stress reducing, plant growth regulator and/or plant yield enhancing activity: abscisic acid, aluminium silicate (kaolin), 3-decen-2-one, formononetin, genistein, hesperetin, homobrassinlide, humates, jasmonic acid or salts or derivatives thereof, lysophosphatidyl ethanolamine, naringenin, polymeric polyhydroxy acid, Ascophyllum nodosum (Norwegian kelp, Brown kelp) extract and Ecklonia maxima (kelp) extract;

M) Growth Regulators

abscisic acid (M.1.1), amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione (prohexadione-calcium), prohydrojasmon, thidiazuron, triapenthenol, tributyl phosphorotrithioate, 2,3,5-tri-iodobenzoic acid, trinexapac-ethyl and uniconazole;

N) Herbicides
acetamides: acetochlor (N.1.1), alachlor, butachlor, dimethachlor, dimethenamid (N.1.2), flufenacet (N.1.3), mefenacet (N.1.4), metolachlor (N.1.5), metazachlor (N.1.6), napropamide, naproanilide, pethoxamid, pretilachlor, propachlor, thenylchlor;
amino acid derivatives: bilanafos, glyphosate (N.2.1), glufosinate (N.2.2), sulfosate (N.2.3);
aryloxyphenoxypropionates: clodinafop (N.3.1), cyhalofop-butyl, fenoxaprop (N.3.2), fluazifop (N.3.3), haloxyfop (N.3.4), metamifop, propaquizafop, quizalofop, quizalofop-P-tefuryl;
Bipyridyls: diquat, paraquat (N.4.1);
(thio)carbamates: asulam, butylate, carbetamide, desmedipham, dimepiperate, eptam (EPTC), esprocarb, molinate, orbencarb, phenmedipham (N.5.1), prosulfocarb, pyributicarb, thiobencarb, triallate;
cyclohexanediones: butroxydim, clethodim (N.6.1), cycloxydim (N.6.2), profoxydim (N.6.3), sethoxydim (N.6.4), tepraloxydim (N.6.5), tralkoxydim;
dinitroanilines: benfluralin, ethalfluralin, oryzalin, pendimethalin (N.7.1), prodiamine (N.7.2), trifluralin (N.7.3);
diphenyl ethers: acifluorfen (N.8.1), aclonifen, bifenox, diclofop, ethoxyfen, fomesafen, lactofen, oxyfluorfen;
hydroxybenzonitriles: bomoxynil (N.9.1), dichlobenil, ioxynil;
imidazolinones: imazamethabenz, imazamox (N.10.1), imazapic (N.10.2), imazapyr (N.10.3), imazaquin (N.10.4), imazethapyr (N.10.5);
phenoxy acetic acids: clomeprop, 2,4-dichlorophenoxyacetic acid (2,4-D) (N.11.1), 2,4-DB, dichlorprop, MCPA, MCPA-thioethyl, MCPB, Mecoprop;
pyrazines: chloridazon (N.11.1), flufenpyr-ethyl, fluthiacet, norflurazon, pyridate;
pyridines: aminopyralid, clopyralid (N.12.1), diflufenican, dithiopyr, fluridone, fluroxypyr (N.12.2), picloram (N.12.3), picolinafen (N.12.4), thiazopyr;
sulfonyl ureas: amidosulfuron, azimsulfuron, bensulfuron (N.13.1), chlorimuron-ethyl (N.13.2), chlorsulfuron, cinosulfuron, cyclosulfamuron (N.13.3), ethoxysulfuron, flazasulfuron, flucetosulfuron, flupyrsulfuron, foramsulfuron, halosulfuron, imazosulfuron, iodosulfuron (N.13.4), mesosulfuron (N.13.5), metazosulfuron, metsulfuron-methyl (N.13.6), nicosulfuron (N.13.7), oxasulfuron, primisulfuron, prosulfuron, pyrazosulfuron, rimsulfuron (N.13.8), sulfometuron, sulfosulfuron, thifensulfuron, triasulfuron, tribenuron, trifloxysulfuron, triflusulfuron (N.13.9), tritosulfuron, 1-((2-chloro-6-propyl-imidazo[1,2-b]pyridazin-3-yl)sulfonyl)-3-(4,6-dimethoxy-pyrimidin-2-yl)urea;
triazines: ametryn, atrazine (N.14.1), cyanazine, dimethametryn, ethiozin, hexazinone (N.14.2), metamitron, metribuzin, prometryn, simazine, terbuthylazine, terbutryn, triaziflam;
ureas: chlorotoluron, daimuron, diuron (N.15.1), fluometuron, isoproturon, linuron, methabenzthiazuron, tebuthiuron;
other acetolactate synthase inhibitors: bispyribac-sodium, cloransulam-methyl, diclosulam, florasulam (N.16.1), flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone (N.16.2), pyroxsulam;
others: amicarbazone, aminotriazole, anilofos, beflubutamid, benazolin, bencarbazone,benfluresate, benzofenap, bentazone (N.17.1), benzobicyclon, bicyclopyrone, bromacil, bromobutide, butafenacil, butamifos, cafenstrole, carfentrazone, cinidon-ethyl (N.17.2), chlorthal, cinmethylin (N.17.3), clomazone (N.17.4), cumyluron, cyprosulfamide, dicamba (N.17.5), difenzoquat, diflufenzopyr (N.17.6), Drechslera monoceras, endothal, ethofumesate, etobenzanid, fenoxasulfone, fentrazamide, flumiclorac-pentyl, flumioxazin, flupoxam, flurochloridone, flurtamone, indanofan, isoxaben, isoxaflutole, lenacil, propanil, propyzamide, quinclorac (N.17.7), quinmerac (N.17.8), mesotrione (N.17.9), methyl arsonic acid, naptalam, oxadiargyl, oxadiazon, oxaziclomefone, pentoxazone, pinoxaden, pyraclonil, pyraflufen-ethyl, pyrasulfotole, pyrazoxyfen, pyrazolynate, quinoclamine, saflufenacil (N.17.10), sulcotrione (N.17.11), sulfentrazone, terbacil, tefuryltrione, tembotrione, thiencarbazone, topramezone (N.17.12), (3-[2-chloro-4-fluoro-5-(3-methyl-2,6-dioxo-4-trifluoromethyl-3,6-dihydro-2H-pyrimidin-1-yl)-phenoxy]-pyridin-2-yloxy)-acetic acid ethyl ester, 6-amino-5-chloro-2-cyclopropyl-pyrimidine-4-carboxylic acid methyl ester, 6-chloro-3-(2-cyclopropyl-6-methyl-phenoxy)-pyridazin-4-ol, 4-amino-3-chloro-6-(4-chloro-phenyl)-5-fluoro-pyridine-2-carboxylic acid, 4-amino-3-chloro-6-(4-chloro-2-fluoro-3-methoxy-phenyl)-pyridine-2-carboxylic acid methyl ester, and 4-amino-3-chloro-6-(4-chloro-3-dimethylamino-2-fluoro-phenyl)-pyridine-2-carboxylic acid methyl ester;

O) Insecticides
organo(thio)phosphates: acephate (O.1.1), azamethiphos (O.1.2), azinphos-methyl (O.1.3), chlorpyrifos (O.1.4), chlorpyrifos-methyl (O.1.5), chlorfenvinphos (O.1.6), diazinon (O.1.7), dichlorvos (O.1.8), dicrotophos (O.1.9), dimethoate (O.1.10), disulfoton (O.1.11), ethion (O.1.12), fenitrothion (O.1.13), fenthion (O.1.14), isoxathion (O.1.15), malathion (O.1.16), methamidophos (O.1.17), methidathion (O.1.18), methyl-parathion (O.1.19), mevinphos (O.1.20), monocrotophos (O.1.21), oxydemeton-methyl (O.1.22), paraoxon (O.1.23), parathion (O.1.24), phenthoate (O.1.25), phosalone (O.1.26), phosmet (O.1.27), phosphamidon (O.1.28), phorate (O.1.29), phoxim (O.1.30), pirimiphos-methyl (O.1.31), profenofos (O.1.32), prothiofos (O.1.33), sulprophos (O.1.34), tetrachlorvinphos (O.1.35), terbufos (O.1.36), triazophos (O.1.37), trichlorfon (O.1.38);
carbamates: alanycarb (O.2.1), aldicarb (O.2.2), bendiocarb (O.2.3), benfuracarb (O.2.4), carbaryl (O.2.5), carbofuran (O.2.6), carbosulfan (O.2.7), fenoxycarb (O.2.8), furathiocarb (O.2.9), methiocarb (O.2.10), methomyl (O.2.11), oxamyl (O.2.12), pirimicarb (O.2.13), propoxur (O.2.14), thiodicarb (O.2.15), triazamate (O.2.16);
pyrethroids: allethrin (O.3.1), bifenthrin (O.3.2), cyfluthrin (O.3.3), cyhalothrin (O.3.4), cyphenothrin (O.3.5), cypermethrin (O.3.6), alpha-cypermethrin (O.3.7), beta-cypermethrin (O.3.8), zeta-cypermethrin (O.3.9), deltamethrin (O.3.10), esfenvalerate (O.3.11), etofenprox (O.3.11), fenpropathrin (O.3.12), fenvalerate (O.3.13), imiprothrin (O.3.14), lambda-cyhalothrin (O.3.15), permethrin (O.3.16), prallethrin (O.3.17), pyrethrin I and II (O.3.18), resmethrin (O.3.19), silafluofen (O.3.20), tau-fluvalinate (O.3.21), tefluthrin (O.3.22), tetramethrin (O.3.23), tralomethrin (O.3.24), transfluthrin (O.3.25), profluthrin (O.3.26), dimefluthrin (O.3.27);
insect growth regulators: a) chitin synthesis inhibitors: benzoylureas: chlorfluazuron (O.4.1), cyramazin (O.4.2), diflubenzuron (O.4.3), flucycloxuron (O.4.4), flufenoxuron (O.4.5), hexaflumuron (O.4.6), lufenuron (O.4.7), novaluron (O.4.8), teflubenzuron (O.4.9), triflumuron (O.4.10); buprofezin (O.4.11), diofenolan (O.4.12), hexythiazox (O.4.13), etoxazole (O.4.14), clofentazine (O.4.15); b) ecdysone antagonists: halofenozide (O.4.16), methoxyfenozide (O.4.17), tebufenozide (O.4.18), azadirachtin (O.4.19); c) juvenoids: pyriproxyfen (O.4.20), methoprene (O.4.21), fenoxycarb (O.4.22); d) lipid biosynthesis inhibitors: spirodiclofen (O.4.23), spiromesifen (O.4.24), spirotetramat (O.4.24);
nicotinic receptor agonists/antagonists compounds: clothianidin (O.5.1), dinotefuran (O.5.2), flupyradifurone (O.5.3), imidacloprid (O.5.4), thiamethoxam (O.5.5), nitenpyram (O.5.6), acetamiprid (O.5.7), thiacloprid (O.5.8), 1-2-chloro-thiazol-5-ylmethyl)-2-nitrimino-3,5-dimethyl-[1,3,5]triazinane (O.5.9);
GABA antagonist compounds: endosulfan (O.6.19, ethiprole (O.6.2), fipronil (O.6.3), vaniliprole (O.6.4), pyrafluprole (O.6.5), pyriprole (O.6.6), 5-amino-1-(2,6-dichloro-4-methyl-phenyl)-4-sulfinamoyl-1H-pyrazole-3-carbothioic acid amide (O.6.7);
macrocyclic lactone insecticides: abamectin (O.7.1), emamectin (O.7.2), milbemectin (O.7.3), lepimectin (O.7.4), spinosad (O.7.5), spinetoram (O.7.6);
mitochondrial electron transport inhibitor (METI) I acaricides: fenazaquin (O.8.1), pyridaben (O.8.2), tebufenpyrad (O.8.3), tolfenpyrad (O.8.4), flufenerim (O.8.5);
METI II and III compounds: acequinocyl (O.9.1), fluacyprim (O.9.2), hydramethylnon (O.9.3);
Uncouplers: chlorfenapyr (O.10.1);
oxidative phosphorylation inhibitors: cyhexatin (O.11.1), diafenthiuron (O.11.2), fenbutatin oxide (O.11.3), propargite (O.11.4);
moulting disruptor compounds: cryomazine (O.12.1);
mixed function oxidase inhibitors: piperonyl butoxide (O.13.1);
sodium channel blockers: indoxacarb (O.14.1), metaflumizone (O.14.2);
ryanodine receptor inhibitors: chlorantraniliprole (O.15.1), cyantraniliprole (O.15.2), flubendiamide (O.15.3), N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide (O.15.4); N-[4-chloro-2-[(diethyl-lambda-4-sulfanylidene)carbamoyl]-6-methyl-phenyl]-2-(3-chloro-2-pyridyl)-5-(trifluoromethyl)pyrazole-3-carboxamide (O.15.5); N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide (O.15.6); N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide (O.15.7); N-[4,6-dichloro-2-[(diethyl-lambda-4-sulfanylidene)carbamoyl]-phenyl]-2-(3-chloro-2-pyridyl)-5-(difluoromethyl)pyrazole-3-carboxamide (O.15.8); N-[4,6-dibromo-2-[(di-2-propyl-lambda-4-sulfanylidene)carbamoyl]-phenyl]-2-(3-chloro-2-pyridyl)-5-(trifluoromethyl)pyrazole-3-carboxamide (O.15.9); N-[4-chloro-2-[(di-2-propyl-lambda-4-sulfanylidene)carbamoyl]-6-cyano-phenyl]-2-(3-chloro-2-pyridyl)-5-(trifluoromethyl)pyrazole-3-carboxamide (O.15.10); N-[4,6-dibromo-2-[(diethyl-lambda-4-sulfanylidene)carbamoyl]-phenyl]-2-(3-chloro-2-pyridyl)-5-(trifluoromethyl)pyrazole-3-carboxamide (O.15.11);
others: benclothiaz (O.16.1), bifenazate (O.16.2), artap (O.16.3), flonicamid (O.16.4), pyridalyl (O.16.5), pymetrozine (O.16.6), sulfur (O.16.7), thiocyclam (O.16.8), cyenopyrafen (O.16.9), flupyrazofos (O.16.10), cyflumetofen (O.16.11), amidoflumet (O.16.12), imicyafos (O.16.13), bistrifluron (O.16.14), pyrifluquinazon (O.16.15) and 1,1′-[(3S,4R,4aR,6S,6aS,12R,12aS,12bS)-4-[[(2-cyclopropylacetyl)oxy]methyl]-1,3,4,4a,5,6,6a,12,12a,12b-decahydro-12-hydroxy-4,6a,12b-trimethyl-11-oxo-9-(3-pyridinyl)-2H,11H-naphtho[2,1-b]pyrano[3,4-e]pyran-3,6-diyl]cyclopropaneacetic acid ester (O.16.16).

The present invention furthermore relates to agrochemical compositions comprising a mixture of at least one compound I (component 1) and at least one further active substance useful for plant protection, e. g. selected from the groups A) to O) (component 2), in particular one further fungicide, e. g. one or more fungicide from the groups A) to K), as described above, and if desired one suitable solvent or solid carrier. Those mixtures are of particular interest, since many of them at the same application rate show higher efficiencies against harmful fungi. Furthermore, combating harmful fungi with a mixture of compounds I and at least one fungicide from groups A) to L), as described above, is more efficient than combating those fungi with individual compounds I or individual fungicides from groups A) to L). By applying compounds I together with at least one active substance from groups A) to O) a synergistic effect can be obtained, i.e. more then simple addition of the individual effects is obtained (synergistic mixtures).

This can be obtained by applying the compounds I and at least one further active substance simultaneously, either jointly (e. g. as tank-mix) or separately, or in succession, wherein the time interval between the individual applications is selected to ensure that the active substance applied first still occurs at the site of action in a sufficient amount at the time of application of the further active substance(s). The order of application is not essential for working of the present invention.

When applying a compound I and a pesticide II sequentially the time between both applications may vary e.g. between 2 hours to 7 days. Also a broader range is possible ranging from 0.25 hour to 30 days, preferably from 0.5 hour to 14 days, particularly from 1 hour to 7 days or from 1.5 hours to 5 days, even more preferred from 2 hours to 1 day. In case of a mixture comprising a pesticide II selected from group L), it is preferred that the pesticide II is applied as last treatment.

According to the invention, the solid material (dry matter) of the biopesticides (with the exception of oils such as Neem oil, Tagetes oil, etc.) are considered as active components (e.g. to be obtained after drying or evaporation of the extraction medium or the suspension medium in case of liquid formulations of the microbial pesticides).

In accordance with the present invention, the weight ratios and percentages used herein for a biological extract such as Quillay extract are based on the total weight of the dry content (solid material) of the respective extract(s).

The total weight ratios of compositions comprising at least one microbial pesticide in the form of viable microbial cells including dormant forms, can be determined using the amount of CFU of the respective microorganism to calculate the total weight of the respective active component with the following equation that 1×10⁹ CFU equals one gram of total weight of the respective active component. Colony forming unit is measure of viable microbial cells, in particular fungal and bacterial cells. In addition, here “CFU” may also be understood as the number of (juvenile) individual nematodes in case of (entomopathogenic) nematode biopesticides, such as Steinernema feltiae.

In the binary mixtures and compositions according to the invention the weight ratio of the component 1) and the component 2) generally depends from the properties of the active components used, usually it is in the range of from 1:100 to 100:1, regularly in the range of from 1:50 to 50:1, preferably in the range of from 1:20 to 20:1, more preferably in the range of from 1:10 to 10:1, even more preferably in the range of from 1:4 to 4:1 and in particular in the range of from 1:2 to 2:1.

According to a further embodiments of the binary mixtures and compositions, the weight ratio of the component 1) and the component 2) usually is in the range of from 1000:1 to 1:1, often in the range of from 100: 1 to 1:1, regularly in the range of from 50:1 to 1:1, preferably in the range of from 20:1 to 1:1, more preferably in the range of from 10:1 to 1:1, even more preferably in the range of from 4:1 to 1:1 and in particular in the range of from 2:1 to 1:1.

According to a further embodiments of the binary mixtures and compositions, the weight ratio of the component 1) and the component 2) usually is in the range of from 1:1 to 1:1000, often in the range of from 1:1 to 1:100, regularly in the range of from 1:1 to 1:50, preferably in the range of from 1:1 to 1:20, more preferably in the range of from 1:1 to 1:10, even more preferably in the range of from 1:1 to 1:4 and in particular in the range of from 1:1 to 1:2.

In the ternary mixtures, i.e. compositions according to the invention comprising the component 1) and component 2) and a pesticide III (component 3), the weight ratio of component 1) and component 2) depends from the properties of the active substances used, usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:4 to 4:1, and the weight ratio of component 1) and component 3) usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:4 to 4:1.

Any further active components are, if desired, added in a ratio of from 20:1 to 1:20 to the component 1).

These ratios are also suitable for inventive mixtures applied by seed treatment.

Preference is also given to mixtures comprising as component 2) at least one active substance selected from group A), which is particularly selected from (A.1.1), (A.1.4), (A.1.8), (A.1.9), (A.1.12), (A.1.13), (A.1.14), (A.1.17), (A.1.19), (A.1.21), (A.2.1), (A.2.2), (A.3.2), (A.3.3), (A.3.4), (A.3.7), (A.3.8), (A.3.9), (A.3.12), (A.3.14), (A.3.15), (A.3.16), (A.3.19), (A.3.20), (A.3.21), (A.3.22), (A.3.23), (A.3.24), (A.3.25), (A.3.26), (A.3.27); (A.4.5), (A.4.6), (A.4.8), (A.4.9) and (A.4.11).

Preference is given to mixtures as component 2) at least one active substance selected from group B), which is particularly selected from (B.1.4), (B.1.5), diniconazole (B.1.6), (B.1.8), (B.1.10), (B.1.11), (B.1.12), (B.1.17), (B.1.18), (B.1.21), (B.1.22), (B.1.23), (B.1.25), (B.1.26), (B.1.27), (B.1.28), (B.1.29), (B.1.31), (B.1.32), (B.1.33), (B.1.34), (B.1.35), (B.1.36), (B.1.37), (B.1.38), (B.1.39), (B.1.40), (B.1.41), (B.1.42), (B.1.44), (B.1.46), (B.1.49) and (B.1.[TJ1]50); (B.2.2), (B.2.4), (B.2.5), (B.2.6), piperalin (B.2.7), (B.2.8); and (B.3.1).

Preference is given to mixtures comprising as component 2) at least one active substance selected from group C), which is particularly selected from (C.1.4), C.1.5), (C.1.6), and (C.2.4).

Preference is given to mixtures comprising as component 2) at least one active substance selected from group D), which is particularly selected from (D1.1), (D1.2), (D1.4), (D1.5); (D2.2), (D2.4), (D2.5), (D2.6) and (D2.7);

Preference is also given to mixtures comprising as component 2) at least one active substance selected from group E), which is particularly selected from (E.1.1), (E.1.2), and (E.1.3);

Preference is also given to mixtures comprising as component 2) at least one active substance selected from group F), which is particularly selected from (F.1.2), (F.1.4), (F.1.5), (F.1.6) and (F.2.1).

Preference is also given to mixtures as component 2) at least one active substance selected from group G), which is particularly selected from (G.3.1), (G.3.2), (G.3.3), (G.3.4), (G.3.5), (G.3.6), (G.4.1) and (G.5.1).

Preference is also given to mixtures comprising as component 2) at least one active substance selected from group H), which is and particularly selected from (H.1.2), (H.1.3), copper oxychloride (H.1.4), (H.1.5), (H.1.6); (H.2.2), (H.2.5), (H.2.7), (H.3.2), (H.3.3), (H.3.4), (H.3.5), (H.3.6), (H.3.12); (H.4.2), (H.4.6), dithianon (H.4.9) and (H.4.10).

Preference is also given to mixtures comprising as component 2) at least one active substance selected from group I), which is particularly selected from (1.2.3) and (1.2.5).

Preference is also given to mixtures comprising as component 2) at least one active substance selected from group J), which is particularly selected from (J.1.1), (J.1.2), (J.1.3), (J.1.4), (J.1.6), (J.1.7), (J.1.8) and (J.1.9).

Preference is also given to mixtures comprising as component 2) at least one active substance selected from group K), which is particularly selected from (K.1.4), (K.1.5), (K.1.8), (K.1.12), (K.1.14), (K.1.15), (K.1.19) and (K.1.22).

The biopesticides from group L) of pesticides II, their preparation and their pesticidal activity e.g. against harmful fungi or insects are known (e-Pesticide Manual V 5.2 (ISBN 978 1 901396 85 0) (2008-2011); http://www.epa.gov/opp00001/biopesticides/, see product lists therein; http://www.omri.org/omri-lists, see lists therein; Bio-Pesticides Database BPDB http://sitem.herts.ac.uk/aeru/bpdb/, see A to Z link therein).

The biopesticides from group L1) and/or L2) may also have insecticidal, acaricidal, molluscidal, pheromone, nematicidal, plant stress reducing, plant growth regulator, plant growth promoting and/or yield enhancing activity. The biopesticides from group L3) and/or L4) may also have fungicidal, bactericidal, viricidal, plant defense activator, plant stress reducing, plant growth regulator, plant growth promoting and/or yield enhancing activity. The biopesticides from group L5) and/or L6) may also have fungicidal, bactericidal, viricidal, plant defense activator, insecticidal, acaricidal, molluscidal, pheromone and/or nematicidal activity. Many of these biopesticides are registered and/or are commercially available: aluminium silicate (Screen™ Duo from Certis LLC, USA), Agrobacterium radiobacter K1026 (e.g. NoGall® from Becker Underwood Pty Ltd., Australia), A. radiobacter K84 (Nature 280, 697-699, 1979; e.g. GallTroll® from AG Biochem, Inc., C, USA), Ampelomyces quisqualis M-10 (e.g. AQ 10® from Intrachem Bio GmbH & Co. KG, Germany), Ascophyllum nodosum (Norwegian kelp, Brown kelp) extract or filtrate (e.g. ORKA GOLD from Becker Underwood, South Africa; or Goemar® from Laboratoires Goemar, France), Aspergillus flavus NRRL 21882 isolated from a peanut in Georgia in 1991 by the USDA, National Peanut Research Laboratory (e.g. in Afla-Guard® from Syngenta, CH), mixtures of Aureobasidium pullulans DSM14940 and DSM 14941 (e.g. blastospores in BlossomProtect® from bio-ferm GmbH, Germany), Azospirillum brasilense XOH (e.g. AZOS from Xtreme Gardening, USA or RTI Reforestation Technologies International; USA), Bacillus amyloliquefaciens FZB42 (e.g. in RhizoVital® 42 from AbiTEP GmbH, Berlin, Germany), B. amyloliquefaciens IN937a (J. Microbiol. Biotechnol. 17(2), 280-286, 2007; e.g. in BioYield® from Gustafson LLC, TX, USA), B. amyloliquefaciens IT-45 (CNCM 1-3800) (e.g. Rhizocell C from ITHEC, France), B. amyloliquefaciens subsp. plantarum MB1600 (NRRL B-50595, deposited at United States Department of Agriculture) (e.g. Integral®, Subtilex® NG from Becker Underwood, USA), B. cereus CNCM 1-1562 (U.S. Pat. No. 6,406,690), B. firmus CNCM 1-1582 (WO 2009/126473, WO 2009/124707, U.S. Pat. No. 6,406,690; Votivo® from Bayer Crop Science LP, USA), B. pumilus GB34 (ATCC 700814; e.g. in YieldShield® from Gustafson LLC, TX, USA), and Bacillus pumilus KFP9F (NRRL B-50754) (e.g. in BAC-UP or FUSION—P from Becker Underwood South Africa), B. pumilus QST 2808 (NRRL B-30087) (e.g. Sonata® and Ballad® Plus from AgraQuest Inc., USA), B. subtilis GB03 (e.g. Kodiak® or BioYield® from Gustafson, Inc., USA; or Companion® from Growth Products, Ltd., White Plains, N.Y. 10603, USA), B. subtilis GB07 (Epic® from Gustafson, Inc., USA), B. subtilis QST-713 (NRRL B-21661 in Rhapsody®, Serenade® MAX and Serenade® ASO from AgraQuest Inc., USA), B. subtilis var. amyloliquefaciens FZB24 (e.g. Taegro® from Novozyme Biologicals, Inc., USA), B. subtilis var. amyloliquefaciens D747 (e.g. Double Nickel 55 from Certis LLC, USA), B. thuringiensis ssp. aizawai ABTS-1857 (e.g. in XenTari® from BioFa AG, Munsingen, Germany), B. t. ssp. aizawai SAN 401 I, ABG-6305 and ABG-6346, Bacillus t. ssp. israelensis AM65-52 (e.g. in VectoBac® from Valent BioSciences, IL, USA), Bacillus thuringiensis ssp. kurstaki SB4 (NRRL B-50753; e.g. Beta Pro® from Becker Underwood, South Africa), B. t. ssp. kurstaki ABTS-351 identical to HD-1 (ATCC SD-1275; e.g. in Dipel® DF from Valent BioSciences, IL, USA), B. t. ssp. kurstaki EG 2348 (e.g. in Lepinox® or Rapax® from CBC (Europe) S.r.I., Italy), B. t. ssp. tenebrionis DSM 2803 (EP 0 585 215 B1; identical to NRRL B-15939; Mycogen Corp.), B. t. ssp. tenebrionis NB-125 (DSM 5526; EP 0 585 215 B1; also referred to as SAN 418 I or ABG-6479; former production strain of Novo-Nordisk), B. t. ssp. tenebrionis NB-176 (or NB-176-1) a gamma-irridated, induced high-yielding mutant of strain NB-125 (DSM 5480; EP 585 215 B1; Novodor® from Valent BioSciences, Switzerland), Beauveria bassiana ATCC 74040 (e.g. in Naturalis® from CBC (Europe) S.r.l., Italy), B. bassiana DSM 12256 (US 200020031495; e.g. BioExpert® SC from Live Sytems Technology S.A., Colombia), B. bassiana GHA (BotaniGard® 22WGP from Laverlam Int. Corp., USA), B. bassiana PPRI 5339 (ARSEF number 5339 in the USDA ARS collection of entomopathogenic fungal cultures; NRRL 50757) (e.g. BroadBand® from Becker Underwood, South Africa), B. brongniartii (e.g. in Melocont® from Agrifutur, Agrianello, Italy, for control of cockchafer; J. Appl. Microbiol. 100(5),1063-72, 2006), Bradyrhizobium sp. (e.g. Vault® from Becker Underwood, USA), B. japonicum (e.g. VAULT® from Becker Underwood, USA), Candida oleophila 1-182 (NRRL Y-18846; e.g. Aspire® from Ecogen Inc., USA, Phytoparasitica 23(3), 231-234, 1995), C. oleophila strain O (NRRL Y-2317; Biological Control 51, 403-408, 2009), Candida saitoana (e.g. Biocure® (in mixture with lysozyme) and BioCoat® from Micro Flo Company, USA (BASF SE) and Arysta), Chitosan (e.g. Armour-Zen® from BotriZen Ltd., NZ), Clonostachys rosea f. catenulata, also named Gliocladium catenulatum (e.g. isolate J 1446: Prestop® from Verdera Oy, Finland), Chromobacterium subtsugae PRAA4-1 isolated from soil under an eastern hemlock (Tsuga canadensis) in the Catoctin Mountain region of central Maryland (e.g. in GRANDEVO from Marrone Bio Innovations, USA), Coniothyrium minitans CON/M/91-08 (e.g. Contans® WG from Prophyta, Germany), Cryphonectria parasitica (e.g. Endothia parasitica from CNICM, France), Cryptococcus albidus (e.g. YIELD PLUS® from Anchor Bio-Technologies, South Africa), Cryptophlebia leucotreta granulovirus (CrleGV) (e.g. in CRYPTEX from Adermatt Biocontrol, Switzerland), Cydia pomonella granulovirus (CpGV) V03 (DSM GV-0006; e.g. in MADEX Max from Andermatt Biocontrol, Switzerland), CpGV V22 (DSM GV-0014; e.g. in MADEX Twin from Adermatt Biocontrol, Switzerland), Delftia acidovorans RAY209 (ATCC PTA-4249; WO 2003/57861; e.g. in BIOBOOST from Brett Young, Winnipeg, Canada), Dilophosphora alopecuri (Twist Fungus from Becker Underwood, Australia), Ecklonia maxima (kelp) extract (e.g. KELPAK SL from Kelp Products Ltd, South Africa), formononetin (e.g. in MYCONATE from Plant Health Care plc, U.K.), Fusarium oxysporum (e.g. BIOFOX® from S.I.A.P.A., Italy, FUSACLEAN® from Natural Plant Protection, France), Glomus intraradices (e.g. MYC 4000 from ITHEC, France), Glomus intraradices RTI-801 (e.g. MYKOS from Xtreme Gardening, USA or RTI Reforestation Technologies International; USA), grapefruit seeds and pulp extract (e.g. BC-1000 from Chemie S.A., Chile), harpin (alpha-beta) protein (e.g. MESSENGER or HARP—N-Tek from Plant Health Care plc, U.K.; Science 257, 1-132, 1992), Heterorhabditis bacteriophaga (e.g. Nemasys® G from Becker Underwood Ltd., UK), Isaria fumosorosea Apopka-97 (ATCC 20874) (PFR-97™ from Certis LLC, USA), cis-jasmone (U.S. Pat. No. 8,221,736), laminarin (e.g. in VACCIPLANT from Laboratoires Goemar, St. Malo, France or Stahler SA, Switzerland), Lecanicillium longisporum KV42 and KV71 (e.g. VERTALEC® from Koppert BV, Netherlands), L. muscarium KV01 (formerly Verticillium lecanii) (e.g. MYCOTAL from Koppert BV, Netherlands), Lysobacter antibioticus 13-1 (Biological Control 45, 288-296, 2008), L. antibioticus HS124 (Curr. Microbiol. 59(6), 608-615, 2009), L. enzymogenes 3.1T8 (Microbiol. Res. 158, 107-115; Biological Control 31(2), 145-154, 2004), Metarhizium anisopliae var. acridum IMI 330189 (isolated from Ornithacris cavroisi in Niger; also NRRL 50758) (e.g. GREEN MUSCLE® from Becker Underwood, South Africa), M. a. var. acridum FI-985 (e.g. GREEN GUARD® SC from Becker Underwood Pty Ltd, Australia), M. anisopliae Fl-1045 (e.g. BIOCANE® from Becker Underwood Pty Ltd, Australia), M. anisopliae F52 (DSM 3884, ATCC 90448; e.g. MET52® Novozymes Biologicals BioAg Group, Canada), M. anisopliae ICIPE 69 (e.g. METATHRIPOL from ICIPE, Nairobe, Kenya), Metschnikowia fructicola (NRRL Y-30752; e.g. SHEMER® from Agrogreen, Israel, now distributed by Bayer CropSciences, Germany; U.S. Pat. No. 6,994,849), Microdochium dimerum (e.g. ANTIBOT® from Agrauxine, France), Microsphaeropsis ochracea P130A (ATCC 74412 isolated from apple leaves from an abandoned orchard, St-Joseph-du-Lac, Quebec, Canada in 1993; Mycologia 94(2), 297-301, 2002), Muscodor albus QST 20799 originally isolated from the bark of a cinnamon tree in Honduras (e.g. in development products Muscudor™ or QRD300 from AgraQuest, USA), Neem oil (e.g. TRILOGY®, TRIACT® 70 EC from Certis LLC, USA), Nomuraea rileyi strains SA86101, GU87401, SR86151, CG128 and VA9101, Paecilomyces fumosoroseus FE 9901 (e.g. NO FLY™ from Natural Industries, Inc., USA), P. lilacinus 251 (e.g. in BioAct®/MeloCon® from Prophyta, Germany; Crop Protection 27, 352-361, 2008; originally isolated from infected nematode eggs in the Philippines), P. lilacinus DSM 15169 (e.g. NEMATA® SC from Live Systems Technology S.A., Colombia), P. lilacinus BCP2 (NRRL 50756; e.g. PL GOLD from Becker Underwood BioAg SA Ltd, South Africa), mixture of Paenibacillus alvei NAS6G6 (NRRL B-50755), Pantoea vagans (formerly agglomerans) C9-1 (originally isolated in 1994 from apple stem tissue; BlightBan C9-1® from NuFrams America Inc., USA, for control of fire blight in apple; J. Bacteriol. 192(24) 6486-6487, 2010), Pasteuria spp. ATCC PTA-9643 (WO 2010/085795), Pasteuria spp. ATCC SD-5832 (WO 2012/064527), P. nishizawae (WO 2010/80169), P. penetrans (U.S. Pat. No. 5,248,500), P. ramose (WO 2010/80619), P. thornea (WO 2010/80169), P. usgae (WO 2010/80169), Penicillium bilaiae (e.g. Jump Start® from Novozymes Biologicals BioAg Group, Canada, originally isolated from soil in southern Alberta; Fertilizer Res. 39, 97-103, 1994), Phlebiopsis gigantea (e.g. RotStop® from Verdera Oy, Finland), Pichia anomala WRL-076 (NRRL Y-30842; U.S. Pat. No. 8,206,972), potassium bicarbonate (e.g. Amicarb® fromm Stahler SA, Switzerland), potassium silicate (e.g. Sil-MATRIX™ from Certis LLC, USA), Pseudozyma flocculosa PF-A22 UL (e.g. Sporodex® from Plant Products Co. Ltd., Canada), Pseudomonas sp. DSM 13134 (WO 2001/40441, e.g. in PRORADIX from Sourcon Padena GmbH & Co. KG, Hechinger Str. 262, 72072 Tubingen, Germany), P. chloraphis MA 342 (e.g. in CERALL or CEDEMON from BioAgri AB, Uppsala, Sweden), P. fluorescens CL 145A (e.g. in ZEQUANOX from Marrone Biolnnovations, Davis, Calif., USA; J. Invertebr. Pathol. 113(1):104-14, 2013), Pythium oligandrum DV 74 (ATCC 38472; e.g. POLYVERSUM® from Remeslo SSRO, Biopreparaty, Czech Rep. and GOWAN, USA; US 2013/0035230), Reynoutria sachlinensis extract (e.g. REGALIA® SC from Marrone Biolnnovations, Davis, Calif., USA), Rhizobium leguminosarum bv. phaseolii (e.g. RHIZO-STICK from Becker Underwood, USA), R. I. trifolii RP113-7 (e.g. DORMAL from Becker Underwood, USA; Appl. Environ. Microbiol. 44(5), 1096-1101), R. I. bv. viciae P1NP3Cst (also referred to as 1435; New Phytol 179(1), 224-235, 2008; e.g. in NODULATOR PL Peat Granule from Becker Underwood, USA; or in NODULATOR XL PL bfrom Becker Underwood, Canada), R. I. bv. viciae SU303 (e.g. NODULAID Group E from Becker Underwood, Australia), R. I. bv. viciae WSM1455 (e.g. NODULAID Group F from Becker Underwood, Australia), R. tropici SEMIA 4080 (identical to PRF 81; Soil Biology & Biochemistry 39, 867-876, 2007), Sinorhizobium meliloti MSDJ0848 (INRA, France) also referred to as strain 2011 or RCR2011 (Mol Gen Genomics (2004) 272: 1-17; e.g. DORMAL ALFALFA from Becker Underwood, USA; NITRAGIN® Gold from Novozymes Biologicals BioAg Group, Canada), Sphaerodes mycoparasitica IDAC 301008-01 (WO 2011/022809), Steinernema carpocapsae (e.g. MILLENIUM® from Becker Underwood Ltd., UK), S. feltiae (NEMASHIELD® from BioWorks, Inc., USA; NEMASYS® from Becker Underwood Ltd., UK), S. kraussei L137 (NEMASYS® L from Becker Underwood Ltd., UK), Streptomyces griseoviridis K61 (e.g. MYCOSTOP® from Verdera Oy, Espoo, Finland; Crop Protection 25, 468-475, 2006), S. lydicus WYEC 108 (e.g. Actinovate® from Natural Industries, Inc., USA, U.S. Pat. No. 5,403,584), S. violaceusniger YCED-9 (e.g. DT-9® from Natural Industries, Inc., USA, U.S. Pat. No. 5,968,503), Talaromyces flavus V117b (e.g. PROTUS® from Prophyta, Germany), Trichoderma asperellum SKT-1 (e.g. ECO-HOPE® from Kumiai Chemical Industry Co., Ltd., Japan), T. asperellum ICC 012 (e.g. in TENET WP, REMDIER WP, BIOTEN WP from Isagro NC, USA, BIO-TAM from AgraQuest, USA), T. atroviride LC52 (e.g. SENTINEL® from Agrimm Technologies Ltd, NZ), T. atroviride CNCM 1-1237 (e.g. in Esquive WG from Agrauxine S.A., France, e.g. against pruning wound diseases on vine and plant root pathogens), T. fertile JM41 R (NRRL 50759; e.g. RICH PLUS™ from Becker Underwood Bio Ag SA Ltd, South Africa), T. gamsii ICC 080 (e.g. in TENET WP, REMDIER WP, BIOTEN WP from Isagro NC, USA, BIO-TAM from AgraQuest, USA), T. harzianum T-22 (e.g. PLANTSHIELD® der Firma BioWorks Inc., USA), T. harzianum TH 35 (e.g. ROOT PRO® from Mycontrol Ltd., Israel), T. harzianum T-39 (e.g. TRICHODEX® and TRICHODERMA 2000® from Mycontrol Ltd., Israel and Makhteshim Ltd., Israel), T. harzianum and T. viride (e.g. TRICHOPEL from Agrimm Technologies Ltd, NZ), T. harzianum ICC012 and T. viride ICCO80 (e.g. REMEDIER® WP from Isagro Ricerca, Italy), T. polysporum and T. harzianum (e.g. BINAB® from BINAB Bio-lnnovation AB, Sweden), T. stromaticum (e.g. TRICOVAB® from C.E.P.L.A.C., Brazil), T. virens GL-21 (also named Gliocladium virens) (e.g. SOILGARD® from Certis LLC, USA), T. viride (e.g. TRIECO® from Ecosense Labs. (India) Pvt. Ltd., Indien, BIO-CURE® F from T. Stanes & Co. Ltd., Indien), T. viride TV1 (e.g. T. viride TV1 from Agribiotec srl, Italy) and Ulocladium oudemansii HRU3 (e.g. in BOTRY-ZEN® from Botry-Zen Ltd, NZ).

Strains can be sourced from genetic resource and deposition centers: American Type Culture Collection, 10801 University Blvd., Manassas, Va. 20110-2209, USA (strains with ATCC prefic); CABI Europe—International Mycological Institute, Bakeham Lane, Egham, Surrey, TW20 9TYNRRL, UK (strains with prefices CABI and IMI); Centraalbureau voor Schimmelcultures, Fungal Biodiversity Centre, Uppsalaan 8, PO Box 85167, 3508 AD Utrecht, Netherlands (strains with prefic CBS); Division of Plant Industry, CSIRO, Canberra, Australia (strains with prefix CC); Collection Nationale de Cultures de Microorganismes, Institut Pasteur, 25 rue du Docteur Roux, F-75724 PARIS Cedex 15 (strains with prefix CNCM); Leibniz-lnstitut DSMZ-Deutsche Sammlung von Mikroorganismen und Zellkulturen GmbH, InhoffenstraRe 7 B, 38124 Braunschweig, Germany (strains with prefix DSM); International Depositary Authority of Canada Collection, Canada (strains with prefix IDAC); Interntional Collection of Micro-orgniasms from Plants, Landcare Research, Private Bag 92170, Auckland Mail Centre, Auckland 1142, New Zealand (strans with prefix ICMP); IITA, PMB 5320, Ibadan, Nigeria (straisn with prefix IITA); The National Collections of Industrial and Marine Bacteria Ltd., Torry Research Station, P.O. Box 31, 135 Abbey Road, Aberdeen, AB9 8DG, Scotland (strains with prefix NCIMB); ARS Culture Collection of the National Center for Agricultural Utilization Research, Agricultural Research Service, U.S. Department of Agriculture, 1815 North University Street, Peoria, Ill. 61604, USA (strains with prefix NRRL); Department of Scientific and Industrial Research Culture Collection, Applied Biochemistry Division, Palmerston North, New Zealand (strains with prefix NZP); FEPAGRO-Fundação Estadual de Pesquisa Agropecuaria, Rua Gonralves Dias, 570, Bairro Menino Deus, Porto Alegre/RS, Brazil (strains with prefix SEMIA); SARDI, Adelaide, South Australia (strains with prefix SRDI); U.S. Department of Agriculture, Agricultural Research Service, Soybean and Alfalfa Research Laboratory, BARC-West, 10300 Baltimore Boulevard, Building 011, Room 19-9, Beltsville, Md. 20705, USA (strains with prefix USDA: Beltsville Rhizobium Culture Collection Catalog March 1987 USDA-ARS ARS-30: http://pdf.usaid.gov/pdf_docs/PNAAW891.pdf); and Murdoch University, Perth, Western Australia (strains with prefix WSM). Further strains may be found at the Global catalogue of Microorganisms: http://gcm.wfcc.info/ and http://www.landcareresearch.co.nz/resources/collections/icmp and further references to strain collections and their prefixes at http://refs.wdcm.org/collections.htm.

Bacillus amyloliquefaciens subsp. plantarum MBI600 (NRRL B-50595) is deposited under accession number NRRL B-50595 with the strain designation Bacillus subtilis 1430 (and identical to NCIMB 1237). Recently, MBI 600 has been re-classified as Bacillus amyloliquefaciens subsp. plantarum based on polyphasic testing which combines classical microbiological methods relying on a mixture of traditional tools (such as culture-based methods) and molecular tools (such as genotyping and fatty acids analysis). Thus, Bacillus subtilis MBI600 (or MBI 600 or MBI-600) is identical to Bacillus amyloliquefaciens subsp. plantarum MBI600, formerly Bacillus subtilis MBI600. Bacillus amyloliquefaciens MBI600 is known as plant growth-promoting rice seed treatment from Int. J. Microbiol. Res. 3(2) (2011), 120-130 and further described e.g. in US 2012/0149571 A1. This strain MBI600 is e.g. commercially available as liquid formulation product INTEGRAL® (Becker-Underwood Inc., USA).

Bacillus subtilis strain FB17 was originally isolated from red beet roots in North America (System Appl. Microbiol 27 (2004) 372-379). This B. subtilis strain promotes plant health (US 2010/0260735 A1; WO 2011/109395 A2). B. subtilis FB17 has also been deposited at ATCC under number PTA-11857 on Apr. 26, 2011. Bacillus subtilis strain FB17 may be referred elsewhere to as UD1022 or UD10-22.

Bacillus amyloliquefaciens AP-136 (NRRL B-50614), B. amyloliquefaciens AP-188 (NRRL B-50615), B. amyloliquefaciens AP-218 (NRRL B-50618), B. amyloliquefaciens AP-219 (NRRL B-50619), B. amyloliquefaciens AP-295 (NRRL B-50620), B. japonicum SEMIA 5079 (e.g. Gelfix 5 or Adhere 60 from Nitral Urbana Laoboratories, Brazil, a BASF Company), B. japonicum SEMIA 5080 (e.g. GELFIX 5 or ADHERE 60 from Nitral Urbana Laoboratories, Brazil, a BASF Company), B. mojavensis AP-209 (NRRL B-50616), B. solisalsi AP-217 (NRRL B-50617), B. pumilus strain INR-7 (otherwise referred to as BU-F22 (NRRL B-50153) and BU-F33 (NRRL B-50185)), B. simplex ABU 288 (NRRL B-50340) and B. amyloliquefaciens subsp. plantarum MBI600 (NRRL B-50595) have been mentioned i.a. in US patent appl. 20120149571, U.S. Pat. No. 8,445,255, WO 2012/079073. Bradyrhizobium japonicum USDA 3 is known from U.S. Pat. No. 7,262,151.

Jasmonic acid or salts (jasmonates) or derivatives include without limitation potassium jasmonate, sodium jasmonate, lithium jasmonate, ammonium jasmonate, dimethyl-ammonium jasmonate, isopropylammonium jasmonate, diolammonium jasmonate, diethtriethanolammonium jasmonate, jasmonic acid methyl ester, jasmonic acid amide, jasmonic acid methylamide, jasmonic acid-L-amino acid (amide-linked) conjugates (e.g., conjugates with L-isoleucine, L-valine, L-leucine, or L-phenylalanine), 12-oxo-phytodienoic acid, coronatine, coronafacoyl-L-serine, coronafacoyl-L-threonine, methyl esters of 1-oxo-indanoyl-isoleucine, methyl esters of 1-oxo-indanoyl-leucine, coronalon (2-[(6-ethyl-1-oxo-indane-4-carbonyl)-amino]-3-methyl-pentanoic acid methyl ester), linoleic acid or derivatives thereof and cis-jasmone, or combinations of any of the above.

Humates are humic and fulvic acids extracted from a form of lignite coal and clay, known as leonardite. Humic acids are organic acids that occur in humus and other organically derived materials such as peat and certain soft coal. They have been shown to increase fertilizer efficiency in phosphate and micro-nutrient uptake by plants as well as aiding in the development of plant root systems.

According to one embodiment of the inventive mixtures, the at least one pesticide II is selected from the groups L1) to L6):

L1) Microbial pesticides with fungicidal, bactericidal, viricidal and/or plant defense activator activity: Ampelomyces quisqualis M-10 (L.1.1), Aspergillus flavus NRRL 21882 (L1.2), Aureobasidium pullulans DSM 14940 (L1.3), A. pullulans DSM 14941 (L.1.4), Bacillus amyloliquefaciens AP-136 (NRRL B-50614) (L.1.5), B. amyloliquefaciens AP-188 (NRRL B-50615) (L.1.6), B. amyloliquefaciens AP-218 (NRRL B-50618) (L.1.7), B. amyloliquefaciens AP-219 (NRRL B-50619) (L.1.8), B. amyloliquefaciens AP-295 (NRRL B-50620) (L.1.9), B. amyloliquefaciens FZB42 (L.1.10), B. amyloliquefaciens IN937a (L.1.11), B. amyloliquefaciens IT-45 (CNCM 1-3800) (L.1.12), B. amyloliquefaciens subsp. plantarum MBI600 (NRRL B-50595) (L.1.13), B. mojavensis AP-209 (NRRL B-50616) (L.1.15), B. pumilus INR-7 (otherwise referred to as BU-F22 (NRRL B-50153) and BU-F33 (NRRL B-50185)) (L.1.14), B. pumilus KFP9F (L.1.15), B. pumilus QST 2808 (NRRL B-30087) (L.1.16), B. pumilus GHA 181 (L.1.17), B. simplex ABU 288 (NRRL B-50340) (L.1.18), B. solisalsi AP-217 (NRRL B-50617) (L.1.19), B. subtilis CX-9060 (L.1.20), B. subtilis GB03 (L.1.21), B. subtilis GB07 (L.1.22), B. subtilis QST-713 (NRRL B-21661) (L.1.23), B. subtilis var. amyloliquefaciens FZB24 (L.1.24), B. subtilis var. amyloliquefaciens D747 (L.1.25), Candida oleophila 1-82 (L.1.26), C. oleophila O (L.1.27), C. saitoana (L.1.28), Clavibacter michiganensis (bacteriophages) (L.1.29), Coniothyrium minitans CON/M/91-08 (L.1.30), Cryphonectria parasitica (L.1.31), Cryptococcus albidus (L.1.32), Dilophosphora alopecuri (L.1.33), Fusarium oxysporum (L.1.34), Clonostachys rosea f. catenulata J1446 (also named Gliocladium catenulatum) (L.1.35), Gliocladium roseum 321U (L.1.36), Metschnikowia fructicola NRRL Y-30752 (L.1.37), Microdochium dimerum (L.1.38), Microsphaeropsis ochracea P130A (L.1.39), Muscodor albus QST 20799 (L.1.40), Paenibacillus polymyxa PKB1 (ATCC 202127) (L.1.41), Pantoea vagans C9-1 (L.1.42), Phlebiopsis gigantea (L.1.43), Pichia anomala WRL-76 (L.1.44), Pseudozyma flocculosa PF-A22 UL (L.1.45), Pythium oligandrum DV 74 (L.1.46), Sphaerodes mycoparasitica IDAC 301008-01 (L.1.47), Streptomyces griseoviridis K61 (L.1.48), S. lydicus WYEC 108 (L.1.49), S. violaceusniger XL-2 (L.1.50), S. violaceusniger YCED-9 (L.1.51), Talaromyces flavus V117b (L.1.52), Trichoderma asperellum T34 (L.1.53), T. asperellum SKT-1 (L.1.54), T. asperellum ICC 012 (L.1.55), T. atroviride LC52 (L.1.56), T. atroviride CNCM 1-1237 (L.1.57), T. fertile JM41R (L.1.58), T. gamsii ICC 080 (L.1.59), T. harmatum TH 382 (L.1.60), T. harzianum TH-35 (L.1.61), T. harzianum T-22 (L.1.62), T. harzianum T-39 (L.1.63); mixture of T. harzianum ICC012 and T. viride ICC080 (L.1.64); mixture of T. polysporum and T. harzianum (L.1.65); T. stromaticum (L.1.66), T. virens (also named Gliocladium virens) GL-21 (L.1.67), T. virens G41 (L.1.68), T. viride TV1 (L.1.69), Typhula phacorrhiza 94671 (L.1.70), Ulocladium oudemansii HRU3 (L.1.71), Verticillium dahlia (L.1.72), zucchini yellow mosaic virus (avirulent strain) (L.1.73);

L2) Biochemical pesticides with fungicidal, bactericidal, viricidal and/or plant defense activator activity: chitosan (hydrolysate) (L.2.1), harpin protein (L.2.2), laminarin (L.2.3), Menhaden fish oil (L.2.4), natamycin (L.2.5), Plum pox virus coat protein (L.2.6), potassium bicarbonate (L.2.7), Reynoutria sachlinensis extract (L.2.8), salicylic acid (L.2.9), potassium or sodium bicarbonate (L.2.10), tea tree oil (L.2.11);

L3) Microbial pesticides with insecticidal, acaricidal, molluscidal and/or nematicidal activity: Agrobacterium radiobacter K1026 (L.3.1), A radiobacter K84 (L.3.2), Bacillus firmus 1-1582 (L.3.3); B. thuringiensis ssp. aizawai strains ABTS-1857 (L.3.4), SAN 401 I (L.3.5), ABG-6305 (L.3.6) and ABG-6346 (L.3.7); B. t. ssp. israelensis AM65-52 (L.3.8), B. t. ssp. israelensis SUM-6218 (L.3.9), B. t. ssp. galleriae SDS-502 (L.3.10), B. t. ssp. kurstaki EG 2348 (L.3.11), B. t. ssp. kurstaki SB4 (L.3.12), B. t. ssp. kurstaki ABTS-351 (HD-1) (L.3.13), Beauveria bassiana ATCC 74040 (L.3.14), B. bassiana GHA (L.3.15), B. bassiana H123 (L.3.16), B. bassiana DSM 12256 (L.3.17), B. bassiana PPRI 5339 (L.3.18), B. brongniartii (L.3.19), Burkholderia sp. A396 (L.3.20), Chromobacterium subtsugae PRAA4-1 (L.3.21), Cydia pomonella granulosis virus V22 (L.3.22), Cydia pomonella granulosis virus V1 (L.3.23), Isaria fumosorosea Apopka-97 (L.3.24), Lecanicillium longisporum KV42 (L.3.25), L. longisporum KV71 (L.3.26), L. muscarium (formerly Verticillium lecanii) KV01 (L.3.27), Metarhizium anisopliae FI-985 (L.3.28), M. anisopliae Fl-1045 (L.3.29), M. anisopliae F52 (L.3.30), M. anisopliae ICIPE 69 (L.3.31), M. anisopliae var. acridum IMI 330189 (L.3.32); Nomuraea rileyi strains SA86101 (L.3.33), GU87401 (L.3.34), SR86151 (L.3.35), CG128 (L.3.36) and VA9101 (L.3.37); Paecilomyces fumosoroseus FE 9901 (L.3.38), P. lilacinus 251 (L.3.39), P. lilacinus DSM 15169 (L.3.40), P. lilacinus BCP2 (L.3.41), Paenibacillus popilliae Dutky-1940 (NRRL B-2309=ATCC 14706) (L.3.42), P. popilliae KLN 3, P. popilliae Dutky 1 (L.3.43), Pasteuria spp. Ph3 (L.3.44), Pasteuria spp. ATCC PTA-9643 (L.3.45), Pasteuria spp. ATCC SD-5832 (L.3.46), P. nishizawae PN-1 (L.3.46), P. penetrans (L.3.47), P. ramose (L.3.48), P. reneformis Pr-3 (L.3.49), P. thornea (L.3.50), P. usgae (L.3.51), Pseudomonas fluorescens CL 145A (L.3.52), Steinernema carpocapsae (L.3.53), S. feltiae (L.3.54), S. kraussei L137 (L.3.55);

L4) Biochemical pesticides with insecticidal, acaricidal, molluscidal, pheromone and/or nematicidal activity: L-carvone (L.4.1), citral (L.4.2), (E,Z)-7,9-dodecadien-1-yl acetate (L.4.3), ethyl formate (L.4.4), (E,Z)-2,4-ethyl decadienoate (pear ester) (L.4.5), (Z,Z,E)-7,11,13-hexadecatrienal (L.4.6), heptyl butyrate (L.4.7), isopropyl myristate (L.4.8), cis-jasmone (L.4.9), lavanulyl senecioate (L.4.10), 2-methyl 1-butanol (L.4.11), methyl eugenol (L.4.12), methyl jasmonate (L.4.13), (E,Z)-2,13-octadecadien-1-ol (L.4.14), (E,Z)-2,13-octadecadien-1-ol acetate (L.4.15), (E,Z)-3,13-octadecadien-1-ol (L.4.16), R-1-octen-3-ol (L.4.17), pentatermanone (L.4.18), potassium silicate (L.4.19), sorbitol actanoate (L.4.20), (E,Z,Z)-3,8,11-tetradecatrienyl acetate (L.4.21), (Z,E)-9,12-tetradecadien-1-yl acetate (L.4.22), Z-7-tetradecen-2-one (L.4.23), Z-9-tetradecen-1-yl acetate (L.4.24), Z-11-tetradecenal (L.4.25), Z-11-tetradecen-1-ol (L.4.26), Acacia negra extract (L.4.27), extract of grapefruit seeds and pulp (L.4.28), extract of Chenopodium ambrosiodae (L.4.29), Catnip oil (L.4.30), Neem oil (L.4.31), Quillay extract (L.4.32), Tagetes oil (L.4.33);

L5) Microbial pesticides with plant stress reducing, plant growth regulator, plant growth promoting and/or yield enhancing activity: Azospirillum amazonense BR 11140 (SpY2T) (L.5.1), A. brasilense AZ39 (L.5.2), A. brasilense XOH (L.5.3), A. brasilense BR 11005 (Sp245) (L.5.4), A. brasilense BR 11002 (L.5.5), A. lipoferum BR 11646 (Sp31) (L.5.6), A. irakense (L.5.7), A. halopraeferens (L.5.8), Bradyrhizobium sp. PNL01 (L.5.9), B. sp. (Arachis) CB1015 (L.5.10), B. sp. (Arachis) USDA 3446 (L.5.11), B. sp. (Arachis) SEMIA 6144 (L.5.12), B. sp. (Arachis) SEMIA 6462 (L.5.13), B. sp. (Arachis) SEMIA 6464 (L.5.14), B. sp. (Vigna) (L.5.15), B. elkanii SEMIA 587 (L.5.16), B. elkanii SEMIA 5019 (L.5.17), B. elkanii U-1301 (L.5.18), B. elkanii U-1302 (L.5.19), B. elkanii USDA 74 (L.5.20), B. elkanii USDA 76 (L.5.21), B. elkanii USDA 94 (L.5.22), B. elkanii USDA 3254 (L.5.23), B. japonicum 532c (L.5.24), B. japonicum CPAC 15 (L.5.25), B. japonicum E-109 (L.5.26), B. japonicum G49 (L.5.27), B. japonicum TA-11 (L.5.28), B. japonicum USDA 3 (L.5.29), B. japonicum USDA 31 (L.5.30), B. japonicum USDA 76 (L.5.31), B. japonicum USDA 110 (L.5.32), B. japonicum USDA 121 (L.5.33), B. japonicum USDA 123 (L.5.34), B. japonicum USDA 136 (L.5.35), B. japonicum SEMIA 566 (L.5.36), B. japonicum SEMIA 5079 (L.5.37), B. japonicum SEMIA 5080 (L.5.38), B. japonicum WB74 (L.5.39), B. liaoningense (L.5.40), B. lupini LL13 (L.5.41), B. lupini WU425 (L.5.42), B. lupini WSM471 (L.5.43), B. lupini WSM4024 (L.5.44), Glomus intraradices RTI-801 (L.5.45), Mesorhizobium sp. WSM1271 (L.5.46), M. sp. WSM1497 (L.5.47), M. ciceri CC1192 (L.5.48), M. huakii (L.5.49), M. loti CC829 (L.5.50), M. loti SU343 (L.5.51), Paenibacillus alvei NAS6G6 (L.5.52), Penicillium bilaiae (L.5.53), Rhizobium leguminosarum bv. phaseolii (L.5.54), R. I. trifolii RP113-7 (L.5.55), R. I. bv. viciae SU303 (L.5.56), R. I. bv. viciae WSM1455 (L.5.57), R. I. bv. viciae P1NP3Cst (L.5.58) R. tropici SEMIA 4088 (L.5.59), Sinorhizobium meliloti MSDJ0848 (L.5.60);

L6) Biochemical pesticides with plant stress reducing, plant growth regulator and/or plant yield enhancing activity: abscisic acid (L.6.1), aluminium silicate (kaolin) (L.6.2), 3-decen-2-one (L.6.3), formononectin (L.6.4), genistein (L.6.5), hesperetin (L.6.6), homobrassinlide (L.6.7), humates (L.6.8), methyl jasmonate (L.6.9), cis-jasmone (L.6.10), lysophosphatidyl ethanlamine (L.6.11), naringenin (L.6.12), polymeric polyhydroxy acid (L.6.13), salicylic acid (L.6.14), Ascophyllum nodosum (Norwegian kelp, Brown kelp) extract (L.6.15) and Ecklonia maxima (kelp) extract (L.6.16).

The present invention furthermore relates to agrochemical compositions comprising a mixture of a compound I (component 1) and at least one biopesticide selected from the group L) (component 2), in particular at least one further fungicidal biopesticide selected from the groups L1) and L2), as described above, and if desired at least one suitable auxiliary. Preference is also given to mixtures comprising as pesticide II (component 2) a biopesticide from group L1), preferably selected from Bacillus amyloliquefaciens AP-136 (NRRL B-50614 and B-50330), B. amyloliquefaciens AP-188 (NRRL B-50615 and B-50331), B. amylolique-faciens AP-218 (NRRL B-50618), B. amyloliquefaciens AP-219 (NRRL B-50619 and B-50332), B. amyloliquefaciens AP-295 (NRRL B-50620 and B-50333), B. amyloliquefaciens IT-45 (CNCM 1-3800), B. amyloliquefaciens subsp. plantarum MBI600 (NRRL B-50595), B. mojavensis AP-209 (NRRL B-50616), B. pumilus INR-7 (otherwise referred to as BU-F22 (NRRL B-50153) and BU-F33 (NRRL B-50185)), B. pumilus KFP9F, B. pumilus QST 2808 (NRRL B-30087), B. pumilus GHA 181, B. simplex ABU 288 (NRRL B-50340), B. solisalsi AP-217 (NRRL B-50617), B. subtilis CX-9060, B. subtilis GB03, B. subtilis GB07, B. subtilis QST-713 (NRRL B-21661), B. subtilis var. amyloliquefaciens FZB24, B. subtilis var. amyloliquefaciens D747, Paenibacillus alvei NAS6G6, Paenibacillus polymyxa PKB1 (ATCC 202127), Sphaerodes mycoparasitica IDAC 301008-01 and Trichoderma fertile JM41R, even more preferably from Bacillus amyloliquefaciens AP-136 (NRRL B-50614), B. amyloliquefaciens AP-188 (NRRL B-50615), B. amyloliquefaciens AP-218 (NRRL B-50618), B. amyloliquefaciens AP-219 (NRRL B-50619), B. amyloliquefaciens AP-295 (NRRL B-50620), B. amyloliquefaciens IT-45 (CNCM 1-3800), B. mojavensis AP-209 (NRRL B-50616), B. pumilus INR-7 (otherwise referred to as BU-F22 (NRRL B-50153) and BU-F33 (NRRL B-50185)), B. pumilus QST 2808 (NRRL B-30087), B. simplex ABU 288 (NRRL B-50340), B. subtilis QST-713 (NRRL B-21661), B. subtilis MBI600 (NRRL B-50595), Paenibacillus alvei NAS6G6, Sphaerodes mycoparasitica IDAC 301008-01 and Trichoderma fertile JM41R.

According to one embodiment of the inventive mixtures, the at least one pesticide II is Bacillus amyloliquefaciens subsp. plantarum MB1600. These mixtures are particularly suitable in soybean.

According to another embodiment of the inventive mixtures, the at least one pesticide II is B. pumilus strain INR-7. These mixtures are particularly suitable in soybean and corn.

According to a further embodiment, the at least one pesticide II is Bacillus simplex, preferably B. simplex strain ABU 288. These mixtures are particularly suitable in soybean and corn.

According to one embodiment of the inventive mixtures, the at least one pesticide II is selected from Bacillus amyloliquefaciens AP-136, B. amyloliquefaciens AP-188, B. amyloliquefaciens AP-218, B. amyloliquefaciens AP-219, B. amyloliquefaciens AP-295, B. amyloliquefaciens FZB42, B. amyloliquefaciens IN937a, B. amyloliquefaciens IT-45, B. amyloliquefaciens subsp. plantarum MBI600, B. mojavensis AP-209, B. pumilus GB34, B. pumilus INR-7, B. pumilus KFP9F, B. pumilus QST 2808, B. pumilus GHA 181, B. simplex ABU 288, B. solisalsi AP-217, B. subtilis CX-9060, B. subtilis GB03, B. subtilis GB07, B. subtilis QST-713, B. subtilis var. amyloliquefaciens FZB24 and B. subtilis var. amyloliquefaciens D747. These mixtures are particularly suitable in soybean and corn, in particular for seed treatment.

According to a further embodiment, the at least one pesticide II is selected from Streptomyces spp. Preferably from S. griseoviridis, S. lydicus and S. violaceusniger, in particular from strains S. griseoviridis K61, S. lydicus WYEC 108, S. violaceusniger XL-2 and S. violaceusniger YCED-9.

According to a further embodiment, the at least one pesticide II is Sphaerodes mycoparasitica, preferably Sphaerodes mycoparasitica strain I DAC 301008-01 (also referred to as strain SMCD2220-01). These mixtures are particularly suitable in soybean, cereals and corn, in particular corn especially to combat Fusarium head blight.

The present invention also relates to mixtures wherein the at least one pesticide II is selected from the following yests and fungi: Ampelomyces quisqualis, in particular strain AQ 10, Aureobasidium pullulans, in particular blastospores of strain DSM14940 or blastospores of strain DSM 14941 or mixtures thereof; Candida oleophila, in particular strains 1-182 and 0, Coniothyrium minitans, in particular strain CON/M/91-8; Dilophosphora alopecuri which reduces annual ryegrass toxicity (ARGT), a disease of livestock resulting from the ingestion of annual ryegrass seed-heads that have been infected by the toxin producing bacterium Rathayibacter toxicus; Gliocladium catenulatum, in particular strain J 1446; Metschnikovia fructicola, in particular strain NRRL Y-30752, Microsphaeropsis ochracea, in particular strain P130A for control of apple scab; (2.13) Muscodor albus, in particular strain QST 20799, Pichia anomala, in particular strain WRL-076, Pseudozyma flocculosa, in particular strain PF-A22 UL; Pythium oligandrum, in particular strain DV74;

The present invention also relates to mixtures wherein the at least one pesticide II is selected from the fungal genus Trichoderma, preferably from the strains Trichoderma asperellum T34, T. asperellum SKT-1, T. asperellum ICC 012, T. atroviride LC52, T. atroviride CNCM 1-1237, T. fertile JM41 R, T. gamsii ICC 080, T. harmatum TH 382, T. harzianum TH-35, T. harzianum T-22, T. harzianum T-39; mixture of T. harzianum ICC012 and T. viride ICC080; mixture of T. polysporum and T. harzianum; T. stromaticum, T. virens GL-21, T. virens G41 and T. viride TV1; in particular T. fertile JM41R.

The present invention also relates to mixtures wherein the at least one pesticide II is selected from the fungal genus Ulocladium, in particular U. oudemansii HRU3.

Preference is also given to mixtures comprising as pesticide II (component 2) a biopesticide from group L2), preferably selected from chitosan (hydrolysate), methyl-jasmonate, cis-jasmone, laminarin, Reynoutria sachlinensis extract and tea tree oil; even more preferable from methyl jasmonate and laminarin.

Preference is also given to mixtures comprising as pesticide II (component 2) a biopesticide from group L3), preferably selected from Agrobacterium radiobacter K1026, Bacillus firmus I-1582, Bacillus thuringiensis ssp. kurstaki SB4, Beauveria bassiana GHA, B. bassiana H123, B. bassiana DSM 12256, B. bassiana PPRI 5339, Metarhizium anisopliae var. acridum IMI 330189, M. anisopliae FI-985, M. anisopliae FI-1045, M. anisopliae F52, M. anisopliae ICIPE 69, Paecilomyces lilacinus DSM 15169, P. lilacinus BCP2, Paenibacillus popilliae Dutky-1940 (NRRL B-2309=ATCC 14706), P. popilliae KLN 3 and P. popilliae Dutky 1, even more preferably from Bacillus thuringiensis ssp. kurstaki SB4, B. bassiana DSM 12256, B. bassiana PPRI 5339, Metarhizium anisopliae var. acridum IMI 330189, M. anisopliae FI-985, M. anisopliae Fl-1045, Paecilomyces lilacinus DSM 15169, P. lilacinus BCP2, Paenibacillus popilliae Dutky-1940 (NRRL B-2309=ATCC 14706), P. popilliae KLN 3 and P. popilliae Dutky 1.

According to a further embodiment, the at least one pesticide II is Beauveria bassiana, preferably selected from Beauveria bassiana ATCC 74040, B. bassiana GHA, B. bassiana H123, B. bassiana DSM 12256 and B. bassiana PPRI 5339, in particular Beauveria bassiana strain PPRI 5339. These mixtures are particularly suitable for wide range of arthropod pests, such as white flies, thrips, mites, aphids, tingids and all their developmental stages (eggs, immature stages, and adults) infesting numerous crops (vegetables, cucurbits, solanaceous fruits, strawberry, flowers and ornamentals, grapevine, citrus, pome, stone fruits, etc.).

Recent studies have shown that these antagonistic fungal strains can effectively control also nut-weevils, wireworms (Agriotes spp.), and Tephritid flies, such as the Mediterranean fruit fly, Ceratitis capitata, the cherry fruit fly, Rhagoletis cerasi, and the olive fly, Bactrocera oleae. They are also useful in soybean and corn.

According to a further embodiment, the at least one pesticide II is Beauveria brongniartii.

According to a further embodiment, the at least one pesticide II is Metarhizium anisopliae or M. anisopliae var. acridium, preferably selected from M. anisopliae Fl-1045, M. anisopliae F52, M. anisopliae var. acridum strains FI-985 and IMI 330189, in particular strain IMI 330189. These mixtures are particularly suitable for control of arthropod pests in soybean and corn.

According to a further embodiment, the at least one pesticide II is Lecanicillium sp., preferably selected from Lecanicillium longisporum KV42, L. longisporum KV71 and L. muscarium (formerly Verticillium lecanii) KV01.

According to a further embodiment, the at least one pesticide II is Paecilomyces fumosoroseus, preferably strain FE 9901 especially for white fly control.

According to a further embodiment, the at least one pesticide II is selected from Nomuraea rileyi, preferably strains SA86101, GU87401, SR86151, CG128 and VA9101; and P. lilacinus, preferably strains 251, DSM 15169 or BCP2, in particular BCP2, which strains especially control the growth of plant-pathogenic nematodes.

According to a further embodiment, the at least one pesticide II is Bacillus firmus, preferably spores of strain CNCM 1-1582, preferable for seed treatment of soybean and corn against nematodes and insects.

According to a further embodiment, the at least one pesticide II is B. cereus preferably spores of CNCM 1-1562, preferable for seed treatment of soybean and corn against nematodes and insects.

According to a further embodiment, the at least one pesticide II is a mixture of spores of B. firmus and B. cereus, preferably mixtures spores of strains CNCM 1-1582 and CNCM 1-1562, preferable for seed treatment of soybean and corn against nematodes and insects.

According to a further embodiment, the at least one pesticide II is selected from Bacillus thuringiensis, preferably B. thuringiensis ssp. aizawai, in particular B. t. ssp. aizawai strains ABTS-18, SAN 401 I, ABG-6305 and ABG-6346, which are effective against different lepidopteran species including also noctuidae.

According to a further embodiment, the at least one pesticide II is selected from Bacillus t. ssp. israelensis, preferably AM65-52, SAN 402 I and ABG-6164, which are applied against larvae of various dipteran pests, e.g. mosquitoes and nematoceres.

According to a further embodiment, the at least one pesticide II is selected from Bacillus t. ssp. kurstaki preferably from strains EG 2348, SB4 and ABTS-351 (HD-1), in particular B. thuringiensis ssp. kurstaki SB4. These strains are used for control of lepidopteran larvae, but without noctuidae.

According to a further embodiment, the at least one pesticide II is selected from Bacillus thuringiensis subsp. tenebrionis, preferably the strains DSM 2803, NB-125 and NB-176, in particular NB-176, which all protect plants e.g. against leaf beetle larvae.

Preference is also given to mixtures comprising as pesticide II (component 2) a biopesticide from group L4), preferably selected from methyl jasmonate, Acacia negra extract, extract of grapefruit seeds and pulp, Catnip oil, Neem oil, Quillay extract and Tagetes oil, in particular methyl jasmonate or water-based Quillay extract.

Preference is also given to mixtures comprising as pesticide II (component 2) a biopesticide from group L5), preferably selected from Azospirillum amazonense BR 11140 (SpY2T), A. brasilense XOH, A. brasilense BR 11005 (Sp245), A. brasilense BR 11002, A. lipoferum BR 11646 (Sp31), A. irakense, A. halopraeferens, Bacillus amyloliquefaciens AP-136 (NRRL B-50614), Bradyrhizobium sp. (Vigna), B. japonicum USDA 3, B. japonicum USDA 31, B. japonicum USDA 76, B. japonicum USDA 110, B. japonicum USDA 121, Glomus intraradices RTI-801, Paenibacillus alvei NAS6G6, Penicillium bilaiae, Rhizobium leguminosarum bv. phaseolii, R. I. trifolii, R. I. bv. viciae, and Sinorhizobium meliloti, more preferably selected from Azospirillum brasilense BR 11005 (Sp245), Bradyrhizobium sp. (Vigna), B. japonicum USDA 3, B. japonicum USDA 31, B. japonicum USDA 76, B. japonicum USDA 110, B. japonicum USDA 121, Rhizobium leguminosarum bv. phaseolii, R. I. trifolii RP113-7, R. I. bv. viciae SU303, R. I. bv. viciae WSM1455, R. tropici SEMIA 4088 and Sinorhizobium meliloti. According to another embodiment of the inventive mixtures, Bradyrhizobium sp. (meaning any Bradyrhizobium species and/or strain) as pesticide II is Bradyrhizobium japonicum (B. japonicum). These mixtures are particularly suitable in soybean. B. japonicum strains were cultivated using media and fermentation techniques known in the art, e.g. in yeast extract-mannitol broth (YEM) at 27° C. for about 5 days.

The present invention also relates to mixtures, wherein the at least one pesticide II is selected from Bradyrhizobium japonicum (B. japonicum) and further comprises a pesticide III, wherein pesticide III is selected from jasmonic acid or salts or derivatives thereof including cis-jasmone, preferably methyl-jasmonate or cis-jasmone.

References for various B. japonicum strains are given e.g. in U.S. Pat. No. 7,262,151 (B. japonicum strains USDA 110 (=IITA 2121, SEMIA 5032, RCR 3427, ARS 1-110, Nitragin 61A89; isolated from Glycine max in Florida in 1959, Serogroup 110; Appl Environ Microbiol 60, 940-94, 1994), USDA 31 (=Nitragin 61A164; isolated from Glycine max in Wisoconsin in 1941, USA, Serogroup 31), USDA 76 (plant passage of strain USDA 74 which has been isolated from Glycine max in California, USA, in 1956, Serogroup 76), USDA 121 (isolated from Glycine max in Ohio, USA, in 1965), USDA 3 (isolated from Glycine max in Virginia, USA, in 1914, Serogroup 6), USDA 121 (Crop Science 26(5), 911-916, 1986) and USDA 136 (=CB 1809, SEMIA 586, Nitragin 61A136, RCR 3407; isolated from Glycine max in Beltsville, Md. in 1961; Appl Environ Microbiol 60, 940-94, 1994). Further suitable B. japonicum strain G49 (INRA, Angers, France) is described in Fernandez-Flouret, D. & Cleyet-Marel, J. C. (1987) C R Acad Agric Fr 73, 163-171), especially for soybean grown in Europe, in particular in France. Further suitable B. japonicum strain TA-11 (TA11 NOD⁺) (NRRL B-18466) is i.a. described in U.S. Pat. No. 5,021,076; Appl Environ Microbiol (1990) 56, 2399-2403 and commercially available as liquid inoculant for soybean (VAULT® NP, Becker Underwood, USA). Further B. japonicum strains as example for pesticide II are described in US2012/0252672A. Further suitable and especially in Canada commercially available strain 532c (The Nitragin Company, Milwaukee, Wis., USA, field isolate from Wisconsin; Nitragin strain collection No. 61A152; Can J Plant Sci 70 (1990), 661-666) (e.g. in RHIZOFLO, HISTICK, HICOAT Super from Becker Underwood, Canada). Preferably, B. japonicum is selected from strains TA-11 and 532c, more preferably a mixture of B. japonicum strains TA-11 and 532c.

Other suitable and commercially available B. japonicum strains (see e.g. Appl Environ Microbiol 2007, 73(8), 2635) are SEMIA 566 (isolated from North American inoculant in 1966 and used in Brazilian commercial inoculants from 1966 to 1978), SEMIA 586 (=CB 1809; originally isolated in Maryland, USA but received from Austrailia in 1966 and used in Brazilian inoculants in 1977), CPAC 15 (=SEMIA 5079; a natural varaiant of SEMIA 566 used in commercial inoculants since 1992) and CPAC 7 (=SEMIA 5080; a natural variant of SEMIA 586 used in commercial inoculants since 1992). These strains are especially suitable for soybean grown in Australia or South America, in particular in Brazil. In particular, mixtures of B. japonicum SEMIA 5079 and SEMIA 5080 are suitable. Some of the abovementioned strains have been re-classified as a novel species Bradyrhizobium elkanii, e.g. strain USDA 76 (Can. J. Microbiol., 1992, 38, 501-505).

Another suitable and commercially available B. japonicum strain is E-109 (variant of strain USDA 138, see e.g. Eur. J. Soil Biol. 45 (2009) 28-35; Biol Fertil Soils (2011) 47:81-89, deposited at Agriculture Collection Laboratory of the Instituto de Microbiologia y Zoologia Agricola (IMYZA), Instituto Nacional de Tecnologiá Agropecuaria (INTA), Castelar, Argentina). This strain is especially suitable for soybean grown in South America, in particular in Argentina.

Another suitable and commercially available B. japonicum strain are WB74 or WB74-1 (e.g. from Stimuplant CC, South Africa or from SoyGro Bio-Fertilizer Ltd, South Africa). These strains are especially suitable for soybean grown in South America and Africa, in particular in South Africa.

The present invention also relates to mixtures, wherein the at least one pesticide II is selected from Bradyrhizobium elkanii and Bradyrhizobium liaoningense (B. elkanii and B. liaoningense), more preferably from B. elkanii. These mixtures are particularly suitable in soybean. B. elkanii and liaoningense were cultivated using media and fermentation techniques known in the art, e.g. in yeast extract-mannitol broth (YEM) at 27° C. for about 5 days.

The present invention also relates to mixtures wherein the at least one pesticide II is selected from selected from B. elkanii and B. liaoningense and further comprises a pesticide Ill, wherein pesticide III is selected from jasmonic acid or salts or derivatives thereof including cis-jasmone, preferably methyl-jasmonate or cis-jasmone.

Suitable and commercially available B. elkanii strains are SEMIA 587 and SEMIA 5019 (=29W) (see e.g. Appl Environ Microbiol 2007, 73(8), 2635) and USDA 3254 and USDA 76 and USDA 94. Preferably, mixtures of B. elkanii strains SEMIA 587 and SEMIA 5019 are useful (e.g. in Gelfix 5 from Nitral Urbana Laboratories, Brazil, a BASF Company). Further commercially available B. elkanii strains are U-1301 and U-1302 (e.g. product Nitroagin® Optimize from Novozymes Bio As S.A., Brazil or NITRASEC for soybean from LAGE y Cia, Brazil). These strains are especially suitable for soybean grown in Australia or South America, in particular in Brazil.

The present invention also relates to mixtures, wherein pesticide II is selected from Bradyrhizobium sp. (Arachis) (B. sp. Arachis) which shall describe the cowpea miscellany cross-inoculation group which includes inter alia indigenous cowpea bradyrhizobia on cowpea (Vigna unguiculata), siratro (Macroptilium atropurpureum), lima bean (Phaseolus lunatus), and peanut (Arachis hypogaea). This mixture comprising as pesticide II B. sp. Arachis is especially suitable for use in peanut, Cowpea, Mung bean, Moth bean, Dune bean, Rice bean, Snake bean and Creeping vigna, in particular peanut.

The present invention also relates to mixtures wherein the at least one pesticide II is selected from B. sp. (Arachis) and further comprises a pesticide III, wherein pesticide III is selected from jasmonic acid or salts or derivatives thereof including cis-jasmone, preferably methyl-jasmonate or cis-jasmone.

Suitable and commercially available B. sp. (Arachis) strain is CB1015 (=IITA 1006, USDA 3446 presumably originally collected in India; from Australian Inoculants Research Group; see e.g. http://www.qaseeds.com.au/inoculant_applic.php). These strains are especially suitable for peanut grown in Australia, North America or South America, in particular in Brazil. Further suitable strain is Bradyrhizobium sp. PNL01 (Becker Underwood, Bisson and Mason, Apr. 29, 2010, Project report, Worcester Polytechnic Institute, Worcester, Mass., USA: http://www.wpi.edu/Pubs/E-project/Available/E-project-042810-163614/).

Suitable and commercially available Bradyrhizobium sp. (Arachis) strains especially for cowpea and peanut but also for soybean are Bradyrhizobium SEMIA 6144, SEMIA 6462 (=BR 3267) and SEMIA 6464 (=BR 3262; see e.g. FEMS Microbiology Letters (2010) 303(2), 123-131; Revista Brasileira de Ciencia do Solo (2011) 35(3); 739-742, ISSN 0100-0683).

The present invention also relates to mixtures, wherein the at least one pesticide II is selected from Bradyrhizobium sp. (Lupine) (also called B. lupini, B. lupines or Rhizobium lupini). This mixture is especially suitable for use in dry beans and lupins.

The present invention also relates to mixtures wherein the at least one pesticide II is selected from Bradyrhizobium sp. (Lupine) (B. lupini) and further comprises a pesticide III, wherein pesticide III is selected from jasmonic acid or salts or derivatives thereof including cis-jasmone, preferably methyl-jasmonate or cis-jasmone.

Suitable and commercially available B. lupini strain is LL13 (isolated from Lupinus iuteus nodules from French soils; deposited at INRA, Dijon and Angers, France; http://agriculture.gouv.fr/IMG/pdf/ch20060216.pdf). This strain is especially suitable for lupins grown in Australia, North America or Europe, in particular in Europe.

Further suitable and commercially available B. lupini strains WU425 (isolated in Esperance, Western Australia from a non-Australian legume Ornthopus compressus), WSM4024 (isolated from lupins in Australia by CRS during a 2005 survey) and WSM471 (isolated from Ornithopus pinnatus in Oyster Harbour, Western Australia) are described e.g. in Palta J. A. and Berger J. B. (eds), 2008, Proceedings 12^{th} International Lupin Conference, 14-18 Sep. 2008, Fremantle, Western Australia. International Lupin Association, Canterbury, New Zealand, 47-50, ISBN 0-86476-153-8: http://www.lupins.org/pdf/conference/2008/Agronomy %20and %20Production/John %20Howie son %20and %20G %200Hara.pdf; Appl. Environ. Microbiol. 71, 7041-7052, 2005; Australian J. Exp. Agricult. 36(1), 63-70, 1996.

The present invention also relates to mixtures, wherein the at least one pesticide II is selected from Mesorhizobium sp. (meaning any Mesorhizobium species and/or strain), more preferably Mesorhizobium ciceri. These mixtures are particularly suitable in cowpea.

The present invention also relates to mixtures wherein the at least one pesticide II is selected from Mesorhizobium sp. and further comprises a pesticide III, wherein pesticide III is selected from jasmonic acid or salts or derivatives thereof including cis-jasmone, preferably methyl-jasmonate or cis-jasmone.

Suitable and commercially available Mesorhizobium sp. strains are e.g. M. ciceri CC1192 (=UPM 848, CECT 5549; from Horticultural Research Station, Gosford, Australia; collected in Israel from Cicer arietinum nodules; Can J Microbial (2002) 48, 279-284) and Mesorhizobium sp. strains WSM1271 (collected in Sardinia, Italy, from plant host Biserrula pelecinus), WSM 1497 (collected in Mykonos, Greece, from plant host Biserrula pelecinus), M. loti strains CC829 (commerical inoculant for Lotus pedunculatus and L. ulginosus in Australia, isolated from L. ulginosus nodules in USA; NZP 2012), M. loti SU343 (a commercial inoculant for Lotus corniculatus in Australia; isolated from host nodules in USA). For references see e.g. Soil Biol Biochem (2004) 36(8), 1309-1317; Plant and Soil (2011) 348(1-2), 231-243).

Suitable and commercially available M. loti strains are e.g. M. loti CC829 for Lotus pedunculatus.

The present invention also relates to mixtures wherein the at least one pesticide II is selected from Mesorhizobium huakuii, also referred to as Rhizobium huakuii (see e.g. Appl. Environ. Microbiol. 2011, 77(15), 5513-5516). These mixtures are particularly suitable in Astralagus, e.g. Astalagus sinicus (Chinese milkwetch), Thermopsis, e.g. Thermopsis luinoides (Goldenbanner) and alike.

The present invention also relates to mixtures wherein the at least one pesticide II is selected from Mesorhizobium huakuii and further comprises a pesticide III, wherein pesticide III is selected from jasmonic acid or salts or derivatives thereof including cis-jasmone, preferably methyl-jasmonate or cis-jasmone.

Suitable and commercially available M. huakuii strain is HN3015 which was isolated from Astralagus sinicus in a rice-growing field of Southern China (see e.g. World J. Microbiol. Biotechn. (2007) 23(6), 845-851, ISSN 0959-3993).

The present invention also relates to mixtures, wherein the at least one pesticide II is selected from Azospirillum amazonense, A. brasilense, A. lipoferum, A. irakense and A. halopraeferens, more preferably from A. brasilense, in particular selected from A. brasilense strains BR 11005 (Sp245) and AZ39 which are both commercially used in Brazil and are obtainable from EMBRAPA-Agribiologia, Brazil. These mixtures are particularly suitable in soybean.

The present invention also relates to mixtures wherein the at least one pesticide II is selected from A. amazonense, A. brasilense, A. lipoferum, A. irakense and A. halopraeferens, more preferably A. brasilense, and further comprises a pesticide III, wherein pesticide III is selected from jasmonic acid or salts or derivatives thereof including cis-jasmone, preferably methyl-jasmonate or cis-jasmone.

The present invention also relates to mixtures wherein the at least one pesticide II is selected from Rhizobium leguminosarum bv. phaseolii; R. I. trifolii, especially strain RP113-7 thereof, R. I. bv. viciae, in particular strains SU303, WSM1455 and P1NP3Cst thereof; R. tropici, especially strain SEMIA 4088 thereof; and Sinorhizobium meliloti, especially strain MSDJ0848 thereof. Sinorhizobium meliloti is commercially available from Becker Underwood as product Dormal® Alfalfa & Luzerne. Rhizobium leguminosarum bv. phaseoli is commercially available from Becker Underwood as product Rhizo Stick. These strains are particulyrly suitable as inoculants for various legumes such as alfalfa, clover, peas, beans, lentils, soybeans, peanuts and others.

The present invention also relates to mixtures wherein the at least one pesticide II is selected from R. leguminosarum bv. phaseolii, R. I. trifolii, R. I. bv. viciae, R. tropici and Sinorhizobium meliloti, and further comprises a pesticide III, wherein pesticide III is selected from jasmonic acid or salts or derivatives thereof including cis-jasmone, preferably methyl-jasmonate or cis-jasmone.

According to a further embodiment, the at least one pesticide II is selected from Delftia acidovorans, in particular strain RAY209, especially in soybean and canola.

According to a further embodiment, the at least one pesticide II is selected from Lysobacter spp., preferably selected from L. antibioticus, in particular strains 13-1 and HS124, preferably in rice or pepper for control of Phytophthora or bacterial leaf blight. According to a further embodiment, the at least one pesticide II is selected from L. enzymogenes, in particular strain 3.1T8.

According to a further embodiment, the at least one pesticide II is selected from Lysobacter spp., preferably selected from Pseudomonas spp., in particular strain MA 342 and Pseudomonas sp. DSM 13134.

According to a further embodiment, the at least one pesticide II is selected from Penicillium bilaiae.

Preference is also given to mixtures comprising as pesticide II (component 2) a biopesticide from group L6), preferably selected from abscisic acid, aluminium silicate (kaolin), humates, Ascophyllum nodosum (Norwegian kelp, Brown kelp) extract and Ecklonia maxima (kelp) extract.

Preference is also given to mixtures comprising as pesticide II a biopesticide selected from the isoflavones formonennitin, hesperetin and naringenin.

Accordingly, the present invention furthermore relates to compositions comprising one compound I (component 1) and one pesticide II (component 2), which pesticide II is selected from the column “Co. 2” of the lines D-1 to D-829 of Table D.

A further embodiment relates to the compositions D-1 to D-829 listed in Table D, where a row of Table D corresponds in each case to a fungicidal composition comprising as active components one of the in the present specification individualized compounds of formula I (component 1) and the respective pesticide II from groups A) to O) (component 2) stated in the row in question. Preferably, the compositions described comprise the active components in synergistically effective amounts.

TABLE D
Compositions comprising as active components one indiviualized
compound I (I) (in Column Co. 1) and as component 2) (in Column
Co. 2) one pesticide from groups A) to O) [which is coded
e.g. as (A.1.1) for azoxystrobin as defined above]

| Mixt. | Co. 1 | Co. 2 |
|---|---|---|
| D-1 | (I) | (A.1.1) |
| D-2 | (I) | (A.1.2) |
| D-3 | (I) | (A.1.3) |
| D-4 | (I) | (A.1.4) |
| D-5 | (I) | (A.1.5) |
| D-6 | (I) | (A.1.6) |
| D-7 | (I) | (A.1.7) |
| D-8 | (I) | (A.1.8) |
| D-9 | (I) | (A.1.9) |
| D-10 | (I) | (A.1.10) |
| D-11 | (I) | (A.1.11) |
| D-12 | (I) | (A.1.12) |
| D-13 | (I) | (A.1.13) |
| D-14 | (I) | (A.1.14) |
| D-15 | (I) | (A.1.15) |
| D-16 | (I) | (A.1.16) |
| D-17 | (I) | (A.1.17) |
| D-18 | (I) | (A.1.18) |
| D-19 | (I) | (A.1.19) |
| D-20 | (I) | (A.1.20) |
| D-21 | (I) | (A.1.21) |
| D-22 | (I) | (A.1.22) |
| D-23 | (I) | (A.1.23) |
| D-24 | (I) | (A.1.24) |
| D-25 | (I) | (A.1.25) |
| D-26 | (I) | (A.1.26) |
| D-27 | (I) | (A.1.27) |
| D-28 | (I) | (A.1.28) |
| D-29 | (I) | (A.1.29) |
| D-30 | (I) | (A.1.30) |
| D-31 | (I) | (A.1.31) |
| D-32 | (I) | (A.1.32) |
| D-33 | (I) | (A.1.33) |
| D-34 | (I) | (A.1.34) |
| D-35 | (I) | (A.1.35) |
| D-36 | (I) | (A.1.36) |
| D-37 | (I) | (A.1.37) |
| D-38 | (I) | (A.1.38) |
| D-39 | (I) | (A.1.39) |
| D-40 | (I) | (A.1.40) |
| D-41 | (I) | (A.2.1) |
| D-42 | (I) | (A.2.2) |
| D-43 | (I) | (A.2.3) |
| D-44 | (I) | (A.2.4) |
| D-45 | (I) | (A.2.5) |
| D-46 | (I) | (A.2.6) |
| D-47 | (I) | (A.2.7) |
| D-48 | (I) | (A.3.1) |
| D-49 | (I) | (A.3.2) |
| D-50 | (I) | (A.3.3) |
| D-51 | (I) | (A.3.4) |
| D-52 | (I) | (A.3.5) |
| D-53 | (I) | (A.3.6) |
| D-54 | (I) | (A.3.7) |
| D-55 | (I) | (A.3.8) |
| D-56 | (I) | (A.3.9) |
| D-57 | (I) | (A.3.10) |
| D-58 | (I) | (A.3.11) |
| D-59 | (I) | (A.3.12) |
| D-60 | (I) | (A.3.13) |
| D-61 | (I) | (A.3.14) |
| D-62 | (I) | (A.3.15) |
| D-63 | (I) | (A.3.16) |
| D-64 | (I) | (A.3.17) |
| D-65 | (I) | (A.3.18) |
| D-66 | (I) | (A.3.19) |
| D-67 | (I) | (A.3.20) |
| D-68 | (I) | (A.3.21) |
| D-69 | (I) | (A.3.22) |
| D-70 | (I) | (A.3.23) |
| D-71 | (I) | (A.3.24) |
| D-72 | (I) | (A.3.25) |
| D-73 | (I) | (A.3.26) |
| D-74 | (I) | (A.3.27) |
| D-75 | (I) | (A.4.1) |
| D-76 | (I) | (A.4.2) |
| D-77 | (I) | (A.4.3) |
| D-78 | (I) | (A.4.4) |
| D-79 | (I) | (A.4.5) |
| D-80 | (I) | (A.4.6) |
| D-81 | (I) | (A.4.7) |
| D-82 | (I) | (A.4.8) |
| D-83 | (I) | (A.4.9) |
| D-84 | (I) | (A.4.10) |
| D-85 | (I) | (A.4.11) |
| D-86 | (I) | (A.4.12) |
| D-87 | (I) | (B.1.1) |
| D-88 | (I) | (B.1.2) |
| D-89 | (I) | (B.1.3) |
| D-90 | (I) | (B.1.4) |
| D-91 | (I) | (B.1.5) |
| D-92 | (I) | (B.1.6) |
| D-93 | (I) | (B.1.7) |
| D-94 | (I) | (B.1.8) |
| D-95 | (I) | (B.1.9) |
| D-96 | (I) | (B.1.10) |
| D-97 | (I) | (B.1.11) |
| D-98 | (I) | (B.1.12) |
| D-99 | (I) | (B.1.13) |
| D-100 | (I) | (B.1.14) |
| D-101 | (I) | (B.1.15) |
| D-102 | (I) | (B.1.16) |
| D-103 | (I) | (B.1.17) |
| D-104 | (I) | (B.1.18) |
| D-105 | (I) | (B.1.19) |
| D-106 | (I) | (B.1.20) |
| D-107 | (I) | (B.1.21) |
| D-108 | (I) | (B.1.22) |
| D-109 | (I) | (B.1.23) |
| D-110 | (I) | (B.1.24) |
| D-111 | (I) | (B.1.25) |
| D-112 | (I) | (B.1.26) |
| D-113 | (I) | (B.1.27) |
| D-114 | (I) | (B.1.28) |
| D-115 | (I) | (B.1.29) |
| D-116 | (I) | (B.1.30) |
| D-117 | (I) | (B.1.31) |
| D-118 | (I) | (B.1.32) |
| D-119 | (I) | (B.1.33) |
| D-120 | (I) | (B.1.34) |
| D-121 | (I) | (B.1.35) |
| D-122 | (I) | (B.1.36) |
| D-123 | (I) | (B.1.37) |
| D-124 | (I) | (B.1.38) |
| D-125 | (I) | (B.1.39) |
| D-126 | (I) | (B.1.40) |
| D-127 | (I) | (B.1.41) |
| D-128 | (I) | (B.1.42) |
| D-129 | (I) | (B.1.43) |
| D-130 | (I) | (B.1.44) |
| D-131 | (I) | (B.1.45) |
| D-132 | (I) | (B.1.46) |
| D-133 | (I) | (B.1.47) |
| D-134 | (I) | (B.1.48) |
| D-135 | (I) | (B.1.49) |
| D-136 | (I) | (B.1.50) |
| D-137 | (I) | (B.2.1) |
| D-138 | (I) | (B.2.2) |
| D-139 | (I) | (B.2.3) |
| D-140 | (I) | (B.2.4) |
| D-141 | (I) | (B.2.5) |
| D-142 | (I) | (B.2.6) |
| D-143 | (I) | (B.2.7) |
| D-144 | (I) | (B.2.8) |
| D-145 | (I) | (B.3.1) |
| D-146 | (I) | (C.1.1) |
| D-147 | (I) | (C.1.2) |
| D-148 | (I) | (C.1.3) |
| D-149 | (I) | (C.1.4) |
| D-150 | (I) | (C.1.5) |
| D-151 | (I) | (C.1.6) |
| D-152 | (I) | (C.1.7) |
| D-153 | (I) | (C.2.1) |
| D-154 | (I) | (C.2.2) |
| D-155 | (I) | (C.2.3) |
| D-156 | (I) | (C.2.4) |
| D-157 | (I) | (C.2.5) |
| D-158 | (I) | (C.2.6) |
| D-159 | (I) | (C.2.7) |
| D-160 | (I) | (D.1.1) |
| D-161 | (I) | (D.1.2) |
| D-162 | (I) | (D.1.3) |
| D-163 | (I) | (D.1.4) |
| D-164 | (I) | (D.1.5) |
| D-165 | (I) | (D.1.6) |
| D-166 | (I) | (D.2.1) |
| D-167 | (I) | (D.2.2) |
| D-168 | (I) | (D.2.3) |
| D-169 | (I) | (D.2.4) |
| D-170 | (I) | (D.2.5) |
| D-171 | (I) | (D.2.6) |
| D-172 | (I) | (D.2.7) |
| D-173 | (I) | (E.1.1) |
| D-174 | (I) | (E.1.2) |
| D-175 | (I) | (E.1.3) |
| D-176 | (I) | (E.2.1) |
| D-177 | (I) | (E.2.2) |
| D-178 | (I) | (E.2.3) |
| D-179 | (I) | (E.2.4) |
| D-180 | (I) | (E.2.5) |
| D-181 | (I) | (E.2.6) |
| D-182 | (I) | (E.2.7) |
| D-183 | (I) | (E.2.8) |
| D-184 | (I) | (F.1.1) |
| D-185 | (I) | (F.1.2) |
| D-186 | (I) | (F.1.3) |
| D-187 | (I) | (F.1.4) |
| D-188 | (I) | (F.1.5) |
| D-189 | (I) | (F.1.6) |
| D-190 | (I) | (F.2.1) |
| D-191 | (I) | (G.1.1) |
| D-192 | (I) | (G.1.2) |
| D-193 | (I) | (G.1.3) |
| D-194 | (I) | (G.1.4) |
| D-195 | (I) | (G.2.1) |
| D-196 | (I) | (G.2.2) |
| D-197 | (I) | (G.2.3) |
| D-198 | (I) | (G.2.4) |
| D-199 | (I) | (G.2.5) |
| D-200 | (I) | (G.2.6) |
| D-201 | (I) | (G.2.7) |
| D-202 | (I) | (G.3.1) |
| D-203 | (I) | (G.3.2) |
| D-204 | (I) | (G.3.3) |
| D-205 | (I) | (G.3.4) |
| D-206 | (I) | (G.3.5) |
| D-207 | (I) | (G.3.6) |
| D-208 | (I) | (G.3.7) |
| D-209 | (I) | (G.3.8) |
| D-210 | (I) | (G.4.1) |
| D-211 | (I) | (G.5.1) |
| D-212 | (I) | (H.1.1) |
| D-213 | (I) | (H.1.2) |
| D-214 | (I) | (H.1.3) |
| D-215 | (I) | (H.1.4) |
| D-216 | (I) | (H.1.5) |
| D-217 | (I) | (H.1.6) |
| D-218 | (I) | (H.2.1) |
| D-219 | (I) | (H.2.2) |
| D-220 | (I) | (H.2.3) |
| D-221 | (I) | (H.2.4) |
| D-222 | (I) | (H.2.5) |
| D-223 | (I) | (H.2.6) |
| D-224 | (I) | (H.2.7) |
| D-225 | (I) | (H.2.8) |
| D-226 | (I) | (H.2.9) |
| D-227 | (I) | (H.3.1) |
| D-228 | (I) | (H.3.2) |
| D-229 | (I) | (H.3.3) |
| D-230 | (I) | (H.3.4) |
| D-231 | (I) | (H.3.5) |
| D-232 | (I) | (H.3.6) |
| D-233 | (I) | (H.3.7) |
| D-234 | (I) | (H.3.8) |
| D-235 | (I) | (H.3.9) |
| D-236 | (I) | (H.3.10) |
| D-237 | (I) | (H.3.11) |
| D-238 | (I) | (H.4.1) |
| D-239 | (I) | (H.4.2) |
| D-240 | (I) | (H.4.3) |
| D-241 | (I) | (H.4.4) |
| D-242 | (I) | (H.4.5) |
| D-243 | (I) | (H.4.6) |
| D-244 | (I) | (H.4.7) |
| D-245 | (I) | (H.4.8) |
| D-246 | (I) | (H.4.9) |
| D-247 | (I) | (H.4.10) |
| D-248 | (I) | (I.1.1) |
| D-249 | (I) | (I.1.2) |
| D-250 | (I) | (I.2.1) |
| D-251 | (I) | (I.2.2) |
| D-252 | (I) | (I.2.3) |
| D-253 | (I) | (I.2.4) |
| D-254 | (I) | (I.2.5) |
| D-255 | (I) | (J.1.1) |
| D-256 | (I) | (J.1.2) |
| D-257 | (I) | (J.1.3) |
| D-258 | (I) | (J.1.4) |
| D-259 | (I) | (J.1.5) |
| D-260 | (I) | (J.1.6) |
| D-261 | (I) | (J.1.7) |
| D-262 | (I) | (J.1.8) |
| D-263 | (I) | (J.1.9) |
| D-264 | (I) | (K.1.1) |
| D-265 | (I) | (K.1.2) |
| D-266 | (I) | (K.1.3) |
| D-267 | (I) | (K.1.4) |
| D-268 | (I) | (K.1.5) |
| D-269 | (I) | (K.1.6) |
| D-270 | (I) | (K.1.7) |
| D-271 | (I) | (K.1.8) |
| D-272 | (I) | (K.1.9) |
| D-273 | (I) | (K.1.10) |
| D-274 | (I) | (K.1.11) |
| D-275 | (I) | (K.1.12) |
| D-276 | (I) | (K.1.13) |
| D-277 | (I) | (K.1.14) |
| D-278 | (I) | (K.1.15) |
| D-279 | (I) | (K.1.16) |
| D-280 | (I) | (K.1.17) |
| D-281 | (I) | (K.1.18) |
| D-282 | (I) | (K.1.19) |
| D-283 | (I) | (K.1.20) |
| D-284 | (I) | (K.1.21) |
| D-285 | (I) | (K.1.22) |
| D-286 | (I) | (K.1.23) |
| D-287 | (I) | (K.1.24) |
| D-288 | (I) | (K.1.25) |
| D-289 | (I) | (K.1.26) |
| D-290 | (I) | (K.1.27) |
| D-291 | (I) | (K.1.28) |
| D-292 | (I) | (K.1.29) |
| D-293 | (I) | (K.1.30) |
| D-294 | (I) | (K.1.31) |
| D-295 | (I) | (K.1.32) |
| D-296 | (I) | (K.1.33) |
| D-297 | (I) | (K.1.34) |
| D-298 | (I) | (K.1.35) |
| D-299 | (I) | (K.1.36) |
| D-300 | (I) | (K.1.37) |
| D-301 | (I) | (K.1.38) |
| D-302 | (I) | (K.1.39) |
| D-303 | (I) | (K.1.40) |
| D-304 | (I) | (K.1.41) |
| D-305 | (I) | (K.1.42) |
| D-306 | (I) | (K.1.43) |
| D-307 | (I) | (K.1.44) |
| D-308 | (I) | (K.1.45) |
| D-309 | (I) | (K.1.46) |
| D-310 | (I) | (K.1.47) |
| D-311 | (I) | (M.1.1) |
| D-312 | (I) | (M.1.2) |
| D-313 | (I) | (M.1.3) |
| D-314 | (I) | (M.1.4) |
| D-315 | (I) | (M.1.5) |
| D-316 | (I) | (M.1.6) |
| D-317 | (I) | (M.1.7) |
| D-318 | (I) | (M.1.8) |
| D-319 | (I) | (M.1.9) |
| D-320 | (I) | (M.1.10) |
| D-321 | (I) | (M.1.11) |
| D-322 | (I) | (M.1.12) |
| D-323 | (I) | (M.1.13) |
| D-324 | (I) | (M.1.14) |
| D-325 | (I) | (M.1.15) |
| D-326 | (I) | (M.1.16) |
| D-327 | (I) | (M.1.17) |
| D-328 | (I) | (M.1.18) |
| D-329 | (I) | (M.1.19) |
| D-330 | (I) | (M.1.20) |
| D-331 | (I) | (M.1.21) |
| D-332 | (I) | (M.1.22) |
| D-333 | (I) | (M.1.23) |
| D-334 | (I) | (M.1.24) |
| D-335 | (I) | (M.1.25) |
| D-336 | (I) | (M.1.26) |
| D-337 | (I) | (M.1.27) |
| D-338 | (I) | (M.1.28) |
| D-339 | (I) | (M.1.29) |
| D-340 | (I) | (M.1.30) |
| D-341 | (I) | (M.1.31) |
| D-342 | (I) | (M.1.32) |
| D-343 | (I) | (M.1.33) |
| D-344 | (I) | (M.1.34) |
| D-345 | (I) | (M.1.35) |
| D-346 | (I) | (M.1.36) |
| D-347 | (I) | (M.1.37) |
| D-348 | (I) | (M.1.38) |
| D-349 | (I) | (M.1.39) |
| D-350 | (I) | (M.1.40) |
| D-351 | (I) | (M.1.41) |
| D-352 | (I) | (M.1.42) |
| D-353 | (I) | (M.1.43) |
| D-354 | (I) | (M.1.44) |
| D-355 | (I) | (M.1.45) |
| D-356 | (I) | (M.1.46) |
| D-357 | (I) | (M.1.47) |
| D-358 | (I) | (M.1.48) |
| D-359 | (I) | (M.1.49) |
| D-360 | (I) | (M.1.50) |
| D-361 | (I) | (N.1.1) |
| D-362 | (I) | (N.1.2) |
| D-363 | (I) | (N.1.3) |
| D-364 | (I) | (N.1.4) |
| D-365 | (I) | (N.1.5) |
| D-366 | (I) | (N.2.1) |
| D-367 | (I) | (N.2.2) |
| D-368 | (I) | (N.2.3) |
| D-369 | (I) | (N.3.1) |
| D-370 | (I) | (N.3.2) |
| D-371 | (I) | (N.3.3) |
| D-372 | (I) | (N.3.4) |
| D-373 | (I) | (N.4.1) |
| D-374 | (I) | (N.5.1) |
| D-375 | (I) | (N.6.1) |
| D-376 | (I) | (N.6.2) |
| D-377 | (I) | (N.6.3) |
| D-378 | (I) | (N.6.4) |
| D-379 | (I) | (N.6.5) |
| D-380 | (I) | (N.7.1) |
| D-381 | (I) | (N.7.2) |
| D-382 | (I) | (N.7.3) |
| D-383 | (I) | (N.8.1) |
| D-384 | (I) | (N.9.1) |
| D-385 | (I) | (N.10.1) |
| D-386 | (I) | (N.10.2) |
| D-387 | (I) | (N.10.3) |
| D-388 | (I) | (N.10.4) |
| D-389 | (I) | (N.10.5) |
| D-390 | (I) | (N.11.1) |
| D-391 | (I) | (N.12.1) |
| D-392 | (I) | (N.12.2) |
| D-393 | (I) | (N.12.3) |
| D-394 | (I) | (N.12.4) |
| D-395 | (I) | (N.13.1) |
| D-396 | (I) | (N.13.2) |
| D-397 | (I) | (N.13.3) |
| D-398 | (I) | (N.13.4) |
| D-399 | (I) | (N.13.5) |
| D-400 | (I) | (N.13.6) |
| D-401 | (I) | (N.13.7) |
| D-402 | (I) | (N.13.8) |
| D-403 | (I) | (N.13.9) |
| D-404 | (I) | (N.14.1) |
| D-405 | (I) | (N.14.2) |
| D-406 | (I) | (N.15.1) |
| D-407 | (I) | (N.16.1) |
| D-408 | (I) | (N.16.2) |
| D-409 | (I) | (N.17.1) |
| D-410 | (I) | (N.17.2) |
| D-411 | (I) | (N.17.3) |
| D-412 | (I) | (N.17.4) |
| D-413 | (I) | (N.17.5) |
| D-414 | (I) | (N.17.6) |
| D-415 | (I) | (N.17.7) |
| D-416 | (I) | (N.17.8) |
| D-417 | (I) | (N.17.9) |
| D-418 | (I) | (N.17.10) |
| D-419 | (I) | (N.17.11) |
| D-420 | (I) | (N.17.12) |
| D-421 | (I) | (O.1.1) |
| D-422 | (I) | (O.1.2) |
| D-423 | (I) | (O.1.3) |
| D-424 | (I) | (O.1.4) |
| D-425 | (I) | (O.1.5) |
| D-426 | (I) | (O.1.6) |
| D-427 | (I) | (O.1.7) |
| D-428 | (I) | (O.1.8) |
| D-429 | (I) | (O.1.9) |
| D-430 | (I) | (O.1.10) |
| D-431 | (I) | (O.1.11) |
| D-432 | (I) | (O.1.12) |
| D-433 | (I) | (O.1.13) |
| D-434 | (I) | (O.1.14) |
| D-435 | (I) | (O.1.15) |
| D-436 | (I) | (O.1.16) |
| D-437 | (I) | (O.1.17) |
| D-438 | (I) | (O.1.18) |
| D-439 | (I) | (O.1.19) |
| D-440 | (I) | (O.1.20) |
| D-441 | (I) | (O.1.21) |
| D-442 | (I) | (O.1.22) |
| D-443 | (I) | (O.1.23) |
| D-444 | (I) | (O.1.24) |
| D-445 | (I) | (O.1.25) |
| D-446 | (I) | (O.1.26) |
| D-447 | (I) | (O.1.27) |
| D-448 | (I) | (O.1.28) |
| D-449 | (I) | (O.1.29) |
| D-450 | (I) | (O.1.30) |
| D-451 | (I) | (O.1.31) |
| D-452 | (I) | (O.1.32) |
| D-453 | (I) | (O.1.33) |
| D-454 | (I) | (O.1.34) |
| D-455 | (I) | (O.1.35) |
| D-456 | (I) | (O.1.36) |
| D-457 | (I) | (O.1.37) |
| D-458 | (I) | (O.1.38) |
| D-459 | (I) | (O.2.1) |
| D-460 | (I) | (O.2.2) |
| D-461 | (I) | (O.2.3) |
| D-462 | (I) | (O.2.4) |
| D-463 | (I) | (O.2.5) |
| D-464 | (I) | (O.2.6) |
| D-465 | (I) | (O.2.7) |
| D-466 | (I) | (O.2.8) |
| D-467 | (I) | (O.2.9) |
| D-468 | (I) | (O.2.10) |
| D-469 | (I) | (O.2.11) |
| D-470 | (I) | (O.2.12) |
| D-471 | (I) | (O.2.13) |
| D-472 | (I) | (O.2.14) |
| D-473 | (I) | (O.2.15) |
| D-474 | (I) | (O.2.16) |
| D-475 | (I) | (O.3.1) |
| D-476 | (I) | (O.3.2) |
| D-477 | (I) | (O.3.3) |
| D-478 | (I) | (O.3.4) |
| D-479 | (I) | (O.3.5) |
| D-480 | (I) | (O.3.6) |
| D-481 | (I) | (O.3.7) |
| D-482 | (I) | (O.3.8) |
| D-483 | (I) | (O.3.9) |
| D-484 | (I) | (O.3.10) |
| D-485 | (I) | (O.3.11) |
| D-486 | (I) | (O.3.12) |
| D-487 | (I) | (O.3.13) |
| D-488 | (I) | (O.3.14) |
| D-489 | (I) | (O.3.15) |
| D-490 | (I) | (O.3.16) |
| D-491 | (I) | (O.3.17) |
| D-492 | (I) | (O.3.18) |
| D-493 | (I) | (O.3.19) |
| D-494 | (I) | (O.3.20) |
| D-495 | (I) | (O.3.21) |
| D-496 | (I) | (O.3.22) |
| D-497 | (I) | (O.3.23) |
| D-498 | (I) | (O.3.24) |
| D-499 | (I) | (O.3.25) |
| D-500 | (I) | (O.3.26) |
| D-501 | (I) | (O.3.27) |
| D-502 | (I) | (O.4.1) |
| D-503 | (I) | (O.4.2) |
| D-504 | (I) | (O.4.3) |
| D-505 | (I) | (O.4.4) |
| D-506 | (I) | (O.4.5) |
| D-507 | (I) | (O.4.6) |
| D-508 | (I) | (O.4.7) |
| D-509 | (I) | (O.4.8) |
| D-510 | (I) | (O.4.9) |
| D-511 | (I) | (O.4.10) |
| D-512 | (I) | (O.4.11) |
| D-513 | (I) | (O.4.12) |
| D-514 | (I) | (O.4.13) |
| D-515 | (I) | (O.4.14) |
| D-516 | (I) | (O.4.15) |
| D-517 | (I) | (O.4.16) |
| D-518 | (I) | (O.4.17) |
| D-519 | (I) | (O.4.18) |
| D-520 | (I) | (O.4.19) |
| D-521 | (I) | (O.4.20) |
| D-522 | (I) | (O.4.21) |
| D-523 | (I) | (O.4.22) |
| D-524 | (I) | (O.4.23) |
| D-525 | (I) | (O.4.24) |
| D-526 | (I) | (O.5.1) |
| D-527 | (I) | (O.5.2) |
| D-528 | (I) | (O.5.3) |
| D-529 | (I) | (O.5.4) |
| D-530 | (I) | (O.5.5) |
| D-531 | (I) | (O.5.6) |
| D-532 | (I) | (O.5.7) |
| D-533 | (I) | (O.5.8) |
| D-534 | (I) | (O.5.9) |
| D-535 | (I) | (O.6.1) |
| D-536 | (I) | (O.6.2) |
| D-537 | (I) | (O.6.3) |
| D-538 | (I) | (O.6.4) |
| D-539 | (I) | (O.6.5) |
| D-540 | (I) | (O.6.6) |
| D-541 | (I) | (O.6.7) |
| D-542 | (I) | (O.7.1) |
| D-543 | (I) | (O.7.2) |
| D-544 | (I) | (O.7.3) |
| D-545 | (I) | (O.7.4) |
| D-546 | (I) | (O.7.5) |
| D-547 | (I) | (O.7.6) |
| D-548 | (I) | (O.8.1) |
| D-549 | (I) | (O.8.2) |
| D-550 | (I) | (O.8.3) |
| D-551 | (I) | (O.8.4) |
| D-552 | (I) | (O.8.5) |
| D-553 | (I) | (O.9.1) |
| D-554 | (I) | (O.9.2) |
| D-555 | (I) | (O.9.3) |
| D-556 | (I) | (O.10.1) |
| D-557 | (I) | (O.11.1) |
| D-558 | (I) | (O.11.2) |
| D-559 | (I) | (O.11.3) |
| D-560 | (I) | (O.11.4) |
| D-561 | (I) | (O.12.1) |
| D-562 | (I) | (O.13.1) |
| D-563 | (I) | (O.14.1) |
| D-564 | (I) | (O.14.2) |
| D-565 | (I) | (O.15.1) |
| D-566 | (I) | (O.15.2) |
| D-567 | (I) | (O.15.3) |
| D-568 | (I) | (O.15.4) |
| D-569 | (I) | (O.15.5) |
| D-570 | (I) | (O.15.6) |
| D-571 | (I) | (O.15.7) |
| D-572 | (I) | (O.15.8) |
| D-573 | (I) | (O.15.9) |
| D-574 | (I) | (O.15.10) |
| D-575 | (I) | (O.15.11) |
| D-576 | (I) | (O.16.1) |
| D-577 | (I) | (O.16.2) |
| D-578 | (I) | (O.16.3) |
| D-579 | (I) | (O.16.4) |
| D-580 | (I) | (O.16.5) |
| D-581 | (I) | (O.16.6) |
| D-582 | (I) | (L.1.1) |
| D-583 | (I) | (L.1.2) |
| D-584 | (I) | (L.1.3) |
| D-585 | (I) | (L.1.4) |
| D-586 | (I) | (L.1.5) |
| D-587 | (I) | (L.1.6) |
| D-588 | (I) | (L.1.7) |
| D-589 | (I) | (L.1.8) |
| D-590 | (I) | (L.1.9) |
| D-591 | (I) | (L.1.10) |
| D-592 | (I) | (L.1.11) |
| D-593 | (I) | (L.1.12) |
| D-594 | (I) | (L.1.13) |
| D-595 | (I) | (L.1.14) |
| D-596 | (I) | (L.1.15) |
| D-597 | (I) | (L.1.16) |
| D-598 | (I) | (L.1.17) |
| D-599 | (I) | (L.1.18) |
| D-600 | (I) | (L.1.19) |
| D-601 | (I) | (L.1.20) |
| D-602 | (I) | (L.1.21) |
| D-603 | (I) | (L.1.22) |
| D-604 | (I) | (L.1.23) |
| D-605 | (I) | (L.1.24) |
| D-606 | (I) | (L.1.25) |
| D-607 | (I) | (L.1.26) |
| D-608 | (I) | (L.1.27) |
| D-609 | (I) | (L.1.28) |
| D-610 | (I) | (L.1.29) |
| D-611 | (I) | (L.1.30) |
| D-612 | (I) | (L.1.31) |
| D-613 | (I) | (L.1.32) |
| D-614 | (I) | (L.1.33) |
| D-615 | (I) | (L.1.34) |
| D-616 | (I) | (L.1.35) |
| D-617 | (I) | (L.1.36) |
| D-618 | (I) | (L.1.37) |
| D-619 | (I) | (L.1.38) |
| D-620 | (I) | (L.1.39) |
| D-621 | (I) | (L.1.40) |
| D-622 | (I) | (L.1.41) |
| D-623 | (I) | (L.1.42) |
| D-624 | (I) | (L.1.43) |
| D-625 | (I) | (L.1.44) |
| D-626 | (I) | (L.1.45) |
| D-627 | (I) | (L.1.46) |
| D-628 | (I) | (L.1.47) |
| D-629 | (I) | (L.1.48) |
| D-630 | (I) | (L.1.49) |
| D-631 | (I) | (L.1.50) |
| D-632 | (I) | (L.1.51) |
| D-633 | (I) | (L.1.52) |
| D-634 | (I) | (L.1.53) |
| D-635 | (I) | (L.1.54) |
| D-636 | (I) | (L.1.55) |
| D-637 | (I) | (L.1.56) |
| D-638 | (I) | (L.1.57) |
| D-639 | (I) | (L.1.58) |
| D-640 | (I) | (L.1.59) |
| D-641 | (I) | (L.1.60) |
| D-642 | (I) | (L.1.61) |
| D-643 | (I) | (L.1.62) |
| D-644 | (I) | (L.1.63) |
| D-645 | (I) | (L.1.64) |
| D-646 | (I) | (L.1.65) |
| D-647 | (I) | (L.1.66) |
| D-648 | (I) | (L.1.67) |
| D-649 | (I) | (L.1.68) |
| D-650 | (I) | (L.1.69) |
| D-651 | (I) | (L.1.70) |
| D-652 | (I) | (L.1.71) |
| D-653 | (I) | (L.1.72) |
| D-654 | (I) | (L.1.73) |
| D-655 | (I) | (L.2.1) |
| D-656 | (I) | (L.2.2) |
| D-657 | (I) | (L.2.3) |
| D-658 | (I) | (L.2.4) |
| D-659 | (I) | (L.2.5) |
| D-660 | (I) | (L.2.6) |
| D-661 | (I) | (L.2.7) |
| D-662 | (I) | (L.2.8) |
| D-663 | (I) | (L.2.9) |
| D-664 | (I) | (L.2.10) |
| D-665 | (I) | (L.2.11) |
| D-666 | (I) | (L.3.1) |
| D-667 | (I) | (L.3.2) |
| D-668 | (I) | (L.3.3) |
| D-669 | (I) | (L.3.4) |
| D-670 | (I) | (L.3.5) |
| D-671 | (I) | (L.3.6) |
| D-672 | (I) | (L.3.7) |
| D-673 | (I) | (L.3.8) |
| D-674 | (I) | (L.3.9) |
| D-675 | (I) | (L.3.10) |
| D-676 | (I) | (L.3.11) |
| D-677 | (I) | (L.3.12) |
| D-678 | (I) | (L.3.13) |
| D-679 | (I) | (L.3.14) |
| D-680 | (I) | (L.3.15) |
| D-681 | (I) | (L.3.16) |
| D-682 | (I) | (L.3.17) |
| D-683 | (I) | (L.3.18) |
| D-684 | (I) | (L.3.19) |
| D-685 | (I) | (L.3.20) |
| D-686 | (I) | (L.3.21) |
| D-687 | (I) | (L.3.22) |
| D-688 | (I) | (L.3.23) |
| D-689 | (I) | (L.3.24) |
| D-690 | (I) | (L.3.25) |
| D-691 | (I) | (L.3.26) |
| D-692 | (I) | (L.3.27) |
| D-693 | (I) | (L.3.28) |
| D-694 | (I) | (L.3.29) |
| D-695 | (I) | (L.3.30) |
| D-696 | (I) | (L.3.31) |
| D-697 | (I) | (L.3.32) |
| D-698 | (I) | (L.3.33) |
| D-699 | (I) | (L.3.34) |
| D-700 | (I) | (L.3.35) |
| D-701 | (I) | (L.3.36) |
| D-702 | (I) | (L.3.37) |
| D-703 | (I) | (L.3.38) |
| D-704 | (I) | (L.3.39) |
| D-705 | (I) | (L.3.40) |
| D-706 | (I) | (L.3.41) |
| D-707 | (I) | (L.3.42) |
| D-708 | (I) | (L.3.43) |
| D-709 | (I) | (L.3.44) |
| D-710 | (I) | (L.3.45) |
| D-711 | (I) | (L.3.46) |
| D-712 | (I) | (L.3.47) |
| D-713 | (I) | (L.3.48) |
| D-714 | (I) | (L.3.49) |
| D-715 | (I) | (L.3.50) |
| D-716 | (I) | (L.3.51) |
| D-717 | (I) | (L.3.52) |
| D-718 | (I) | (L.3.53) |
| D-719 | (I) | (L.3.54) |
| D-720 | (I) | (L.3.55) |
| D-721 | (I) | (L.4.1) |
| D-722 | (I) | (L.4.2) |
| D-723 | (I) | (L.4.3) |
| D-724 | (I) | (L.4.4) |
| D-725 | (I) | (L.4.5) |
| D-726 | (I) | (L.4.6) |
| D-727 | (I) | (L.4.7) |
| D-728 | (I) | (L.4.8) |
| D-729 | (I) | (L.4.9) |
| D-730 | (I) | (L.4.10) |
| D-731 | (I) | (L.4.11) |
| D-732 | (I) | (L.4.12) |
| D-733 | (I) | (L.4.13) |
| D-734 | (I) | (L.4.14) |
| D-735 | (I) | (L.4.15) |
| D-736 | (I) | (L.4.16) |
| D-737 | (I) | (L.4.17) |
| D-738 | (I) | (L.4.18) |
| D-739 | (I) | (L.4.19) |
| D-740 | (I) | (L.4.20) |
| D-741 | (I) | (L.4.21) |
| D-742 | (I) | (L.4.22) |
| D-743 | (I) | (L.4.23) |
| D-744 | (I) | (L.4.24) |
| D-745 | (I) | (L.4.25) |
| D-746 | (I) | (L.4.26) |
| D-747 | (I) | (L.4.27) |
| D-748 | (I) | (L.4.28) |
| D-749 | (I) | (L.4.29) |
| D-750 | (I) | (L.4.30) |
| D-751 | (I) | (L.4.31) |
| D-752 | (I) | (L.4.32) |
| D-753 | (I) | (L.4.33) |
| D-754 | (I) | (L.5.1) |
| D-755 | (I) | (L.5.2) |
| D-756 | (I) | (L.5.3) |
| D-757 | (I) | (L.5.4) |
| D-758 | (I) | (L.5.5) |
| D-759 | (I) | (L.5.6) |
| D-760 | (I) | (L.5.7) |
| D-761 | (I) | (L.5.8) |
| D-762 | (I) | (L.5.9) |
| D-763 | (I) | (L.5.10) |
| D-764 | (I) | (L.5.11) |
| D-765 | (I) | (L.5.12) |
| D-766 | (I) | (L.5.13) |
| D-767 | (I) | (L.5.14) |
| D-768 | (I) | (L.5.15) |
| D-769 | (I) | (L.5.16) |
| D-770 | (I) | (L.5.17) |
| D-771 | (I) | (L.5.18) |
| D-772 | (I) | (L.5.19) |
| D-773 | (I) | (L.5.20) |
| D-774 | (I) | (L.5.21) |
| D-775 | (I) | (L.5.22) |
| D-776 | (I) | (L.5.23) |
| D-777 | (I) | (L.5.24) |
| D-778 | (I) | (L.5.25) |
| D-779 | (I) | (L.5.26) |
| D-780 | (I) | (L.5.27) |
| D-781 | (I) | (L.5.28) |
| D-782 | (I) | (L.5.29) |
| D-783 | (I) | (L.5.30) |
| D-784 | (I) | (L.5.31) |
| D-785 | (I) | (L.5.32) |
| D-786 | (I) | (L.5.33) |
| D-787 | (I) | (L.5.34) |
| D-788 | (I) | (L.5.35) |
| D-789 | (I) | (L.5.36) |
| D-790 | (I) | (L.5.37) |
| D-791 | (I) | (L.5.38) |
| D-792 | (I) | (L.5.39) |
| D-793 | (I) | (L.5.40) |
| D-794 | (I) | (L.5.41) |
| D-795 | (I) | (L.5.42) |
| D-796 | (I) | (L.5.43) |
| D-797 | (I) | (L.5.44) |
| D-798 | (I) | (L.5.45) |
| D-799 | (I) | (L.5.46) |
| D-800 | (I) | (L.5.47) |
| D-801 | (I) | (L.5.48) |
| D-802 | (I) | (L.5.49) |
| D-803 | (I) | (L.5.50) |
| D-804 | (I) | (L.5.51) |
| D-805 | (I) | (L.5.52) |
| D-806 | (I) | (L.5.53) |
| D-807 | (I) | (L.5.54) |
| D-808 | (I) | (L.5.55) |
| D-809 | (I) | (L.5.56) |
| D-810 | (I) | (L.5.57) |
| D-811 | (I) | (L.5.58) |
| D-812 | (I) | (L.5.59) |
| D-813 | (I) | (L.5.60) |
| D-814 | (I) | (L.6.1) |
| D-815 | (I) | (L.6.2) |
| D-816 | (I) | (L.6.3) |
| D-817 | (I) | (L.6.4) |
| D-818 | (I) | (L.6.5) |
| D-819 | (I) | (L.6.6) |
| D-820 | (I) | (L.6.7) |
| D-821 | (I) | (L.6.8) |
| D-822 | (I) | (L.6.9) |
| D-823 | (I) | (L.6.10) |
| D-824 | (I) | (L.6.11) |
| D-825 | (I) | (L.6.12) |
| D-826 | (I) | (L.6.13) |
| D-827 | (I) | (L.6.14) |
| D-828 | (I) | (L.6.15) |
| D-829 | (I) | (L.6.16) |

The pesticides II and/or III of chemical nature described by their common names, their preparation and their biological activity e.g. against harmful fungi, pests or weed is known (cf.: http://www.alanwood.net/pesticides/); these substances are commercially available and known, for example, from the references below:

benalaxyl, methyl N-(phenylacetyl)-N-(2,6-xylyl)-DL-alaninate (DE 29 03 612); metalaxyl, methyl N-(methoxyacetyl)-N-(2,6-xylyl)-DL-alaninate (GB 15 00 581); ofurace, (RS)-α-(2-chloro-N-2,6-xylylacetamido)-γ-butyrolactone [CAS RN 58810-48-3]; oxadixyl; N-(2,6-dimethylphenyl)-2-methoxy-N-(2-oxo-3-oxazolidinyl)acetamide (GB 20 58 059); aldimorph, “4-alkyl-2,5(or 2,6)-dimethylmorpholine”, comprising 65-75% of 2,6-dimethylmorpholine and 25-35% of 2,5-dimethylmorpholine, comprising more than 85% of 4-dodecyl-2,5(or 2,6)-dimethylmorpholine, where “alkyl” also includes octyl, decyl, tetradecyl and hexadecyl, with a cis/trans ratio of 1:1 [CAS RN 91315-15-0]; dodine,

1-dodecylguanidinium acetate (Plant Dis. Rep., Vol. 41, p.1029 (1957)); dodemorph, 4-cyclododecyl-2,6-dimethylmorpholine (DE 1198125); fenpropimorph, (RS)-cis-4-[3-(4-tert-butylphenyl)-2-methylpropyl]-2,6-dimethylmorpholine (DE 27 52 096); fenpropidin, (RS)-1-[3-(4-tert-butylphenyl)-2-methylpropyl]piperidine (DE 27 52 096); guazatine, mixture of the reaction products from the amidation of technical grade iminodi(octamethylene)diamine, comprising various guanidines and polyamines [CAS RN 108173-90-6]; iminoctadine, 1,1′-iminodi(octamethylene)diguanidine (Congr. Plant Pathol. 1, p.27 (1968); spiroxamine, (8-tert-butyl-1,4-dioxaspiro[4.5]dec-2-yl)diethylamine (EP-A 281 842); tridemorph, 2,6-dimethyl-4-tridecylmorpholine (DE 11 64 152); pyrimethanil, 4,6-dimethylpyrimidin-2-ylphenylamine (DD-A 151 404); mepanipyrim, (4-methyl-6-prop-1-ynylpyrimidin-2-yl)phenylamine (EP-A 224 339); cyprodinil, (4-cyclopropyl-6-methylpyrimidin-2-yl)phenylamine (EP-A 310 550); cycloheximid, 4-{(2R)-2-[(1 S,3S,5S)-3,5-dimethyl-2-oxocyclohexyl]-2-hydroxyethyl}piperidine-2,6-dione [CAS RN 66-81-9]; griseofulvin, 7-chloro-2′,4,6-trimethoxy-6′-methylspiro[benzofuran-2(3H),1′-cyclohex-2′-ene]-3,4′-dione [CAS RN 126-07-8]; kasugamycin, 3-O-[2-amino-4-[(carboxyiminomethyl)amino]-2,3,4,6-tetradeoxy-α-D-arabino-hexopyranosyl]-D-chiro-inositol [CAS RN 6980-18-3]; natamycin, (8E,14E,16E,18E,20E)-(1R,3S,5R,7R,1 2R,22R,24S,25R,26S)-22-(3-amino-3,6-dideoxy-β-D-mannopyranosyloxy)-1,3,26-trihydroxy-12-methyl-10-oxo-6,11,28-trioxatricyclo[22.3.1.05,7]octacosa-8,14,16,18,20-pentaene-25-carboxylic acid [CAS RN 7681-93-8]; polyoxin, 5-(2-amino-5-O-carbamoyl-2-deoxy-L-xylonamido)-1-(5-carboxy-1,2,3,4-tetrahydro-2,4-dioxopyrimidin-1-yl)-1,5-dideoxy-β-D-allofuranuronic acid [CAS RN 22976-86-9]; streptomycin, 1,1′-{1-L-(1,3,5/2,4,6)-4-[5-deoxy-2-O-(2-deoxy-2-methylamino-α-L-glucopyranosyl)-3-C-formyl-α-L-lyxofuranosyloxy]-2,5,6-trihydroxycyclohex-1,3-ylene}diguanidine (J. Am. Chem. Soc. 69, p.1234 (1947)); bitertanol, β-([1,1′-biphenyl]-4-yloxy)-α-(1,1-dimethylethyl)-1H-1,2,4-triazole-1-ethanol (DE 23 24 020); bromuconazole, 1-[[4-bromo-2-(2,4-dichlorophenyl)tetrahydro-2-furanyl]methyl]-1H-1,2,4-triazole (Proc. Br. Crop. Prot. Conf. 1990—Pests Dis. Vol. 1, p. 459); cyproconazole, 2-(4-chlorophenyl)-3-cyclopropyl-1-[1,2,4]triazol-1-ylbutan-2-ol (U.S. Pat. No. 4,664,696); difenoconazole, 1-{2-[2-chloro-4-(4-chlorophenoxy)phenyl]-4-methyl-[1,3]dioxolan-2-ylmethyl}-1H-[1,2,4]triazole (GB-A 2 098 607); diniconazole, (βE)-β-[(2,4-dichlorophenyl)methylene]-α-(1,1-dimethylethyl)-1H-1,2,4-triazole-1-ethanol (Noyaku Kagaku, 1983, Vol. 8, p. 575); enilconazole (imazalil), 1-[2-(2,4-dichlorphenyl)-2-(2-propenyloxy)ethyl]-1H-imidazole (Fruits 28, p. 545, 1973); epoxiconazole, (2RS,3SR)-1-[3-(2-chlorophenyl)-2,3-epoxy-2-(4-fluorophenyl)propyl]-1H-1,2,4-triazole (EP-A 196 038); fenbuconazole, α-[2-(4-chloro-phenyl)ethyl]-α-phenyl-1H-1,2,4-triazole-1-propanenitrile (Proc. Br. Crop Prot. Conf. 1988-Pests Dis. Vol. 1, p. 33); fluquinconazole, 3-(2,4-dichlorophenyl)-6-fluoro-2-[1,2,4]-triazol-1-yl-3H-quinazolin-4-one (Proc. Br. Crop Prot. Conf.-Pests Dis., 5-3, 411 (1992)); flusilazole, 1-{[bis-(4-fluorophenyl)methylsilanyl]methyl}-1H-[1,2,4]triazole (Proc. Br. Crop Prot. Conf.-Pests Dis., 1,413 (1984)); flutriafol, α-(2-fluorophenyl)-α-(4-fluorophenyl)-1H-1,2,4-triazole-1-ethanol (EP 15 756); hexaconazole, 2-(2,4-dichlorophenyl)-1-[1,2,4]triazol-1-ylhexan-2-ol (CAS RN 79983-71-4); ipconazole, 2-[(4-chlorophenyl)methyl]-5-(1-methylethyl)-1-(1H-1,2,4-triazol-1-ylmethyl)cyclopentanol (EP 267 778), metconazole, 5-(4-chlorobenzyl)-2,2-dimethyl-1-[1,2,4]triazol-1-ylmethylcyclopentanol (GB 857 383); myclobutanil, 2-(4-chlorophenyl)-2-[1,2,4]triazol-1-ylmethylpentanenitrile (CAS RN 88671-89-0); penconazole, 1-[2-(2,4-dichlorophenyl)pentyl]-1H-[1,2,4]triazole (Pesticide Manual, 12th Ed. (2000), S.712); propiconazole, 1-[[2-(2,4-dichlorophenyl)-4-propyl-1,3-dioxolan-2-yl]methyl]-1H-1,2,4-triazole (BE 835 579); prochloraz, N-(propyl-[2-(2,4,6-trichlorophenoxy)ethyl])imidazole-1-carboxamide (U.S. Pat. No. 3,991,071); prothioconazole, 2-[2-(1-chlorocyclopropyl)-3-(2-chlorophenyl)-2-hydroxypropyl]-2,4-dihydro-[1,2,4]triazole-3-thione (WO 96/16048); simeconazole, α-(4-fluorophenyl)-α-[(trimethylsilyl)methyl]-1H-1,2,4-triazole-1-ethanol [CAS RN 149508-90-7]; tebuconazole, 1-(4-chlorophenyl)-4,4-dimethyl-3-[1,2,4]triazol-1-ylmethylpentan-3-ol (EP-A 40 345); tetraconazole, 1-[2-(2,4-dichlorophenyl)-3-(1,1,2,2-tetrafluoroethoxy)propyl]-1H-1,2,4-triazole (EP 234 242); triadimefon, 1-(4-chlorophenoxy)-3,3-dimethyl-1-(1H-1,2,4-triazol-1-yl)-2-butanone (BE 793 867); triadimenol, R-(4-chlorophenoxy)-α-(1,1-dimethylethyl)-1H-1,2,4-triazole-1-ethanol (DE 23 24 010); triflumizol, (4-chloro-2-trifluoromethylphenyl)-(2-propoxy-1-[1,2,4]triazol-1-ylethyliden)-amine (JP-A 79/119 462); triticonazole, (5E)-5-[(4-chlorophenyl)methylene]-2,2-dimethyl-1-(1H-1,2,4-triazol-1-ylmethyl)cyclopentanol (FR 26 41 277); iprodione, N-isopropyl-3-(3,5-dichlorophenyl)-2,4-dioxoimidazolidine-1-carboxamide (GB 13 12 536); myclozolin, (RS)-3-(3,5-dichlorophenyl)-5-methoxymethyl-5-methyl-1,3-oxazolidine-2,4-dione [CAS RN 54864-61-8]; procymidone, N-(3,5-dichlorophenyl)-1,2-dimethylcyclopropane-1,2-dicarboximide (U.S. Pat. No. 3,903,090); vinclozolin, 3-(3,5-dichlorophenyl)-5-methyl-5-vinyloxazolidine-2,4-dione (DE-A 22 07 576); ferbam, iron(3+) dimethyldithiocarbamate (U.S. Pat. No. 1,972,961); nabam, disodium ethylenebis(dithiocarbamate) (U.S. Pat. No. 2,317,765); maneb, manganese ethylenebis(dithiocarbamate) (U.S. Pat. No. 2,504,404); mancozeb, manganese ethylenebis-(dithiocarbamate) polymer complex zinc salt (GB 996 264); metam, methyldithiocarbaminic acid (U.S. Pat. No. 2,791,605); metiram, zinc ammoniate ethylenebis(dithiocarbamate) (U.S. Pat. No. 3,248,400); propineb, zinc propylenebis(dithiocarbamate) polymer (BE 611 960); polycarbamate, bis(dimethylcarbamodithioato-S,S′)[p-[[1,2-ethanediylbis[carbamodithioato-S,S′]](2−)]]di[zinc][CAS RN 64440-88-6]; thiram, bis(dimethylthiocarbamoyl) disulfide (DE 642 532); ziram, dimethyldithiocarbamate [CAS RN 137-30-4]; zineb, zinc ethylenebis(dithiocarbamate) (U.S. Pat. No. 2,457,674); anilazine, 4,6-dichloro-N-(2-chlorophenyl)-1,3,5-triazine-2-amine (U.S. Pat. No. 2,720,480); benomyl, N-butyl-2-acetylaminobenzoimidazole-1-carboxamide (U.S. Pat. No. 3,631,176); boscalid, 2-chloro-N-(4′-chlorobiphenyl-2-yl)nicotinamide (EP-A 545 099); carbendazim, methyl (1H-benzoimidazol-2-yl)carbamate (U.S. Pat. No. 3,657,443); carboxin, 5,6-dihydro-2-methyl-N-phenyl-1,4-oxathiin-3-carboxamide (U.S. Pat. No. 3,249,499); oxycarboxin, 5,6-dihydro-2-methyl-1,4-oxathiin-3-carboxanilide 4,4-dioxide (U.S. Pat. No. 3,399,214); cyazofamid, 4-chloro-2-cyano-N,N-dimethyl-5-(4-methylphenyl)-1H-imidazole-1-sulfonamide (CAS RN 120116-88-3]; dazomet, 3,5-dimethyl-1,3,5-thiadiazinane-2-thione (Bull. Soc. Chim. Fr. 15, p. 891 (1897)); dithianon, 5,10-dioxo-5,10-dihydronaphtho[2,3-b][1,4]dithiin-2,3-dicarbonitrile (GB 857 383); famoxadone, (RS)-3-anilino-5-methyl-5-(4-phenoxyphenyl)-1,3-oxazolidine-2,4-dione [CAS RN 131807-57-3]; fenamidone, (S)-1-anilino-4-methyl-2-methylthio-4-phenylimidazolin-5-one [CAS RN 161326-34-7]; fenarimol, α-(2-chlorophenyl)-α-(4-chlorophenyl)-5-pyrimidinemethanol (GB 12 18 623); fuberidazole, 2-(2-furanyl)-1H-benzimidazole (DE 12 09 799); flutolanil, α,α,α-trifluoro-3′-isopropoxy-o-toluanilide (JP 1104514); furametpyr, 5-chloro-N-(1,3-dihydro-1,1,3-trimethyl-4-isobenzofuranyl)-1,3-dimethyl-1H-pyrazole-4-carboxamide [CAS RN 123572-88-3]; isoprothiolane, diisopropyl 1,3-dithiolan-2-ylidenemalonate (Proc. Insectic. Fungic. Conf. 8. Vol. 2, p. 715 (1975)); mepronil, 3′-isopropoxy-o-toluanilide (U.S. Pat. No. 3,937,840); nuarimol, α-(2-chlorophenyl)-α-(4-fluorophenyl)-5-pyrimidinemethanol (GB 12 18 623); fluopicolide (picobenzamid), 2,6-dichloro-N-(3-chloro-5-trifluoromethylpyridin-2-ylmethyl)benzamide (WO 99/42447); probenazole, 3-allyloxy-1,2-benzothiazole 1,1-dioxide (Agric. Biol. Chem. 37, p. 737 (1973)); proquinazid, 6-iodo-2-propoxy-3-propylquinazolin-4(3H)-one (WO 97/48684); pyrifenox, 2′,4′-dichloro-2-(3-pyridyl)acetophenone (EZ)-O-methyloxime (EP 49 854); pyroquilon, 1,2,5,6-tetrahydropyrrolo[3,2,1-ij]quinolin-4-one (GB 139 43 373) quinoxyfen, 5,7-dichloro-4-(4-fluorophenoxy)quinoline (U.S. Pat. No. 5,240,940); silthiofam, N-allyl-4,5-dimethyl-2-(trimethylsilyl)thiophene-3-carboxamide [CAS RN 175217-20-6]; thiabendazole, 2-(1,3-thiazol-4-yl)benzimidazole (U.S. Pat. No. 3,017,415); thifluzamide, 2′,6′-dibromo-2-methyl-4′-tri-fluormethoxy-4-trifluormethyl-1,3-thiazole-5-carboxanilide [CAS RN 130000-40-7]; thiophanate-methyl, 1,2-phenylenebis(iminocarbonothioyl)bis(dimethylcarbamate) (DE-A 19 30 540); tiadinil, 3′-chloro-4,4′-dimethyl-1,2,3-thiadiazole-5-carboxanilide [CAS RN 223580-51-6]; tricyclazole, 5-methyl-1,2,4-triazolo[3,4-b][1,3]benzothiazole [CAS RN 41814-78-2]; triforine, N,N′-{piperazine-1,4-diylbis[(trichlormethyl)methylene]}diformamide (DE 19 01 421); Bordeaux mixture, mixture of CuSO₄×3Cu(OH)₂×3CaSO₄ [CAS RN 8011-63-0]; copper acetate, Cu(OCOCH₃)₂[CAS RN 8011-63-0]; copper oxychloride, Cu₂Cl(OH)₃ [CAS RN 1332-40-7]; basic copper sulfate, CuSO₄ [CAS RN 1344-73-6]; binapacryl, (RS)-2-sec-butyl-4,6-dinitrophenyl 3-methylcrotonate [CAS RN 485-31-4]; dinocap, mixture of 2,6-dinitro-4-octylphenylcrotonate and 2,4-dinitro-6-octylphenylcrotonate, where “octyl” is a mixture of 1-methylheptyl, 1-ethylhexyl and 1-propylpentyl (U.S. Pat. No. 2,526,660); dinobuton, (RS)-2-sec-butyl-4,6-dinitrophenyl isopropyl carbonate [CAS RN 973-21-7]; nitrothal-isopropyl, diisopropyl 5-nitroisophthalate (Proc. Br. Insectic. Fungic. Conf. 7., Vol. 2, p. 673 (1973)); fenpiclonil, 4-(2,3-dichlorophenyl)-1H-pyrrole-3-carbonitrile (Proc. 1988 Br. Crop Prot. Conf.—Pests Dis., Vol. 1, p. 65); fludioxonil, 4-(2,2-difluorobenzo[1,3]dioxol-4-yl)-1H-pyrrole-3-carbonitrile (The Pesticide Manual, publ. The British Crop Protection Council, 10th ed. (1995), p. 482); acibenzolar-S-methyl, methyl 1,2,3-benzothiadiazol-7-carbothioate [CAS RN 135158-54-2]; flubenthiavalicarb (benthiavalicarb), isopropyl {(S)-1-[(1R)-1-(6-fluorobenzothiazol-2-yl)-ethylcarbamoyl]-2-methylpropyl}carbamate (JP-A 09/323 984); carpropamid, 2,2-dichloro-N-[1-(4-chlorophenyl)ethyl]-1-ethyl-3-methylcyclopropanecarboxamide [CAS RN 104030-54-8]; chlorothalonil, 2,4,5,6-tetrachloroisophthalonitrile (U.S. Pat. No. 3,290,353); cyflufenamid, (Z)—N-[α-(cyclopropylmethoxyimino)-2,3-difluoro-6-(trifluoromethyl)benzyl]-2-phenylacetamide (WO 96/19442); cymoxanil, 1-(2-cyano-2-methoxyiminoacetyl)-3-ethylurea (U.S. Pat. No. 3,957,847); diclomezine, 6-(3,5-dichlorophenyl-p-tolyl)pyridazin-3(2H)-one (U.S. Pat. No. 4,052,395) diclocymet, (RS)-2-cyano-N—[(R)-1-(2,4-dichlorophenyl)ethyl]-3,3-dimethylbutyramide [CAS RN 139920-32-4]; diethofencarb, isopropyl 3,4-diethoxycarbanilate (EP 78 663); edifenphos, O-ethyl S,S-diphenyl phosphorodithioate (DE 14 93 736) ethaboxam, N-(cyano-2-thienylmethyl)-4-ethyl-2-(ethylamino)-5-thiazolecarboxamide (EP-A 639 574); fenhexamid, N-(2,3-dichloro-4-hydroxyphenyl)-1-methylcyclohexanecarboxamide (Proc. Br. Crop Prot. Conf.—Pests Dis., 1998, Vol. 2, p. 327); fentin acetate, triphenyltin (U.S. Pat. No. 3,499,086); fenoxanil, N-(1-cyano-1,2-dimethylpropyl)-2-(2,4-dichlorophenoxy)propanamide (EP 262 393); ferimzone, mepanipyrim, (Z)-2′-methylacetophenone-4,6-dimethylpyrimidin-2-ylhydrazone [CAS RN 89269-64-7]; fluazinam, 3-chloro-N-[3-chloro-2,6-dinitro-4-(trifluoromethyl)phenyl]-5-(trifluoromethyl)-2-pyridinamine (The Pesticide Manual, publ. The British Crop Protection Council, 10th ed. (1995), p. 474); fosetyl, fosetyl-aluminum, ethylphosphonate (FR 22 54 276); iprovalicarb, isopropyl [(1S)-2-methyl-1-(1-p-tolylethylcarbamoyl)propyl]carbamate (EP-A 472 996); hexachlorbenzene (C. R. Seances Acad. Agric. Fr. 31, p. 24, 1945); metrafenon, 3′-bromo-2,3,4,6′-tetramethoxy-2′,6-dimethylbenzophenone (U.S. Pat. No. 5,945,567); pencycuron, 1-(4-chlorobenzyl)-1-cyclopentyl-3-phenylurea (DE 27 32 257); penthiopyrad, (RS)—N-[2-(1,3-dimethylbutyl)-3-thienyl]-1-methyl-3-(trifluoromethyl)-1H-pyrazole-4-carboxamide (JP 10130268); propamocarb, propyl 3-(dimethylamino)propylcarbamate (DE 15 67 169); phthalide (DE 16 43 347); toloclofos-methyl, O-2,6-dichloro-p-tolyl O,O-dimethyl phosphorothioate (GB 14 67 561); quintozene, pentachlornitrobenzene (DE 682 048); zoxamide, (RS)-3,5-dichloro-N-(3-chloro-1-ethyl-1-methyl-2-oxopropyl)-p-toluamide [CAS RN 156052-68-5]; azoxystrobin, methyl 2-{2-[6-(2-cyano-1-vinylpenta-1,3-dienyloxy)pyrimidin-4-yloxy]phenyl}-3-methoxyacrylate (EP 382 375), dimoxystrobin, (E)-2-(methoxyimino)-N-methyl-2-[α-(2,5-xylyloxy)-o-tolyl]acetamide (EP 477 631); enestroburin, methyl 2-{2-[3-(4-chlorophenyl)-1-methylallylideneaminooxymethyl]phenyl}-3-methoxyacrylate (EP 936 213); fluoxastrobin, (E)-{2-[6-(2-chlorophenoxy)-5-fluoropyrimidin-4-yloxy]phenyl}(5,6-dihydro-1,4,2-dioxazin-3-yl)methanone O-methyloxime (WO 97/27189); kresoxim-methyl, methyl (E)-methoxyimino[a-(o-tolyloxy)-o-tolyl]acetate (EP 253 213); metominostrobin, (E)-2-(methoxyimino)-N-methyl-2-(2-phenoxyphenyl)acetamide (EP 398 692); orysastrobin, (2E)-2-(methoxyimino)-2-{2-[(3E,5E,6E)-5-(methoxyimino)-4,6-dimethyl-2,8-dioxa-3,7-diazanona-3,6-dien-1-yl]phenyl}-N-methylacetamide (WO 97/15552); picoxystrobin, methyl 3-methoxy-2-[2-(6-trifluoromethylpyridin-2-yloxymethyl)phenyl]acrylate (EP 278 595); pyraclostrobin, methyl N-{2-[1-(4-chlorophenyl)-1H-pyrazol-3-yloxymethyl]phenyl}(N-methoxy)carbamate (WO 96/01256); trifloxystrobin, methyl (E)-methoxyimino-{(E)-α-[1-(α,α,α-trifluoro-m-tolyl)ethylideneaminooxy]-o-tolyl}acetate (EP 460 575); captafol, N-(1,1,2,2-tetrachloroethylthio)cyclohex-4-ene-1,2-dicarboximide (Phytopathology, Vol. 52, p. 754 (1962)); captan, N-(trichloromethylthio)cyclohex-4-ene-1,2-dicarboximide (U.S. Pat. No. 2,553,770); dichlofluanid, N-dichlorofluoromethylthio-N′,N′-dimethyl-N-phenylsulfamide (DE 11 93 498); folpet, N-(trichlormethylthio)phthalimide (U.S. Pat. No. 2,553,770); tolylfluanid, N-dichlorofluoromethyl-thio-N′,N′-dimethyl-N-p-tolylsulfamide (DE 11 93 498); dimethomorph, 3-(4-chlorophenyl)-3-(3,4-dimethoxyphenyl)-1-morpholin-4-yl-propenone (EP 120 321); flumetover, 2-(3,4-dimethoxyphenyl)-N-ethyl-α,α,α-trifluoro-N-methyl-p-toluamide [AGROW no. 243, 22 (1995)]; flumorph, 3-(4-fluorophenyl)-3-(3,4-dimethoxyphenyl)-1-morpholin-4-ylpropenone (EP 860 438); 5-Amino-2-isopropyl-3-oxo-4-o-tolyl-2,3-dihydro-pyrazole-1-carbothioic acid S-allyl ester (CN1939128).

The active substances referred to as component 2 or component 3, their preparation and their activity e.g. against harmful fungi is known (cf.: http://www.alanwood.net/pesticides/); these substances are commercially available. The compounds described by IUPAC nomenclature, their preparation and their fungicidal activity are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 11/028657, WO2012/168188, WO 2007/006670, WO 2011/77514; WO13/047749, WO 10/069882, WO 13/047441, WO 03/16303, WO 09/90181, WO 13/007767, WO 13/010862, PCT/EP2012/065650 and PCT/EP2012/065651).

The mixtures of active substances can be prepared as compositions comprising besides the active ingredients at least one inert ingredient (auxiliary) by usual means, e. g. by the means given for the compositions of compounds I.

Concerning usual ingredients of such compositions reference is made to the explanations given for the compositions containing compounds I.

The mixtures of active substances according to the present invention are suitable as fungicides, as are the compounds of formula I. They are distinguished by an outstanding effectiveness against a broad spectrum of phytopathogenic fungi, especially from the classes of the Ascomycetes, Basidiomycetes, Deuteromycetes and Peronosporomycetes (syn. Oomycetes). In addition, it is referred to the explanations regarding the fungicidal activity of the compounds and the compositions containing compounds I, respectively.

According to one embodiment, the microbial pesticides selected from groups L1), L3) and L5) embrace not only the isolated, pure cultures of the respective micro-organism as defined herein, but also its cell-free extract, its suspensions in a whole broth culture or as a metabolite-containing supernatant or a purified metabolite obtained from a whole broth culture of the microorganism or microorganism strain.

According to a further embodiment, the microbial pesticides selected from groups L1), L3 and L5) embraces not only the isolated, pure cultures of the respective micro-organism as defined herein, but also a cell-free extract thereof or at least one metabolite thereof, and/or a mutant of the respective micro-organism having all the identifying characteristics thereof and also a cell-free extract or at least one metabolite of the mutant.

“Whole broth culture” refers to a liquid culture containing both cells and media.

“Supernatant” refers to the liquid broth remaining when cells grown in broth are removed by centrifugation, filtration, sedimentation, or other means well known in the art.

The term “cell-free extract” refers to an extract of the vegetative cells, spores and/or the whole culture broth of a microorganism comprising cellular metabolites produced by the respective microorganism obtainable by cell disruption methods known in the art such as solvent-based (e.g. organic solvents such as alcohols sometimesin combination with suitable salts), temperature-based, application of shear forces, cell disrupotion with an ultrasonicator.

The desired extract may be concentrated by conventional concentration techniques such as drying, evaporation, centrifugation or alike. Certain washing steps using organic solents and/or water-based media may also be applied to the crude extract preferably prior to use.

The term “metabolite” refers to any compound, substance or byproduct produced by a microorganism (such as fungi and bacteria) that has improves plant growth, water use efficiency of the plant, plant health, plant appearance, or the population of beneficial microorganisms in the soil around the plant activity.

The term “mutant” refers a microorganism obtained by direct mutant selection but also includes microorganisms that have been further mutagenized or otherwise manipulated (e.g., via the introduction of a plasmid). Accordingly, embodiments include mutants, variants, and or derivatives of the respective microorganism, both naturally occurring and artificially induced mutants. For example, mutants may be induced by subjecting the microorganism to known mutagens, such as N-methyl-nitrosoguanidine, using conventional methods. Suitable bactericides are bronopol and isothiazolinone derivatives such as alkyliso-thiazolinones and benzisothiazolinones. Suitable anti-freezing agents are ethylene glycol, propylene glycol, urea and glycerin. Suitable anti-foaming agents are silicones, long chain alcohols, and salts of fatty acids. Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants). Suitable tackifiers or binders are polyvinylpyrrolidons, polyvinylacetates, polyvinyl alcohols, polyacrylates, biological or synthetic waxes, and cellulose ethers.

In the case of mixtures comprising microbial pesticides II selected from groups L1), L3) and L5), the microorganisms as used according to the invention can be cultivated continuously or discontinuously in the batch process or in the fed batch or repeated fed batch process. A review of known methods of cultivation will be found in the textbook by Chmiel (Bioprozesstechnik 1. Einfuhrung in die Bioverfahrenstechnik (Gustav Fischer Verlag, Stuttgart, 1991)) or in the textbook by Storhas (Bioreaktoren und periphere Einrichtungen (Vieweg Verlag, Braunschweig/Wiesbaden, 1994)).

When living microorganisms, such as pesticides II from groups L1), L3) and L5), form part of the compositions, such compositions can be prepared as compositions comprising besides the active ingredients at least one auxiliary (inert ingredient) by usual means (see e.g. H. D. Burges: Formulation of Micobial Biopestcides, Springer, 1998). Suitable customary types of such compositions are suspensions, dusts, powders, pastes, granules, pressings, capsules, and mixtures thereof. Examples for composition types are suspensions (e.g. SC, OD, FS), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). Herein, it has to be taken into account that each formulation type or choice of auxiliary should not influence the viability of the microorganism during storage of the composition and when finally applied to the soil, plant or plant propagation material. Suitable formulations are e.g. mentioned in WO 2008/002371, U.S. Pat. No. 6,955,912, U.S. Pat. No. 5,422,107. Examples for suitable auxiliaries are those mentioned earlier herein, wherein it must be taken care that choice and amounts of such auxiliaries should not influence the viability of the microbial pesticides in the composition. Especially for bactericides and solvents, compatibility with the respective microorganism of the respective microbial pesticide has to be taken into account. In addition, compositions with microbial pesticides may further contain stabilizers or nutrients and UV protectants. Suitable stabilzers or nutrients are e.g. alpha-tocopherol, trehalose, glutamate, potassium sorbate, various sugars like glucose, sucrose, lactose and maltodextrine (H. D. Burges: Formulation of Micobial Biopestcides, Springer, 1998). Suitable UV protectants are e.g. inorganic compounds like titan dioxide, zinc oxide and iron oxide pigments or organic compounds like benzophenones, benzotriazoles and phenyltriazines. The compositions may in addition to auxiliaries mentioned for compositions comprising compounds I herein optionally comprise 0.1-80% stabilizers or nutrients and 0.1-10% UV protectants.

When mixtures comprising microbial pesticides are employed in crop protection, the application rates preferably range from about 1×10⁶ to 5×10¹⁵ (or more) CFU/ha. Preferably, the spore concentration is about 1×10⁷ to about 1×10¹¹ CFU/ha. In the case of (entomopathogenic) nematodes as microbial pesticides (e.g. Steinernema feltiae), the application rates preferably range inform about 1×10⁵ to 1×10¹² (or more), more preferably from 1×10⁸ to 1×10¹¹, even more preferably from 5×10⁸ to 1×10¹⁰ individuals (e.g. in the form of eggs, juvenile or any other live stages, preferably in an infetive juvenile stage) per ha.

When mixtures comprising microbial pesticides are employed in seed treatment, the application rates with respect to plant propagation material preferably range from about 1×10⁶ to 1×10¹² (or more) CFU/seed. Preferably, the concentration is about 1×10⁶ to about 1×10¹¹ CFU/seed. In the case of the microbial pesticides II, the application rates with respect to plant propagation material also preferably range from about 1×10⁷ to 1×10¹⁴ (or more) CFU per 100 kg of seed, preferably from 1×10⁹ to about 1×10¹¹ CFU per 100 kg of seed.

### I. SYNTHESIS EXAMPLES

With appropriate modification of the starting materials, the procedures given in the synthesis examples below were used to obtain further compounds I. The compounds produced in this manner are listed in Table I below including corresponding physical data. 4-Chloro-6-ethyl-5-pyrimidinecarboxylic acid ethyl ester was synthesized according to a procedure given in U.S. Pat. No. 5,439,911 A. 4-Chloro-6-methyl-5-pyrimidinecarboxylic acid methyl ester was prepared as described in EP 606011 A1.

### Example 1

### Preparation of 2-(4-iodophenoxy)-4-(trifluoromethyl)pyridine

To a solution of 4-iodophenol (200 g, 910 mmol) in N,N-dimethylformamide (1 L) was slowly added sodium hydride (47 g, 1.2 mol). The reaction was stirred for 30 min at room temperature, then 2-chloro-4-trifluoromethylpyridine (165 g, 910 mmol) was added and the solution was stirred at 110° C. for 4 h and 12 h at room temperature. The reaction solution was poured into water and extracted with methyl tert-butylether (3×). The combined organic layers were washed successively with water, lithium chloride solution and 10% sodium hydroxide solution. The combined organic phases were then dried over sodium sulfate and the solvent was removed in vacuo to afford 91% (303 g, 830 mmol) yield of 2-(4-iodophenoxy)-4-(trifluoromethyl)pyridine.

### Example 2

### Preparation of 5-[4-[[4-(trifluoromethyl)-2-pyridyl]oxy]phenyl]pent-4-yn-2-amine

To a solution of 2-(4-iodophenoxy)-4-(trifluoromethyl)pyridine (102 g, 279 mmol) in tetrahydrofuran (500 mL) was added triethylamine (84 g, 838 mmol), copper(I) iodide (0.53 g, 3 mmol) and Pd(PPh₃)₂Cl₂ (2 g, 3 mmol), followed by pent-4-yn-2-ol (28 g, 335 mmol). The reaction was stirred at room temperature for 1 h and was then filtered over celite, followed by rinsing with methyl tert-butylether. Water and methyl tert-butylether were then added to the filtrate. The organic layer was separated and concentrated in vacuo to provide 93 g of crude 5-[4-[[4-(trifluoromethyl)-2-pyridyl]oxy]phenyl]pent-4-yn-2-ol. This material was redissolved in dichloromethane (1 L) and triethylamine (58 g, 576 mmol). The reaction solution was cooled to 5° C. and methyanesulfonyl chloride (42 g, 288 mmol) was added. The solution was allowed to warm to room temperature overnight. The reaction solution was poured into water and extracted with dichloromethane. The combined organic layers were dried over sodium sulfate and the solvent was then removed in vacuo to afford 121 g of the crude mesylate. The crude product was redissolved in N,N-dimethylformamide (600 mL) to which sodium azide was added (94 g, 1.4 mol). The reaction solution was heated to 80° C. for 2 h then cooled to room temperature. Water and methyl tert-butylether were added, the organic layers were combined and concentrated in vacuo to provide 91 g of the crude azide product. The azide was dissolved in methanol (700 mL) to which was added tin(II) chloride-H₂O (118 g, 523 mmol). The reaction was stirred at room temperature overnight and was then concentrated. To the residue was added 10% sodium hydroxide solution and the crude product was extracted with dichloromethane before it was dried over sodium sulfate and concentrated in vacuo. The residue was filtered over a silica gel plug to provide 96% of the desired product.

### Example 3

### Preparation of 6-chloro-N5-methyl-N4-[1-methyl-4-[4-[[4-(trifluoromethyl)-2-pyridyl]oxy]phenyl]but-3-ynyl]pyrimidine-4,5-diamine (1-92)

To a solution of 5-[4-[[4-(trifluoromethyl)-2-pyridyl]oxy]phenyl]pent-4-yn-2-amine (401 mg, 1.12 mmool) in N-methyl pyrrolidon (5 mL) was added diisopropylethylamine (1.45 g, 10 mmol). The solution was stirred for 5 min at room temperature at which time 4,6-dichloro-N-methyl-pyrimidin-5-amine (200 mg, 1.12 mmol) was added. The reaction mixture was stirred at 80° C. for 48 h, then allowed to cool to room temperature. The reaction solution was poured into water and extracted with methyl tert-butylether (3×). The combined organic layers were washed with water. The combined organic phases were then dried over sodium sulfate, concentrated, and the residue was purified by flash silica column chromatography (cyclohexane/ethyl acetate) to afford 29% (150 mg, 0.32 mmol) yield the title product as a brown oil.

The compounds listed in Table I have been prepared in an analogous manner.

TABLE I
Compounds I-1 to I-107 of formula I as defined herein and wherein R, R¹ and R² in each
case are hydrogen. Expressions Me and Et refer to groups CH₃ and CH₂CH₃ respectively.

| | | | | | | HPLC Rₜ | m.p. |
|---|---|---|---|---|---|---|---|
| Ex. no | R^{a5} | R^{a6} | Het | R³, R⁴ | (Rb)ₙ* | (min) | (° C.) |
| I-1 | Cl | Cl | H-1 | H, H | n = 0 | 4.327 | 127 |
| I-2 | Cl | Me | H-1 | H, H | n = 0 | 3.196 | 135 |
| I-3 | Cl | Et | H-1 | H, H | n = 0 | 3.301 | 81 |
| I-4 | Cl | Me | H-1 | H, CH₃ | n = 0 | 1.126 | 92 |
| I-5 | Cl | Et | H-1 | H, CH₃ | n = 0 | 1.177 | |
| I-6 | Cl | Cl | H-1 | H, CH₃ | n = 0 | 1.504 | |
| I-7 | #5-CF═CCl—CH═CF-#6 | | H-1 | H, H | n = 0 | 1.263 | 160 |
| I-8 | #5-CF═CH—CH═CF-#6 | | H-1 | H, H | n = 0 | 1.122 | 157 |
| I-9 | #5-CF═CH—CH═CF-#6 | | H-1 | H, CH₃ | n = 0 | 1.181 | 147 |
| I-10 | #5-CF═CF—CH═CF-#6 | | H-1 | H, CH₃ | n = 0 | 1.318 | 144 |
| I-11 | Cl | CHFCH₃ | H-1 | H, H | n = 0 | 1.28 | 79 |
| I-12 | Cl | CHFCH₃ | H-1 | H, CH₃ | n = 0 | 3.915 | |
| I-13 | #5-N(CH₃)—N═CH-#6 | | H-1 | H, CH₃ | n = 0 | 1.07 | 205 |
| I-14 | #5-CH═CH—CH═CF—#6 | | H-1 | H, CH₃ | n = 0 | 1.084 | 126 |
| I-15 | #5-C(CH₃)═CH—CH═CH-#6 | | H-1 | H, CH₃ | n = 0 | 1.107 | |
| I-16 | Me | Cl | H-1 | H, CH₃ | n = 0 | 1.381 | |
| I-18 | #5-N═CH—S-#6 | | H-1 | H, H | n = 0 | 1.239 | 155 |
| I-19 | #5-N═CH—S-#6 | | H-1 | H, CH₃ | n = 0 | 1.299 | |
| I-20 | OMe | H | H-1 | H, CH₃ | n = 0 | 1.034 | |
| I-22 | Cl | COOMe | H-1 | H, CH₃ | n = 0 | 1.36 | 109 |
| I-23 | Cl | CHFCH₃ | H-1 | H, CH₃ | o-F; n | 1.36 | |
| I-24 | Cl | Cl | H-1 | H, CH₃ | o-F; n | 1.48 | |
| I-25 | Cl | CHFCH₃ | H-1 | H, H | o-F; n | 1.29 | |
| I-26 | Cl | Cl | H-1 | H, H | o-F; n | 1.41 | |
| I-28 | #5-N═CH—S-#6 | | H-1 | H, H | o-F; n | 1.25 | 119 |
| I-29 | #5-N(CH₂CH₃)—N═CH-#6 | | H-1 | H, H | n = 0 | 1.036 | |
| I-30 | F | Cl | 5-trifluoromethyl- | H, CH₃ | n = 0 | 1.423 | 112 |
| I-32 | COOEt | Et | H-1 | H, CH₃ | n = 0 | 1.193 | 91 |
| I-33 | #5-N═CH—CH═CH-#6 | | H-1 | H, H | n = 0 | 1.025 | 124 |
| I-34 | #5-N═CH—CH═CH-#6 | | H-1 | H, CH₃ | n = 0 | 1.065 | |
| I-35 | OMe | COOMe | H-1 | H, CH₃ | n = 0 | 1.173 | |
| I-36 | COOMe | Me | H-1 | H, CH₃ | n = 0 | 1.135 | |
| I-37 | Br | Cl | H-1 | H, CH₃ | n = 0 | 1.503 | |
| I-39 | Et | COOMe | H-1 | H, CH₃ | n = 0 | 1.127 | |
| I-40 | Br | F | H-1 | H, CH₃ | n = 0 | 1.473 | |
| I-41 | Br | OH | H-1 | H, CH₃ | n = 0 | 1.245 | |
| I-42 | #5-CF═CH—CH═CF-#6 | | H-4 | H, CH₃ | n = 0 | 1.193 | |
| I-43 | #5-CF═CH—CH═CF-#6 | | H-2 | H, CH₃ | n = 0 | 1.174 | |
| I-44 | #5-CF═CH—CH═CF-#6 | | H-3 | H, CH₃ | n = 0 | 1.186 | 97 |
| I-46 | F | F | H-1 | H, CH₃ | n = 0 | 1.393 | |
| I-47 | Cl | CHFCH₃ | H-2 | H, CH₃ | n = 0 | 1.297 | |
| I-48 | Cl | CHFCH₃ | H-3 | H, CH₃ | n = 0 | 1.329 | |
| I-49 | Cl | CHFCH₃ | H-4 | H, CH₃ | n = 0 | 1.314 | |
| I-50 | Cl | CHFCH₃ | 3,5-dichloro-pyridine-2- | H, CH₃ | n = 0 | 1.39 | |
| I-51 | Cl | Cl | H-2 | H, CH₃ | n = 0 | 1.419 | |
| I-52 | Cl | Cl | H-3 | H, CH₃ | n = 0 | 1.447 | |
| I-53 | Cl | Cl | H-4 | H, CH₃ | n = 0 | 1.434 | |
| I-54 | Cl | Cl | 3,5-dichloro-pyridine-2- | H, CH₃ | n = 0 | 1.519 | |
| I-55 | Me | Cl | H-2 | H, CH₃ | n = 0 | 1.321 | |
| I-56 | Me | Cl | H-3 | H, CH₃ | n = 0 | 1.35 | |
| I-57 | Me | Cl | H-4 | H, CH₃ | n = 0 | 1.344 | |
| I-58 | OMe | Cl | H-2 | H, CH₃ | n = 0 | 1.336 | |
| I-59 | OMe | Cl | H-3 | H, CH₃ | n = 0 | 1.361 | |
| I-60 | OMe | Cl | H-4 | H, CH₃ | n = 0 | 1.358 | |
| I-61 | OMe | Cl | 3,5-dichloro-pyridine-2- | H, CH₃ | n = 0 | 1.434 | |
| I-64 | CN | Cl | H-1 | H, CH₃ | n = 0 | 1.42 | |
| I-65 | Cl | CHFCH₃ | H-16 | H, CH₃ | n = 0 | 1.299 | |
| I-66 | Me | Cl | H-16 | H, CH₃ | n = 0 | 1.324 | |
| I-67 | OMe | Cl | H-16 | H, CH₃ | n = 0 | 1.338 | |
| I-69 | Et | Cl | H-1 | H, CH₃ | n = 0 | 1.43 | |
| I-70 | OMe | CH₂OCH₃ | H-1 | H, CH₃ | n = 0 | 1.07 | |
| I-71 | #5-CF═CH—CH═CF-#6 | | H-16 | H, CH₃ | n = 0 | 1.16 | 97 |
| I-72 | #5-N═N—S-#6 | | H-1 | H, CH₃ | n = 0 | 1.372 | 116 |
| I-73 | CN | Me | H-1 | H, CH₃ | n = 0 | 1.247 | |
| I-74 | #5-CF═CH—CH═CF-#6 | | 3,5-dichloro-pyridine-2- | H, CH₃ | n = 0 | 1.242 | 97 |
| I-75 | Cl | Cl | H-2 | H, CH₃ | n = 0 | 1.427 | |
| I-77 | Cl | CHFCH₃ | H-2 | H, CH₃ | n = 0 | 1.270 | |
| I-78 | Cl | Cl | H-16 | H, CH₃ | n = 0 | 1.428 | |
| I-79 | CH═CH₂ | Cl | H-1 | H, CH₃ | n = 0 | 1.460 | |
| I-80 | Me | Cl | H-30 | H, CH₃ | n = 0 | 1.351 | |
| I-81 | OMe | Cl | H-28 | H, CH₃ | n = 0 | 1.308 | |
| I-82 | Me | COOMe | 4-carboxymethyl-5- | H, CH₃ | n = 0 | 0.976 | 122 |
| | | | methyl-pyrimidine-6-yl | | | | |
| I-83 | F | H | H-1 | H, CH₃ | n = 0 | 1.063 | |
| I-84 | Et | Cl | 4-chloro-5-ethyl- | H, H | n = 0 | 1.405 | |
| | | | pyrimidine-6-yl | | | | |
| I-85 | Cl | Cl | H-36 | H, H | n = 0 | 1.398 | |
| I-86 | #5-N═CH—CH═CH-#6 | | H-23 | H, CH₃ | n = 0 | 0.924 | |
| I-87 | OMe | CH₂OCH₃ | H-1 | H, H | n = 0 | 0.99 | |
| I-88 | #5-N═CH—S-#6 | | H-23 | H, CH₃ | n = 0 | 1.145 | |
| I-89 | Cl | CHFCH₃ | H-23 | H, CH₃ | n = 0 | 1.236 | |
| I-90 | Me | Cl | H-23 | H, CH₃ | n = 0 | 1.229 | |
| I-91 | OMe | Cl | H-23 | H, CH₃ | n = 0 | 1.237 | |
| I-92 | NHMe | Cl | H-1 | H, CH₃ | n = 0 | 1.361 | |
| I-93 | NHEt | Cl | H-1 | H, CH₃ | n = 0 | 1.401 | |
| I-94 | #5-N═CH—CH═N-#6 | | H-1 | H, H | n = 0 | 1.026 | |
| I-95 | #5-N═CH—CH═N-#6 | | H-1 | H, CH₃ | n = 0 | 1.067 | |
| I-96 | H | CN | H-1 | H, CH₃ | n = 0 | 1.02 | |
| I-97 | Cl | CHFCH₃ | H-38 | H, CH₃ | n = 0 | 1.179 | |
| I-98 | Me | Cl | H-38 | H, CH₃ | n = 0 | 1.21 | |
| I-99 | OMe | Cl | H-38 | H, CH₃ | n = 0 | 1.22 | |
| I-100 | NMe₂ | Cl | H-1 | H, CH₃ | n = 0 | 1.435 | |
| I-101 | Cl | Et | H-23 | H, CH₃ | n = 0 | 1.052 | |
| I-102 | OMe | CH₂OCH₃ | H-3 | H, CH₃ | n = 0 | 1.07 | |
| I-103 | Cl | Et | H-3 | H, CH₃ | n = 0 | 1.144 | |
| I-104 | Me | Cl | H-3 | H, CH₃ | n = 0 | 1.097 | |
| I-105 | Cl | Me | H-16 | H, CH₃ | n = 0 | 1.035 | |
| I-106 | OMe | CH₂OCH₃ | H-16 | H, CH₃ | n = 0 | 1.026 | |
| I-107 | Cl | Et | H-16 | H, CH₃ | n = 0 | 1.102 | |

*The position of R^{b} on the phenyl ring is defined relative to the alkyne-moiety bound to the phenyl ring as being in ortho (o-) position; n = 0 indicates that no substituent R^{b} is present on the phenyl ring.
m.p. = melting point (° C.); in cases where R^{a5} and R^{a6} together with two ring member carbon atoms of the pyrimidine ring constitute a fused ring system #5 and #6 indicate the point of attachment to the pyrimidine ring, each respectively corresponding to the positions of either substituent R^{a5} or R^{a6}.
HPLC: HPLC-column Kinetex XB C18 1.7μ (50 × 2.1 mm); eluent: acetonitrile/water + 0.1% TFA (gradient from 5:95 to 100:0 in 1.5 min at 60° C., flow gradient from 0.8 to 1.0 ml/min in 1.5 min).
MS: Quadrupol Electrospray Ionisation, 80 V (positive mode).

### II. BIOLOGICAL EXAMPLES FOR FUNGICIDAL ACTIVITY

The fungicidal action of the compounds I was demonstrated by the following experiments:

A. Glass House Trials

The spray solutions were prepared in several steps: The stock solution were prepared: a mixture of acetone and/or dimethylsulfoxide and the wetting agent/emulsifier Wettol, which is based on ethoxylated alkylphenoles, in a relation (volume) solvent-emulsifier of 99 to 1 was added to 25 mg of the compound to give a total of 5 ml. Water was then added to total volume of 100 ml.

This stock solution was diluted with the described solvent-emulsifier-water mixture to the given concentration.

After the final cultivation period, the extent of fungal attack on the leaves was visually assessed as % diseased leaf area.

### Use Example 1

### Control of Late Blight on Tomatoes Caused by Phytophthora infestans

Young seedlings of tomato plants were grown in pots. These plants were sprayed to run-off with an aqueous suspension, containing the concentration of active ingredient or their mixture mentioned in the table below. The next day, the treated plants were inoculated with an aqueous suspension of sporangia of Phytophthora infestans. After inoculation, the trial plants were immediately transferred to a humid chamber. After 6 days at 18 to 20° C. and a relative humidity close to 100% the extent of fungal attack on the leaves was visually assessed as % diseased leaf area

In this test, the plants which had been treated with 250 ppm of the active compound I-5, I-11, I-14, I-20, I-23, I-34, I-35, I-37, I-44, I-47, I-48, I-50, I-56, I-59, I-65, I-66, I-67, I-70, 1-71, I-79, I-81 or I-82 showed a diseased leaf area of at most 20%, whereas the untreated plants showed 84% diseased leaf area. In this test, the plants which had been treated with 250 ppm of the active 1-84, I-86, I-87, I-88, I-89, I-90, I-91, I-92, I-95, I-97, I-98, I-99, I-101, 1-102, I-103, I-105, I-106 or I-107 showed a diseased leaf area of at most 20%, whereas the untreated plants showed 83% diseased leaf area.

### Use Example 2

### Preventative Control of Leaf Blotch on Wheat Caused by Septoria tritici

Leaves of pot-grown wheat seedling were sprayed to run-off with an aqueous suspension of the active compound or their mixture, prepared as described. The plants were allowed to air-dry. At the following day the plants were inoculated with an aqueous spore suspension of Septoria tritici Then the trial plants were immediately transferred to a humid chamber at 18 to 22° C. and a relative humidity close to 100%. After 4 days the plants were transferred to a chamber with 18 to 22° C. and a relative humidity close to 70%. After 4 weeks the extent of fungal attack on the leaves was visually assessed as % diseased leaf area.

In this test, the plants which had been treated with 250 ppm of the active compound I-1, I-3, I-4, I-5, I-7, I-8, I-9, I-10, I-13, I-14, I-15, I-16, I-18, I-19, I-20, I-22, I-23, I-24, I-25, 1-26, I-28, I-29, I-30, I-32, I-33, I-34, I-35, I-36, I-37, I-40, I-42, I-43, I-44, I-46, I-47, I-48, I-49, 1-50, I-51, I-52, I-53, I-54, I-55, I-56, I-57, I-58, I-59, I-60, I-61, I-64, I-69, I-72, I-74 or I-75 showed a diseased leaf area of at most 20%, whereas the untreated plants showed 92% diseased leaf area.

### Use Example 3

### Preventative Control of Brown Rust on Wheat Caused by Puccinia recondita

The first two developed leaves of pot-grown wheat seedling were sprayed to run-off with an aqueous suspension, containing the concentration of active ingredient or their mixture as described below. The next day the plants were inoculated with spores of Puccinia recondita. To ensure the success the artificial inoculation, the plants were transferred to a humid chamber without light and a relative humidity of 95 to 99% and 20 to 24° C. for 24 h. Then the trial plants were cultivated for 6 days in a greenhouse chamber at 20 to 24° C. and a relative humidity between 65 and 70%. The extent of fungal attack on the leaves was visually assessed as % diseased leaf area.

In this test, the plants which had been treated with 250 ppm of the active compound I-1, I-3, I-4, I-5, I-6, I-8, I-9, I-11, I-12, I-13, I-14, I-15, I-19, I-20, I-23, I-24, I-25, I-26, I-28, 1-29, I-34, I-46, I-47, I-48, I-49, I-50, I-51, I-52, I-54, I-56, I-57, I-58, I-59, I-60, I-64, I-69 or I-73 showed a diseased leaf area of at most 20%, whereas the untreated plants showed 86% diseased leaf area.

### Use Example 4

### Protective Control of Soy Bean Rust on Soy Beans Caused by Phakopsora pachyrhizi

Leaves of pot-grown soy bean seedlings were sprayed to run-off with an aqueous suspension, containing the concentration of active ingredient or their mixture as described below. The plants were allowed to air-dry. The trial plants were cultivated for 1 day in a greenhouse chamber at 23 to 27° C. and a relative humidity between 60 and 80%.Then the plants were inoculated with spores of Phakopsora pachyrhizi. To ensure the success the artificial inoculation, the plants were transferred to a humid chamber with a relative humidity of about 95% and 20 to 24° C. for 24 h. The trial plants were cultivated for fourteen days in a greenhouse chamber at 23 to 27° C. and a relative humidity between 60 and 80%. The extent of fungal attack on the leaves was visually assessed as % diseased leaf area.

In this test, the plants which had been treated with 250 ppm of the active compound I-2, I-4, I-9, I-10, I-18, I-20, I-22, I-29, I-30, I-33, I-36, I-37, I-39, I-42, I-43, I-46, I-48, I-49, 1-50, I-51, I-52, I-55, I-56, I-58, I-59, I-64, I-65, I-66, I-67, I-69, I-71, I-73, I-77, I-80 or I-82 showed a diseased leaf area of at most 15%, whereas the untreated plants showed 94% diseased leaf area. In this test, the plants which had been treated with 250 ppm of the active compound I-83, I-86, I-90, I-92, I-93, I-94, I-95, I-96, I-97, I-101 or I-105 showed a diseased leaf area of at most 15%, whereas the untreated plants showed 95% diseased leaf area.

### Use Example 5

### Curative Action Against Phakopsora pachyrhizion Soybeans (Rust of Soybean)

Leaves of potted soybean seedlings were dusted with a suspension of spores of rust of soybean (Phakopsora pachyrhizi). The plants were then placed in a chamber with high atmospheric humidity (90 to 95%), at 23 to 27° C., for 4 days. During this time, the spores germinated and the germinal tubes penetrated into the leaf tissue. The next day, the infected plants were sprayed to runoff point with an aqueous suspension having the concentration of active substance stated in table 4 (250 ppm). After drying of the sprayed suspension, the test plants were returned to the greenhouse and cultivated at temperatures between 23 and 27° C. and at 60 to 80% relative atmospheric humidity for a further 14 days. The extent of the rust development on the leaves was then determined visually.

In this test, the plants which had been treated with 250 ppm of the active compound 1-87, 1-89,

I-102 or I-106 showed a diseased leaf area of at most 15%, whereas the untreated plants showed 95% diseased leaf area.

### Use Example 6

### Preventative Fungicidal Control of Botrytis cinerea on Leaves of Green Pepper

Young seedlings of green pepper were grown in pots to the 4 to 5 leaf stage. These plants were sprayed to run-off with an aqueous suspension, containing the concentration of active ingredient or their mixture mentioned in the table below. The next day the plants were inoculated with a aqueous biomalt solution containing the spore suspension of Botrytis cinerea. Then the plants were immediately transferred to a humid chamber and kept for 5 days at 22 to 24° C. and a relative humidity close to 100%.

In this test, the plants which had been treated with 250 ppm of the active compound I-2, I-18, 1-22, I-29, I-33, I-35, I-36, I-39, I-41, I-42, I-43, I-60 or I-74 showed a diseased leaf area of at most 15%, whereas the untreated plants showed 95% diseased leaf area.

### Use Example 7

### Control of Late Blight on Tomatoes Caused by Phytophthora infestans

Young seedlings of tomato plants were grown in pots. These plants were sprayed to run-off with an aqueous suspension, containing the concentration of active ingredient or their mixture mentioned in the table below. After seven days the treated plants were inoculated with an aqueous suspension of sporangia of Phytophthora infestans. After inoculation, the trial plants were immediately transferred to a humid chamber and kept for 6 days at 18 to 20° C. and a relative humidity close to 100%.

In this test, the plants which had been treated with 250 ppm of the active compound I-29, I-35, I-37, I-44, I-47, I-48, I-49, I-50, I-56, I-59, I-65, I-66, I-67, I-70 or I-78 showed a diseased leaf area of at most 20%, whereas the untreated plants showed 67% diseased leaf area. In this test, the plants which had been treated with 250 ppm of the active compound I-85, I-89, 1-91, I-100, I-101, I-102, I-103, I-104, I-105, I-106 or I-107 showed a diseased leaf area of at most 20%, whereas the untreated plants showed 84% diseased leaf area.

1-13. (canceled)

14: A compound of formula I 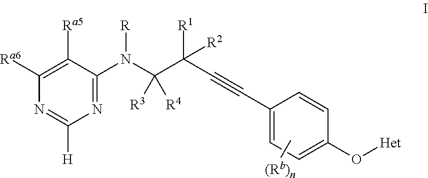
wherein:
R^{a5}, R^{a6} independently of each other are hydrogen, halogen, CN, NO₂, OH, SH, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₁-C₄-alkoxy-C₁-C₄-alkyl, C₁-C₄-alkoxy-C₁-C₄-alkoxy, C₂-C₄-alkenyl, C₂-C₄-alkynyl, C₂-C₄-haloalkenyl, C₂-C₄-haloalkynyl, C₃-C₈-cycloalkyl, C₃-C₈-cycloalkyloxy, C₃-C₈-cycloalkyl-C₁-C₄-alkyl, NR^{A}R^{B}, C(═O)R′, C(═NOR″)R′″ or —C(═NH)—O—R′″;
R^{A}, R^{B} independently of one another are hydrogen, C₁-C₄-alkyl, C₂-C₄-alkenyl, C₂-C₄-alkynyl, phenyl, benzyl, C₃-C₈-cycloalkyl, C₃-C₈-cycloalkenyl or —(C═O)—R′;
R′ is hydrogen, OH, NH₂, C₁-C₄-alkyl, C₁-C₄-haloalkyl, C₂-C₄-alkenyl, C₂-C₄-alkynyl, C₁-C₄-alkoxy, C₁-C₄-haloalkoxy, C₁-C₄-alkylamino or di(C₁-C₄-alkyl)amino;
R″ is hydrogen, C₁-C₄-alkyl, C₁-C₄-haloalkyl, C₂-C₄-alkenyl, C₂-C₄-alkynyl or C₁-C₄-alkoxy-C₁-C₄-alkyl;
R′″ is hydrogen or C₁-C₄-alkyl; or
R^{a5}, R^{a6} together with two ring member carbon atoms to which they are attached, form a fused 5-, 6- or 7-membered saturated, partially unsaturated or aromatic carbocycle or heterocycle, wherein the ring member atoms of the fused heterocycle include besides carbon atoms 1, 2, 3 or 4 heteroatoms selected from N, O and S, and wherein the fused carbocycle or heterocycle is unsubstituted or carries 1, 2, 3 or 4 identical or different radicals selected from halogen, CN, C₁-C₄-alkyl, C₁-C₄-alkoxy, C₁-C₄-haloalkyl and C₁-C₄-haloalkoxy;
R is hydrogen, C₁-C₄-alkyl, C₁-C₄-haloalkyl, C₁-C₄-alkoxy, C₁-C₄-haloalkoxy, C₁-C₄-alkoxy-C₁-C₄-alkyl, C₁-C₄-haloalkoxy-C₁-C₄-alkyl, C₂-C₄-alkenyl, C₂-C₄-haloalkenyl, C₂-C₄-alkynyl, C₂-C₄-haloalkynyl, CN, CH₂CN, NR^{A}R^{B} or CH₂—O—C(═O)R′;
R¹, R² independently of each other are hydrogen, halogen, CN, OH, C₁-C₄-alkyl, C₁-C₄-haloalkyl, C₁-C₄-alkoxy, C₁-C₄-haloalkoxy, C₁-C₄-alkoxy-C₁-C₄-alkyl, C₁-C₄-haloalkoxy-C₁-C₄-alkyl, C₂-C₄-alkenyl, C₂-C₄-haloalkenyl, C₂-C₄-alkynyl, C₂-C₄-haloalkynyl, C₃-C₈-cycloalkyl, C₃-C₈-cycloalkyl-C₁-C₄-alkyl, C₃-C₈-cycloalkyloxy, NR^{A}R^{B}, C(═O)R′, C(═NOR″)R′″, —C(═NH)—O—R′″ or benzyl wherein the phenyl moiety of benzyl is unsubstituted or carries 1, 2, 3, 4, or 5 substituents selected from CN, halogen, C₁-C₄-alkyl, C₁-C₄-haloalkyl, C₁-C₄-alkoxy, C₁-C₄-haloalkoxy, (C₁-C₄-alkoxy)carbonyl and di(C₁-C₄-alkyl)aminocarbonyl; or
two radicals R¹ and R² that are bound to the same carbon atom form together with said carbon atom a saturated or partially unsaturated
3-, 4-, 5-, 6-, or 7-membered carbocycle or a saturated or partially unsaturated 3-, 4-, 5-, 6-, or 7-membered heterocycle, wherein the ring member atoms of the abovementioned heterocycle include beside carbon atoms 1, 2, 3 or 4 heteroatoms selected from N, O and S, and wherein the abovementioned cycle is unsubstituted or carries 1, 2, 3 or 4 substituents selected from halogen, CN, OH, SH, C₁-C₄-alkyl, C₁-C₄-alkoxy and C₁-C₄-alkylthio; and one or two CH₂ groups of the abovementioned cycles may respectively be replaced by one or two C(═O) or C(═S) groups;
R³, R⁴ independently of each other are hydrogen, CN, C₁-C₄-hydroxyalkyl, C₁-C₄-alkyl, C₁-C₄-haloalkyl, C₁-C₄-alkoxy, C₁-C₄-haloalkoxy, C₁-C₄-alkoxy-C₁-C₄-alkyl, C₁-C₄-haloalkoxy-C₁-C₄-alkyl, C₂-C₄-alkenyl, C₂-C₄-haloalkenyl, C₂-C₄-alkynyl, C₂-C₄-haloalkynyl, C₃-C₈-cycloalkyl, C₃-C₈-cycloalkyl-C₁-C₄-alkyl, C₃-C₈-cycloalkyloxy, NR^{A}R^{B}, C(═O)R′, C(═NOR″)R′″, —C(═NH)—O—R′″ or benzyl wherein the phenyl moiety of benzyl is unsubstituted or carries 1, 2, 3, 4, or 5 substituents selected from the group consisting of CN, halogen, C₁-C₄-alkyl, C₁-C₄-haloalkyl, C₁-C₄-alkoxy, C₁-C₄-haloalkoxy, (C₁-C₄-alkoxy)carbonyl and di(C₁-C₄-alkyl)aminocarbonyl; or
two radicals R³ and R⁴ that are bound to the same carbon atom form together with said carbon atom a saturated or partially unsaturated
3-, 4-, 5-, 6-, or 7-membered carbocycle or a saturated or partially unsaturated 3-, 4-, 5-, 6-, or 7-membered heterocycle, wherein the ring member atoms of the abovementioned heterocycle include beside carbon atoms 1, 2, 3 or 4 heteroatoms selected from the group of N, O and S, and wherein the abovementioned cycle is unsubstituted or carries 1, 2, 3 or 4 substituents selected from halogen, CN, OH, SH, C₁-C₄-alkyl, C₁-C₄-alkoxy or C₁-C₄-alkylthio; and one or two CH₂ groups of the abovementioned cycles may be respectively replaced by one or two C(═O) or C(═S) groups;
n is 0, 1, 2, 3 or 4;
R^{b} is independently selected from halogen, CN, NO₂, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl, C₂-C₄-haloalkynyl, NR^{A}R^{B}, C(═O)R′, C(═NOR″)R′ and —C(═NH)—O—R′″
Het is a 5- or 6-membered heteroaryl, wherein the ring member atoms of the heteroaryl include besides carbon atoms 1, 2, 3 or 4 heteroatoms selected from N, O and S, and wherein the heteroaryl is unsubstituted or carries 1, 2, 3 or 4 identical or different groups R^{c}:
R^{c} is halogen, CN, NO₂, NH₂, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C(═O)R′, C(═NOR″)R′″, C₃-C₈-cycloalkyl, C₃-C₈-cycloalkyl-C₁-C₄-alkyl, phenyl, phenoxy, phenoxy-C₁-C₄-alkyl or a 5- or 6-membered heteroaryl, wherein the ring member atoms of the heteroaryl include besides carbon atoms 1, 2, 3 or 4 heteroatoms selected from N, O and S, and wherein the aforementioned cyclic radicals are unsubstituted or carry 1, 2, 3 or 4 identical or different substituents R^{d}:
R^{d} is halogen, CN, C₁-C₄-alkyl, C₁-C₄-haloalkyl, C₁-C₄-alkoxy or C₁-C₄-haloalkoxy;
or two radicals R^{c} that are bound to adjacent ring member atoms of the Het group form together with said ring member atoms a fused
5-, 6- or 7-membered saturated, partially unsaturated or aromatic carbocycle or heterocycle, wherein the ring member atoms of the fused heterocycle include besides carbon atoms 1, 2, 3 or 4 heteroatoms selected from the group of N, O and S, and wherein the fused carbocycle or heterocycle is unsubstituted or carries 1, 2, 3 or 4 identical or different radicals groups R^{e}:
R^{e} is halogen, CN, C₁-C₄-alkyl, C₁-C₄-haloalkyl, C₁-C₄-alkoxy or C₁-C₄-haloalkoxy;
or an N-oxide or an agriculturally acceptable salt thereof, with the proviso that 6-chloro-5-methoxy-N-[1-methyl-4-[4-[[4-(trifluoromethyl)-2-pyridyl]oxy]phenyl]but-3-ynyl]pyrimidin-4-amine is excluded.

15: The compound of claim 14, wherein R^{a5} and R^{a6} independently of each other are halogen, CN, C₁-C₄-alkyl, C₁-C₄-haloalkyl, C₁-C₄-alkoxy, C₁-C₄-haloalkoxy, C₁-C₄-alkoxy-C₁-C₄-alkyl, C₁-C₄-alkoxy-C₁-C₄-alkoxy or (C₁-C₄-alkoxy)carbonyl, and it being possible that one of R^{a5} or R^{a6} can in addition be hydrogen.

16: The compound of claim 14, wherein the group —CR³R⁴— is —CH(CH₃)—, —CH(CH₂CH₃)—, —C(CH₃)₂—, —CHCN— or —CH(C(═O)—C₁-C₄-alkoxy).

17: The compound of claim 14, wherein the group —CR³R⁴— is —CH(CH₃)—.

18: The compound of claim 14, wherein Het is pyrimidin-2-yl, pyrimidin-3-yl, pyrimidin-4-yl, pyridin-2-yl, pyridin-3-yl, pyridin-4-yl, thiazol-2-yl, pyrazin-2-yl, pyridazin-3-yl, 1,3,5-triazin-2-yl, or 1,2,4-triazin-3-yl.

19: The compound of claim 14, wherein Het is pyrimidin-2-yl, pyrimidin-3-yl, pyrimidin-4-yl, pyridin-2-yl, pyridin-3-yl or pyridin-4-yl.

20: The compound of claim 14, wherein Het carries 1 or 2 radicals R^{c} which are selected from F, Cl, CN, CH₃, OCH₃, CF₃, CHF₂, OCF₃, OCHF₂ and COOCH₃.

21: A process for preparing the compound of claim 14, which comprises reacting a compound of formula II 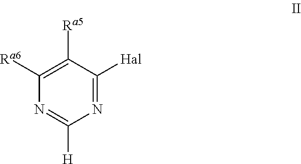
wherein Hal is fluorine, chlorine or bromine, with a compound of formula III 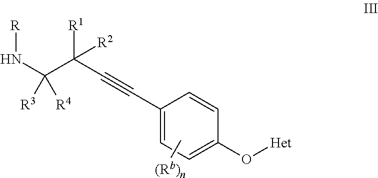
wherein in the presence of a base or a catalyst or a combination of a base and a catalyst.

22: An agrochemical composition which comprises an auxiliary and at least one compound of claim 14.

23: The agrochemical composition of claim 22 comprising at least one further active substance.

24: A method for combating phytopathogenic harmful fungi, which process comprises treating the fungi or the materials, plants, the soil or seeds to be protected against fungal attack, with an effective amount of at least one compound of formula I or an N-oxide or an agriculturally acceptable salt thereof, according to claim 14.

25: Seed treated with the compound of claim 14 in an amount of from 0.1 g to 10 kg active components per 100 kg of seed.

26: The method of claim 24, wherein R^{a5} and R^{a6} independently of each other are halogen, CN, C₁-C₄-alkyl, C₁-C₄-haloalkyl, C₁-C₄-alkoxy, C₁-C₄-haloalkoxy, C₁-C₄-alkoxy-C₁-C₄-alkyl, C₁-C₄-alkoxy-C₁-C₄-alkoxy or (C₁-C₄-alkoxy)carbonyl, and it being possible that one of R^{a5} or R^{a6} can in addition be hydrogen.

27: The method of claim 24, wherein the group —CR³R⁴— is —CH(CH₃)—, —CH(CH₂CH₃)—, —C(CH₃)₂—, —CHCN— or —CH(C(═O)—C₁-C₄-alkoxy).

28: The method of claim 24, wherein the group —CR³R⁴— is —CH(CH₃)—.

29: The method of claim 24, wherein Het is pyrimidin-2-yl, pyrimidin-3-yl, pyrimidin-4-yl, pyridin-2-yl, pyridin-3-yl, pyridin-4-yl, thiazol-2-yl, pyrazin-2-yl, pyridazin-3-yl, 1,3,5-triazin-2-yl, or 1,2,4-triazin-3-yl.

30: The method of claim 24, wherein Het is pyrimidin-2-yl, pyrimidin-3-yl, pyrimidin-4-yl, pyridin-2-yl, pyridin-3-yl or pyridin-4-yl.

31: The method of claim 24, wherein Het carries 1 or 2 radicals R^{c} which are selected from F, Cl, CN, CH₃, OCH₃, CF₃, CHF₂, OCF₃, OCHF₂ and COOCH₃.
